(19) United States
(12) Patent Application Publication
(10) Pub. No.: US 2026/0021070 A1
(43) Pub. Date: Jan. 22, 2026

(54) SUBCUTANEOUS FORMULATIONS COMPRISING PSILOCYBIN DERIVATIVES

(71) Applicant: Compass Pathfinder Limited, Altrincham (GB)

(72) Inventors: John Richard MORPHY, Altrincham (GB); Paul Stanley BURY, Harlow (GB); Niall Anthony WAGSTAFF, Harlow (GB); Paul Stuart HINCHLIFFE, Harlow (GB); Susan Elizabeth OSBOURN, Harlow (GB)

(21) Appl. No.: 19/273,639

(22) Filed: Jul. 18, 2025

Related U.S. Application Data
(60) Provisional application No. 63/673,523, filed on Jul. 19, 2024.

(51) Int. Cl. A61K 31/4045; A61K 9/00; A61K 47/02; A61K 47/26

(52) U.S. Cl. CPC A61K 31/4045; A61K 9/0019; A61K 47/02; A61K 47/26

(57) ABSTRACT

The present disclosure provides stable liquid formulations comprising Compound 1, or a pharmaceutically acceptable salt of Compound 1, and one or more pharmaceutically acceptable excipients. The liquid formulation may comprise a pH buffered aqueous solution, and optionally a pH modifier and/or a tonicity agent. Also provided herein are methods of delivering the formulation via parenteral administration (e.g., subcutaneous injection), and methods of using the formulation in the treatment of neuropsychiatric diseases (e.g., treatment resistant depression).

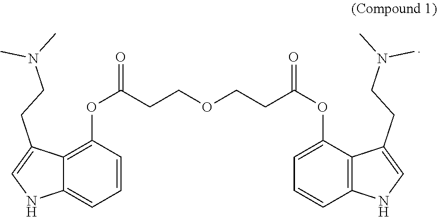

### CROSS-REFERENCE TO RELATED APPLICATION

This application claims the benefit of priority to U.S. Provisional Application No. 63/673,523, filed Jul. 19, 2024, which is hereby incorporated by reference in its entirety for all purposes.

### BACKGROUND

Over 50% of adults in the United States will be diagnosed with a psychiatric disorder at some point in their lifetime. Nearly 1 in 5 suffer from mental illness, and nearly 1 in 25 are afflicted with severe mental illness, such as major depression, schizophrenia, or bipolar disorder. Psilocybin shows promising activity in treating mental illness.

Provided the potential therapeutic applications, there is a need in the art for new pharmaceutical compositions that provide rapid onset of therapeutic activity and meet regulatory criteria for approved drug products.

### SUMMARY

Provided herein are pharmaceutical formulations that comprise psilocybin derivatives and pharmaceutically acceptable excipients including pH buffered aqueous solutions. The formulations can comprise additional pH modifiers and/or tonicity agents. In embodiments, the present disclosure relates to liquid formulations comprising an effective amount of Compound 1

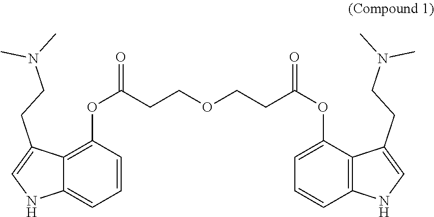

or an equivalent amount of a pharmaceutically acceptable salt thereof, and a pharmaceutically acceptable excipient.

In embodiments, the salt is a fumarate salt of Compound 1, a succinate salt of Compound 1, or a tartrate salt of Compound 1.

In embodiments, the salt is a monofumarate salt of Compound 1.

In embodiments, the salt is a sesquifumarate salt of Compound 1.

In embodiments, the salt is a succinate salt of Compound 1.

In embodiments, the salt is a L-tartrate salt of Compound 1.

In embodiments, the salt is a phosphate salt of Compound 1.

In embodiments, the liquid formulation comprises about 2 mg/ml to about 40 mg/mL of Compound 1, or an equivalent amount of a pharmaceutically acceptable salt thereof, relative to a total volume of the formulation. In embodiments, the formulation comprises about 30 mg/mL to about 40 mg/ml of Compound 1, or an equivalent amount of a pharmaceutically acceptable salt thereof, relative to a total volume of the formulation.

In embodiments, the liquid formulation comprises a pH buffered aqueous solution. In embodiments, the pH buffered aqueous solution is a phosphate buffer solution or a histidine buffer solution. In embodiments, the pH buffered aqueous solution is a phosphate buffer solution having a phosphate concentration of about 1 mM to about 100 mM and a pH ranging from about 5.5 to about 6.5. In embodiments, the pH buffered aqueous solution is a phosphate buffer solution having a phosphate concentration of about 10 mM and a pH of about 6.

In embodiments, the pharmaceutically acceptable excipient of the liquid formulation further comprises a pH modifier. In embodiments, the pH modifier is sodium hydroxide.

In embodiments, the liquid formulation has a pH ranging from about 5.8 to about 8.5. In embodiments, the formulation has a pH ranging from about 6 to about 7.5. In embodiments, wherein the formulation has a pH of 6.0±0.2, 6.5±0.2, or 7.4±0.2.

In embodiments, the liquid formulation is acidic. In embodiments, the formulation has a pH of 6.0±0.2.

In embodiments, the pharmaceutically acceptable excipient of the liquid formulation further comprises a tonicity agent. In embodiments, the tonicity agent is sodium chloride or mannitol. In embodiments, the tonicity agent is sodium chloride, and sodium chloride is present in the formulation in a concentration of about 0.1 wt % to about 5 wt %.

In embodiments, the liquid formulation has an osmolarity ranging from 200 mOsm/kg to 600 mOsm/kg. In embodiments, the formulation has an osmolarity ranging from about 280 mOsm/kg to about 400 mOsm/kg. In embodiments, the formulation has an osmolarity of about 300 mOsm/kg.

In embodiments, the liquid formulation is formulated for parenteral administration. In embodiments, the liquid formulation is formulated for subcutaneous administration.

In embodiments, the liquid formulation comprises:
about 30 mg/mL to about 40 mg/ml of Compound 1, or an equivalent amount of a pharmaceutically acceptable salt thereof, relative to a total volume of the formulation,
a phosphate buffer solution having a phosphate concentration of about 10 mM and a pH of about 6,
sodium hydroxide, and
about 0.1 wt % to about 5 wt % sodium chloride,
wherein the formulation is formulated for subcutaneous administration.

In embodiments, the liquid formulation comprises:
about 30 mg/mL to about 40 mg/ml of Compound 1 (as a free base) relative to a total volume of the formulation,
a phosphate buffer solution having a phosphate concentration of about 10 mM and a pH of about 6,
sodium hydroxide, and
about 0.1 wt % to about 5 wt % sodium chloride, wherein the formulation is formulated for subcutaneous administration.

In embodiments, the liquid formulation comprises:
about 30 mg/mL to about 40 mg/ml of sesquifumarate salt of Compound 1 relative to a total volume of the formulation, wherein the concentration is determined based on the weight of Compound 1 as a free base,
a phosphate buffer solution having a phosphate concentration of about 10 mM and a pH of about 6,
sodium hydroxide, and
about 0.1 wt % to about 5 wt % sodium chloride,
wherein the formulation is formulated for subcutaneous administration.

In embodiments, the liquid formulation has a shelf-life of at least 12 hours, 24 hours, or 48 hours at 25° C. In embodiments, the formulation has a shelf-life of at least 4 days, 7 days, 14 days, 30 days, 2 months, or 3 months at 5° C.

In embodiments, the present disclosure provides methods of delivering Compound 1 or a pharmaceutically acceptable salt thereof to a subject. The method comprises administering the liquid formulation disclosed herein to the subject parenterally.

In embodiments, the liquid formulation is administered to the subject subcutaneously. In embodiments, the liquid formulation is administered subcutaneously in a volume ranging from about 0.1 mL to about 2 mL.

In embodiments, the present disclosure provides methods of treating a neuropsychiatric disease. The method comprises parenterally administering the liquid formulation disclosed herein to a subject in need thereof.

In embodiments, the liquid formulation is administered to the subject subcutaneously. In embodiments, the liquid formulation is administered subcutaneously in a volume ranging from about 0.1 mL to about 2 mL.

In embodiments, the subject is a human.

In embodiments, the neuropsychiatric disorder is selected from anxiety disorder, attention deficit hyperactivity disorder (ADHD), depression (including treatment resistant depression), cluster headache, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, attention deficit syndrome, posttraumatic stress disorder (PTSD), premenstrual dysphoric disorder (PMDD), or premenstrual syndrome (PMS).

In embodiments, the present disclosure provides methods of making a liquid formulation for parenteral administration to a subject. The method comprises: combining an effective amount of a pharmaceutically acceptable salt of Compound 1;

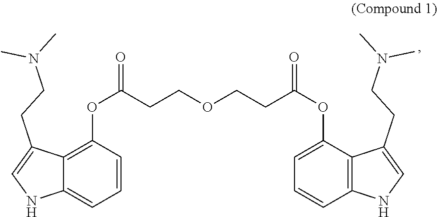

and
a pH buffered aqueous solution; and optionally a pharmaceutically acceptable excipient, thereby forming the liquid formulation.

In embodiments, the method comprises combining an effective amount of the pharmaceutically acceptable salt of Compound 1, the pH buffered aqueous solution, and the pharmaceutically acceptable excipient.

In embodiments, the salt of Compound 1 in the method of making is crystalline. In embodiments, the salt is a sesquifumarate salt of Compound 1. In embodiments, the sesquifumarate salt of Compound 1 is crystalline, and is characterized by an XRPD pattern having peaks at 8.7±0.2, 19.7±0.2, and 23.6±0.2 °2θ. In embodiments, the sesquifumarate salt of Compound 1 is further characterized by peaks in an XRPD pattern at 14.4±0.2, 21.8±0.2, and 22.2±0.2 °2θ. In embodiments, the sesquifumarate salt of Compound 1 is characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °20.

In embodiments, the pH buffered aqueous solution comprises a phosphate buffer solution or a histidine buffer solution. In embodiments, the pH buffered aqueous solution is a phosphate buffer solution having a phosphate concentration of about 1 mM to about 100 mM and a pH ranging from about 5.5 to about 6.5. In embodiments, the pH buffered aqueous solution is a phosphate buffer solution having a phosphate concentration of about 10 mM and a pH of about 6. In embodiments, the phosphate buffer solution is a phosphate-buffered saline (PBS).

In embodiments, the pharmaceutically acceptable excipient comprises a pH modifier. In embodiments, the pH modifier is sodium hydroxide. In embodiments, the pharmaceutically acceptable excipient further comprises a tonicity agent. In embodiments, the tonicity agent is sodium chloride or mannitol.

In embodiments, the formulation prepared by the method disclosed herein has a pH ranging from about 5.8 to about 8.5. In embodiments, the formulation has a pH ranging from about 6 to about 7.5. In embodiments, the formulation has a pH of 6.0±0.2, 6.5±0.2, or 7.4±0.2. In embodiments, the formulation is acidic. In embodiments, the formulation has a pH of 6.0±0.2.

In embodiments, the formulation prepared by the method disclosed herein has an osmolarity ranging from 200 mOsm/kg to 600 mOsm/kg. In embodiments, the formulation has an osmolarity ranging from about 280 mOsm/kg to about 400 mOsm/kg. In embodiments, the formulation has an osmolarity of about 300 mOsm/kg.

In embodiments, the formulation prepared by the method disclosed herein is formulated for subcutaneous administration.

In embodiments, the present disclosure provides a first container comprising about 1 mg to 100 mg of a pharmaceutically acceptable salt of Compound 1

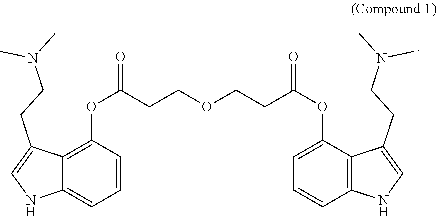

In embodiments, the salt of Compound 1 present in the first container is in crystalline form. In embodiments, the salt is a sesquifumarate salt of Compound 1. In embodiments, the sesquifumarate salt of Compound 1 is crystalline, and is characterized by an XRPD pattern having peaks at 8.7±0.2, 19.7±0.2, and 23.6±0.2 °2θ. In embodiments, the sesquifumarate salt of Compound 1 is further characterized by peaks in an XRPD pattern at 14.4±0.2, 21.8±0.2, and 22.2±0.2 °2θ. In embodiments, the sesquifumarate salt of Compound 1 is characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °2θ.

In embodiments, the present disclosure provides a kit comprising the first container disclosed herein in any embodiments and a second container comprising a pH buffered aqueous solution. In embodiments, the second container further comprises a pharmaceutically acceptable excipient.

In embodiments, the pH buffered aqueous solution present in the second container of the kit is a phosphate buffer solution having a phosphate concentration of about 1 mM to about 100 mM and a pH ranging from about 5.5 to about 6.5. In embodiments, the pH buffered aqueous solution is a phosphate-buffered saline (PBS).

In embodiments, the pharmaceutically acceptable excipient present in the second container of the kit comprises a pH modifier. In embodiments, the pH modifier is sodium hydroxide. In embodiments, the pharmaceutically acceptable excipient further comprises a tonicity agent. In embodiments, the tonicity agent is sodium chloride or mannitol.

### BRIEF DESCRIPTION OF THE DRAWINGS

FIG. 1 shows ¹H NMR characterization of the free base of Compound 1.

FIGS. 2A-2B show characterization of the bis-formate salt of Compound 1: XRPD (FIG. 2A) and DSC (FIG. 2B).

FIGS. 3A-3E show characterization of the fumarate (1.0 equivalents) of Compound 1: ¹HMR (FIG. 3A), XRPD (FIG. 3B), DSC (FIG. 3C), TGA (FIG. 3D), GVS (FIG. 3E), and XRPD pre and post GVS (FIG. 3F) for Form 1.

FIGS. 3G-30 show characterization of the various polymorphic forms of the fumarate (1.0 equivalents) of Compound 1: XRPD overlay of the forms (FIG. 3G), XRPD of Form 2 (FIG. 3H), XRPD of Form 3 (FIG. 3I), XRPD of Form 4 (FIG. 3J), XRPD of Form 5 (FIG. 3K), XRPD of Form 6 (FIG. 3L), XRPD of Form 7 (FIG. 3M), XRPD of Form 8 (FIG. 3N), and XRPD of Form 9 (FIG. 3O).

FIGS. 4A-41 show characterization of the sesquifumarate (1.5 equivalents) salt of Compound 1: XRPD (FIG. 4A), ¹H NMR (FIG. 4B), DSC (FIG. 4C), TGA (FIG. 4D), GVS (FIG. 4E), XRPD pre and post GVS (FIG. 4F), XRPD before and after freezer storage for 17 weeks (FIG. 4G), XRPD before and after freezer storage under stress conditions for 12 weeks (FIG. 4H), and XRPD overlay from polymorph assessment (FIG. 4I).

FIGS. 4J-4L show single crystal structures of the sesquifumarate salt (1.5 equivalents) salt of Compound 1: asymmetric crystal unit cell with atom labeling and thermal ellipsoids drawn at 50% probability (FIG. 4J) and two-unit cells with stick model representation (FIG. 4K-4L).

FIGS. 5A-5C show characterization of the succinate salt of Compound 1: XRPD (FIG. 5A), DSC (FIG. 5B), and TGA (FIG. 5C).

FIGS. 6A-6B show characterization of the L-tartaric acid salt of Compound 1: XRPD (FIG. 6A) and DSC (FIG. 6B).

FIG. 7 is a graph summarizing pH stability analysis of Compound 1 sesquifumarate salt in 50 mM phosphate buffer (see Example 7).

FIG. 8 is a graph summarizing stability of Compound 1 as a function of its concentration in 50 mM phosphate buffer (see Example 8).

FIG. 9 is a graph illustrating the process to adjust formulations comprising sesquifumarate salt of Compound 1 to pH 6.0 via addition of 1M NaOH as a function of % total volume (see Example 9).

FIG. 10 is a graph summarizing stability assessment of formulations comprising 40 mg/mL Compound 1 (free base) at various temperatures (5° C. and 25° C.) and pH conditions (pH 6.0, 6.5 and 7.4) (see Example 11).

FIG. 11 is a graph summarizing the impact of pH on the stability of the formulations disclosed herein (see Example 11).

FIG. 12 is a bar graph summarizing the impact of NaOH addition on chemical purity of Compound 1 (free base) (see Example 12).

FIG. 13 shows XRPD characterization of the succinate (1.5 equivalents) salt of Compound 1 (see Example 15).

### DETAILED DESCRIPTION

The present disclosure relates to stable formulations comprising Compound 1, or a pharmaceutically acceptable salt thereof, and a pharmaceutically acceptable excipient. In embodiments, the formulations are liquid formulations. The pharmaceutically acceptable excipient can include a pH buffered aqueous solution. The formulations can further include pH modifiers and/or tonicity agents. The formulations disclosed herein are suitable for parenteral delivery (e.g., subcutaneous injection) to subjects in need thereof. As described further below, the formulation of the present technology, which can contain a high concentration (e.g., 30-40 mg/mL) of Compound 1, exhibits enhanced stability and extended shelf-life and limits injection site reactions.

Additionally, the present disclosure provides methods of using the formulations for treating diseases or conditions including neuropsychiatric diseases, e.g., major depressive disorder.

### Definitions

While the following terms are believed to be well understood by one of ordinary skill in the art, the following definitions are set forth to facilitate explanation of the presently disclosed subject matter.

The term “about” when immediately preceding a numerical value means a range encompassing said numerical value plus or minus an acceptable amount of variation in the art (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Compound 1 is 3-(2-(dimethylamino)ethyl)-1H-inden-4-yl 3-(3-((1-(2-(dimethylamino)ethyl)-1H-inden-7-yl)oxy)-3-oxopropoxy) propanoate having the structure below:

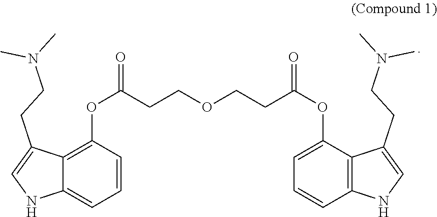

In this specification, unless stated otherwise, the term “pharmaceutically acceptable” is used to characterize a moiety (e.g., a salt, dosage form, or excipient) as being appropriate for therapeutic use. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound (e.g., Compound 1) functioning as a base, with an organic or inorganic acid to form a salt, for example, salts of fumaric acid, succinic acid, formic acid, tartaric acid, hydrochloric acid, sulfuric acid, phosphoric acid, methanesulfonic acid, camphorsulfonic acid, oxalic acid, maleic acid, citric acid, hydrobromic acid, benzoic acid, salicylic acid, mandelic acid, carbonic acid, etc. Those skilled in the art will further recognize that acid addition salts may be prepared by reaction of the compounds with the appropriate inorganic or organic acid via any of a number of known methods.

A “formulation” refers to a combination of substances including an active ingredient (e.g., Compound 1) and a component, such as a pharmaceutically acceptable excipient (e.g., buffers, pH modifiers, tonicity agents, binders, stabilizers, lubricants, oils, adjuvants, antioxidants, and other agents known in the art), to form a pharmaceutical composition.

The formulations of the present technology are suitable for administrations such as parenteral administration. The term parenteral as used here includes subcutaneous, intravenous, intramuscular, and intraarterial injections with a variety of infusion techniques. Intraarterial and intravenous injection as used herein includes administration through catheters.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human.

The terms “treating,” “treat,” and “treatment” as used herein with regard to a patient, refers to improving at least one symptom of the patient's disorder. Treating can be improving, or at least partially ameliorating a disorder or an associated symptom of a disorder.

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of Compound 1 is that amount that is required to reduce at least one symptom of depression in a patient. The actual amount that comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The term “therapeutic effect” as used herein refers to a desired or beneficial effect provided by the method and/or the composition. For example, the method for treating depression provides a therapeutic effect when the method reduces at least one symptom of depression in a patient.

The term “substantially similar” as used herein with regards to an analytical spectrum, such as an XRPD pattern, means that a spectrum resembles the reference spectrum in peak locations, allowing for variability appropriate in the art. For example, two spectra may be regarded as “substantially similar” when the two spectra share defining characteristics sufficient to differentiate them from a spectrum obtained for a different solid form. Relative peak intensities may vary depending on instrumentation and conditions, and such variations are taken into account when determining if two spectra are substantially similar. In embodiments, spectra or characterization data that are substantially similar to those of a reference crystalline form are understood by those of ordinary skill in the art to correspond to the same crystalline form as the particular reference. In analyzing whether spectra or characterization data are substantially similar, a person of ordinary skill in the art understands that particular characterization data points may vary to a reasonable extent while still describing a given solid form, due to, for example, experimental error and routine sample-to-sample analysis.

All XRPD peaks and patterns are given in °2θ using Cu Kα1 radiation at a wavelength of 1.5406 Å. The values of degree 2θ allow appropriate error margins. For example, the degree 2θ of about “17.5±0.2” denotes a range from about 17.3 to 17.7 degree 20. Depending on the sample preparation techniques, the calibration techniques applied to the instruments, human operational variation, and etc., those skilled in the art recognize that the appropriate error of margins for a XRPD can be ±0.2, which includes any value below ±0.2 such as ±0.1; ±0.05; or less.

TGA and DSC thermograms for a given form of the same Compound will vary within a margin of error. The values of a single peak, expressed in degree Celsius, allow appropriate error margins. Typically, the error margins are represented by “±”. For example, the single peak characteristic value of about “120±5” denotes a range from about 115 to 125. Depending on the sample preparation techniques, the calibration techniques applied to the instruments, human operational variations etc., those skilled in the art recognize that the appropriate error of margins for a single peak characteristic value can be ±5, which includes any value below ±5 such as ±4, ±3.5, ±3, ±2.5; ±2.0; ±1.5; ±1.0; ±0.5; or less.

The term “an equivalent amount”, as used herein, refers to an equal number of moles of two or more compounds. For example, an equivalent amount of about 30 mg of Compound 1 (i.e., about 0.056 mmol) can be about 39.9 mg of sesquifumarate salt of Compound 1 (i.e., about 0.056 mmol).

Unless otherwise specified, the concentrations of Compound 1 and salts thereof as described herein are determined based on the weight of Compound 1 as a free base. In other words, the concentrations of a salt of Compound 1 are determined based on the weight of an equivalent amount of Compound 1.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### Formulations

Aspects of the present disclosure relate to pharmaceutical formulations comprising a pharmaceutically acceptable excipient and Compound 1,

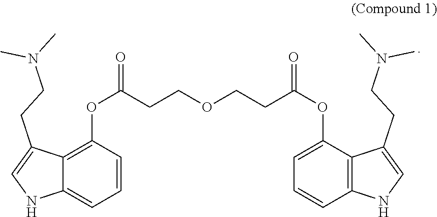

or a pharmaceutically acceptable salt form of Compound 1 disclosed herein in any of its embodiments. In embodiments, the formulation is a liquid formulation. In embodiments, the formulation is in the form of a solution. In embodiments, the formulation is in the form of a suspension.

### Compound 1 and Salts Thereof

In embodiments, the present disclosure relates to liquid formulations comprising an effective amount of Compound 1 and/or an equivalent amount of a pharmaceutically acceptable salt thereof.

In embodiments, the liquid formulation comprises Compound 1 as a free base.

In embodiments, the liquid formulation comprises a salt of Compound 1.

In embodiments, the salt of Compound 1 present in the formulation is a fumarate salt, a succinate salt, a tartrate salt, or a phosphate salt of Compound 1. In embodiments, the formulation of the present disclosure comprises a fumarate salt of Compound 1, a succinate salt of Compound 1, or a L-tartrate salt of Compound 1. In embodiments, the fumarate salt is a monofumarate salt, sesquifumarate salt, or a bis-formate salt of Compound 1. In embodiments, the formulation of the present disclosure comprises a phosphate salt of Compound 1. In embodiments, crystalline forms of salts of Compound 1 may be used to prepare the liquid formulations described herein.

In embodiments, the liquid formulation of the present disclosure comprises a monofumarate salt of Compound 1. In embodiments, the monofumarate salt of Compound 1 has the following formula:

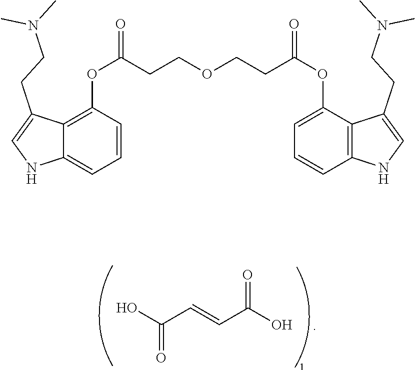

In embodiments, monofumarate salt of Compound 1 is crystalline.

In embodiments, the monofumarate salt of Compound 1 is crystalline Form

1, Form 2, Form 3, Form 4, Form 5, Form 6, Form 7, Form 8, Form 9, or mixtures thereof.

### Form 1:

In embodiments, the monofumarate salt of Compound 1, is characterized by an XRPD pattern having peaks at 12.8±0.2, 18.7±0.2, and 23.2±0.2 °2θ. In embodiments, the variance at any of these peaks is +0.1 °20.

In embodiments, the monofumarate salt of Compound 1 is characterized by an XRPD having peaks at 12.8±0.2, 18.7±0.2, and 23.2±0.2 °2θ and at least one (e.g., 1, 2, 3, 4, or 5) peak in an XRPD pattern selected from 7.2±0.2, 10.4±0.2, 17.9±0.2, 19.3±0.2, and 21.4±0.2 °2θ. In embodiments, the variance at any of these peaks is ±0.1 °2θ.

In embodiments, the monofumarate salt of Compound 1 is characterized by an XRPD having peaks at 7.2±0.2, 10.4±0.2, 12.8±0.2, 18.7±0.2, 17.9±0.2, 19.3±0.2, 21.4±0.2, and 23.2±0.2, 20. In embodiments, the variance at any of these peaks is ±0.1 °2θ.

In embodiments, the monofumarate salt of Compound 1 is characterized by an XRPD pattern having one or more peaks at 5.3±0.2, 7.2±0.2, 7.7±0.2, 8.9±0.2, 10.4±0.2, 11.3±0.2, 12.8±0.2, 13.7±0.2, 14.4±0.2, 14.7±0.2, 15.4±0.2, 16.5±0.2, 16.9±0.2, 17.3±0.2, 17.9±0.2, 18.7±0.2, 19.3±0.2, 19.8±0.2, 20.8±0.2, 21.0±0.2, 21.4±0.2, 21.8±0.2, 22.8±0.2, 23.2±0.2, 24.5±0.2, 24.8±0.2, 25.7±0.2, 26.4±0.2, 27.4±0.2, 28.6±0.2, 29.0±0.2, 29.5±0.2, 30.2±0.2, 30.6±0.2, 31.3±0.2, 32.3±0.2, and 32.7±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

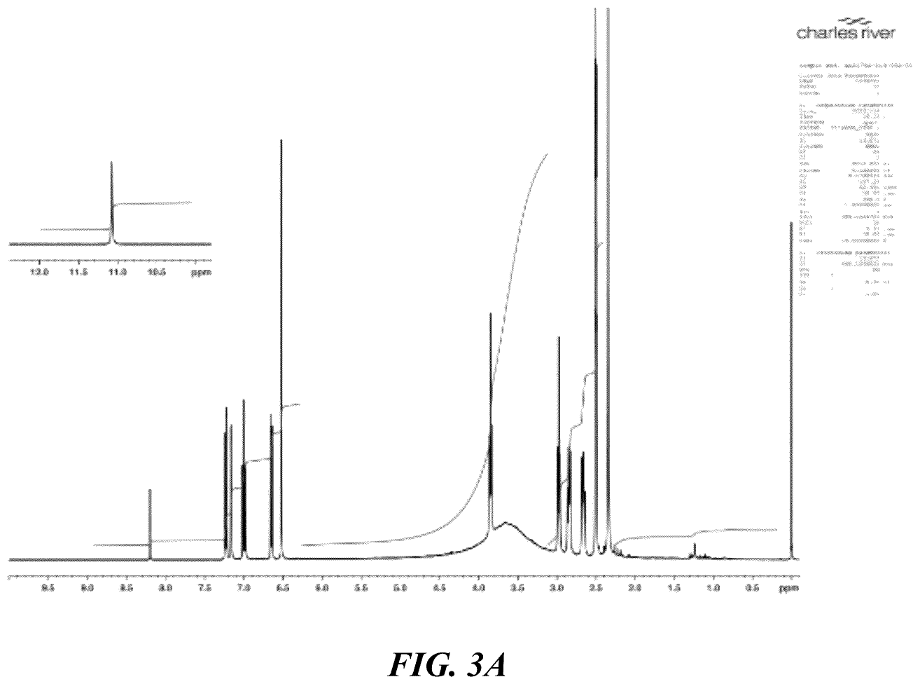

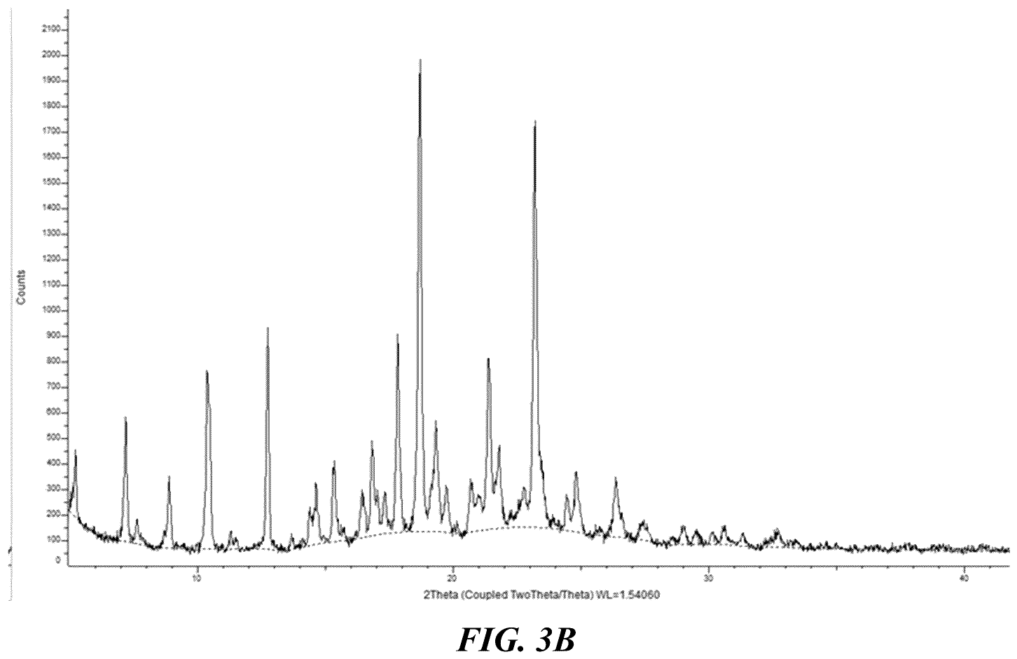

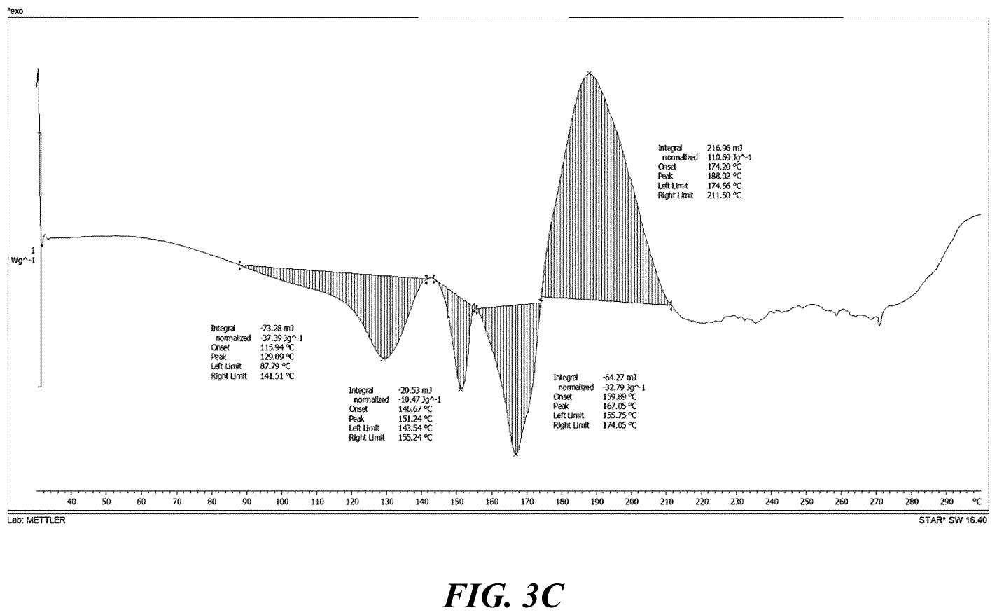

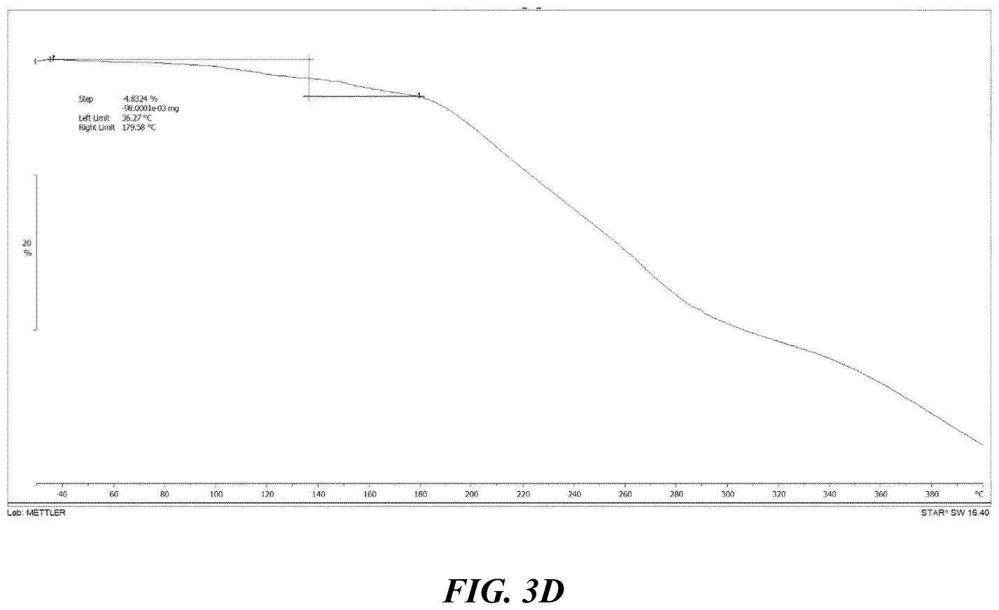

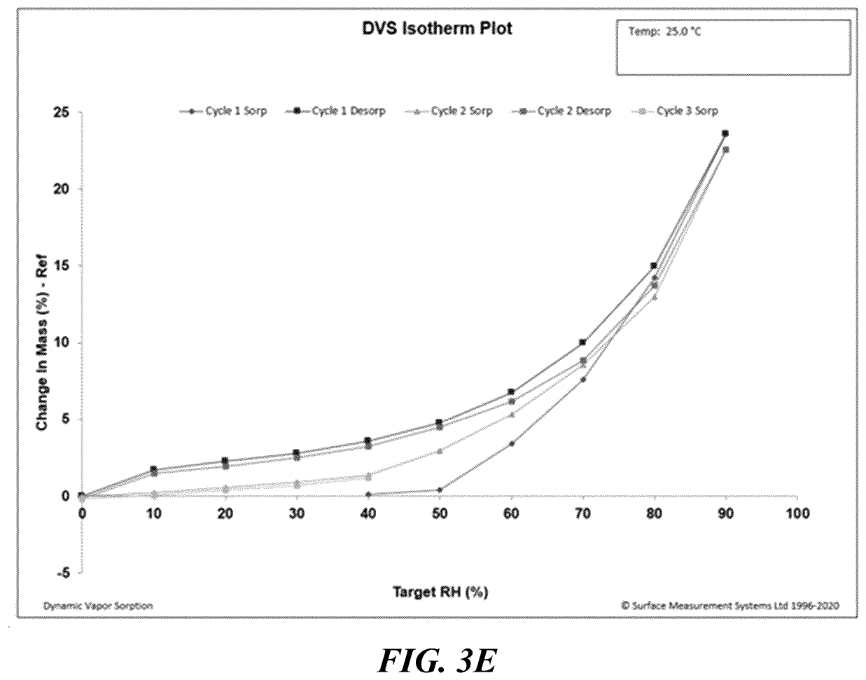

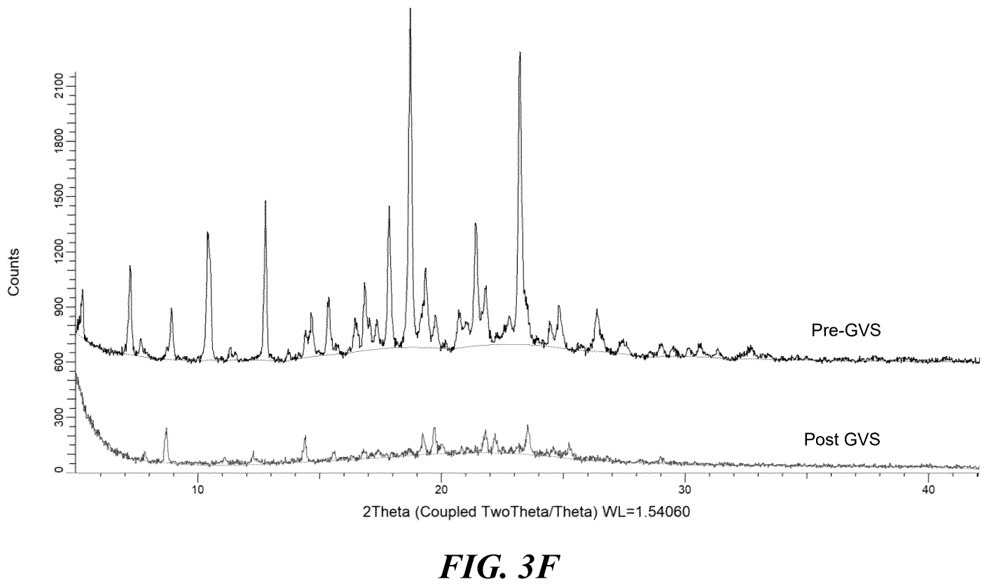

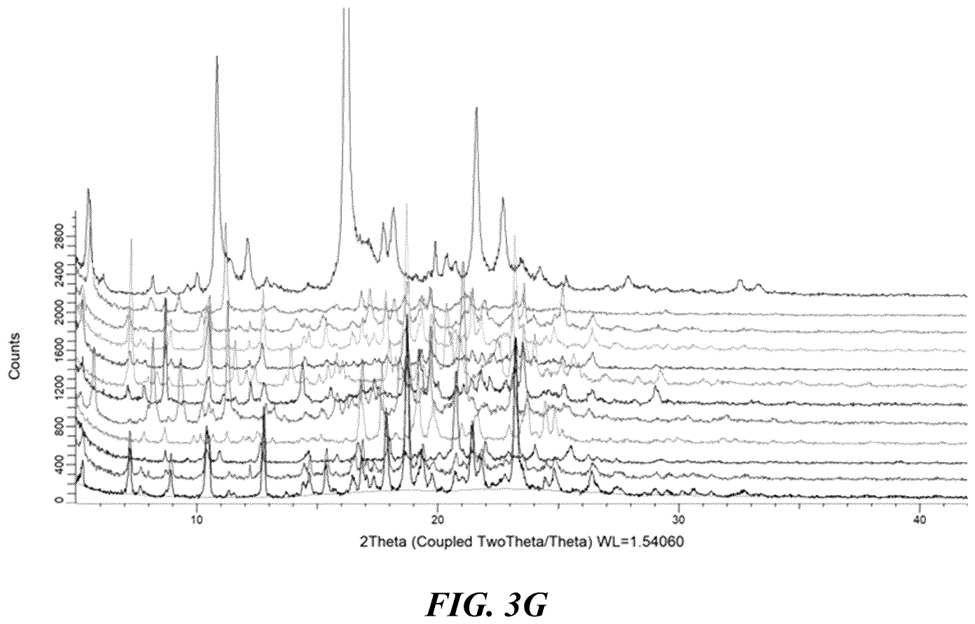

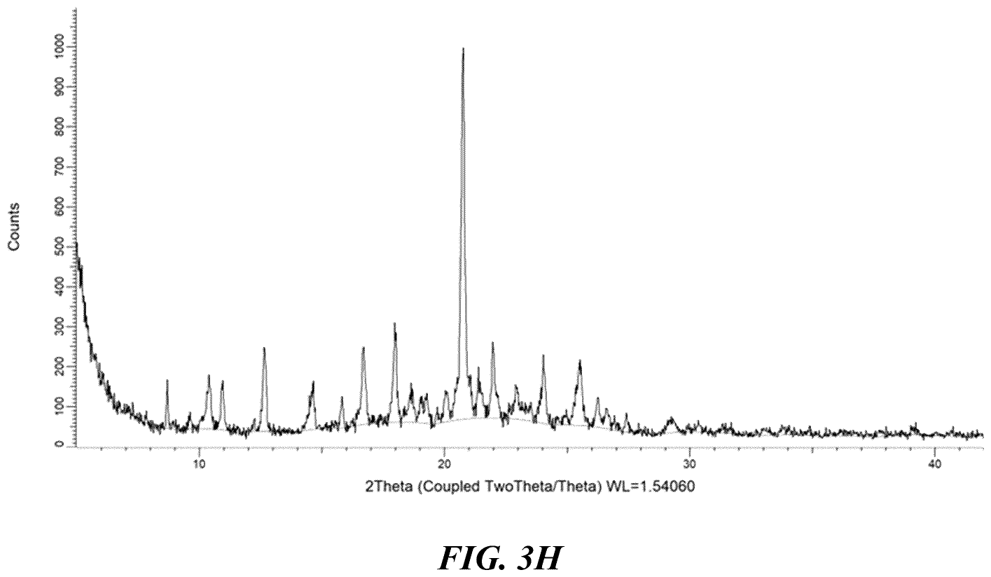

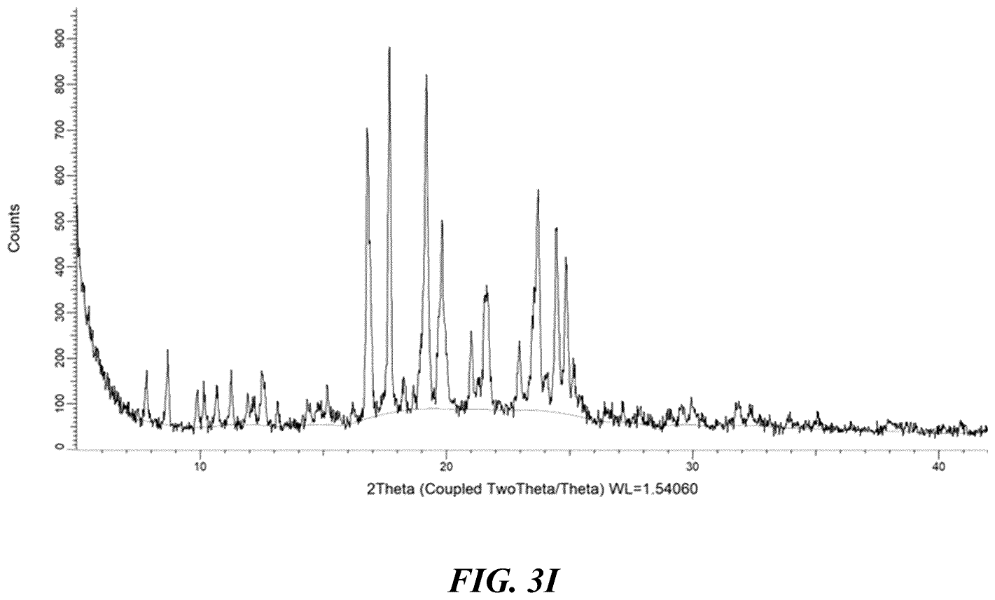

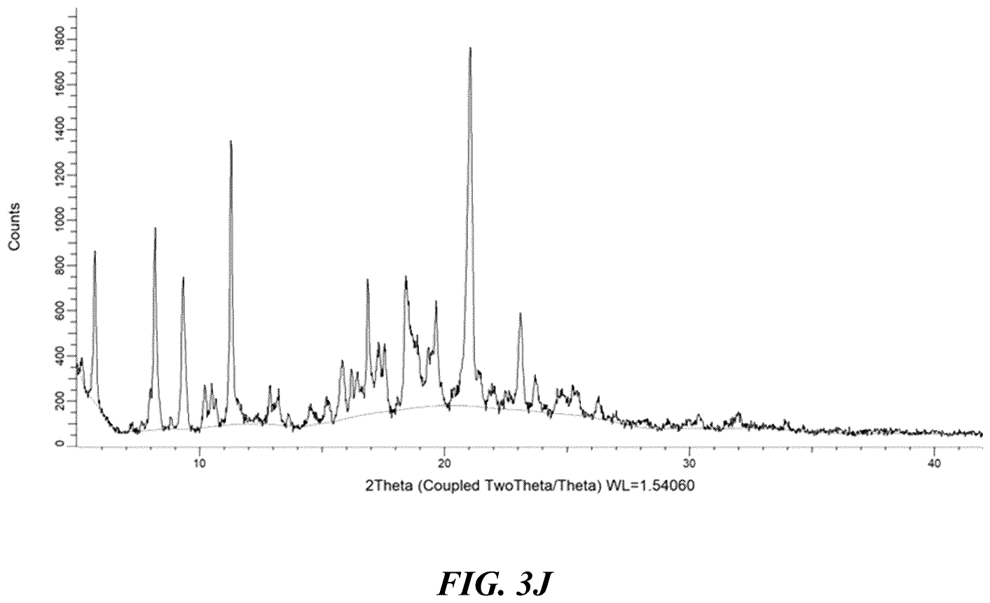

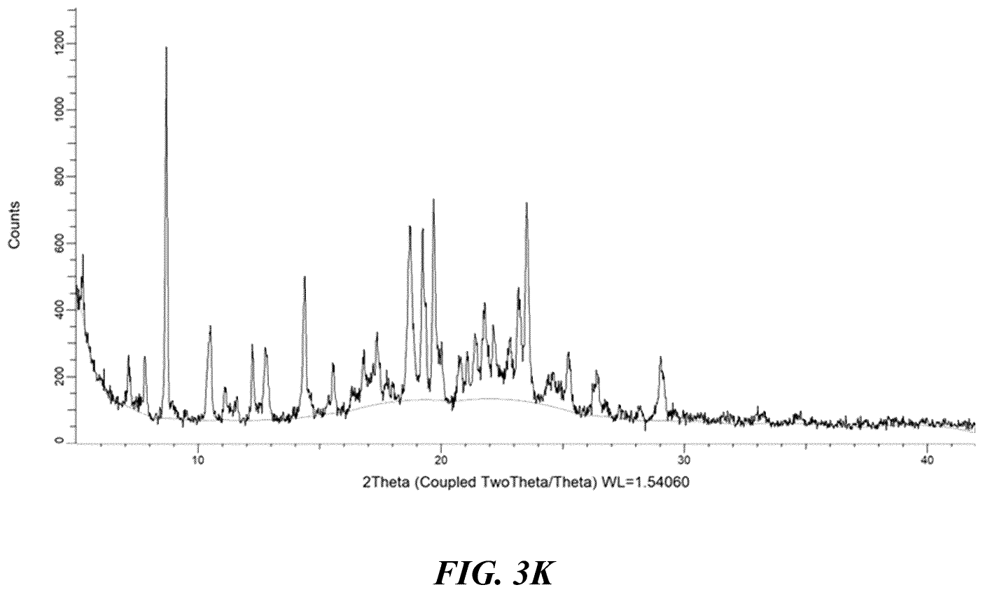

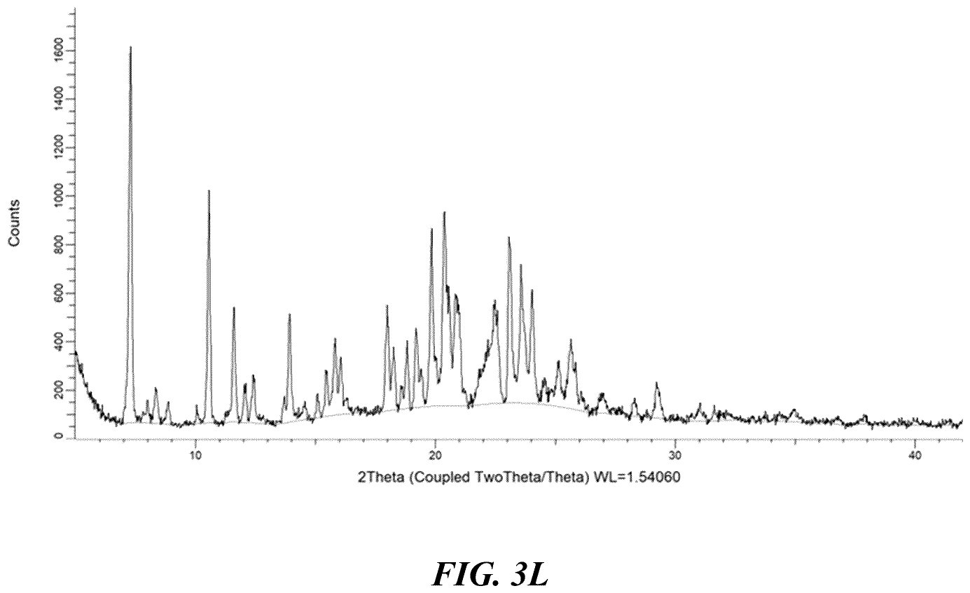

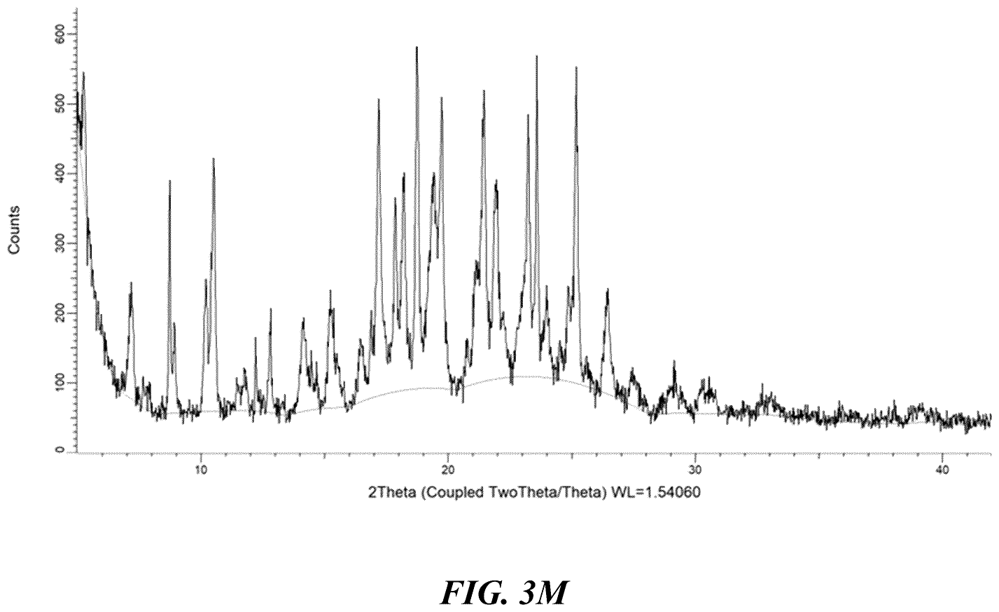

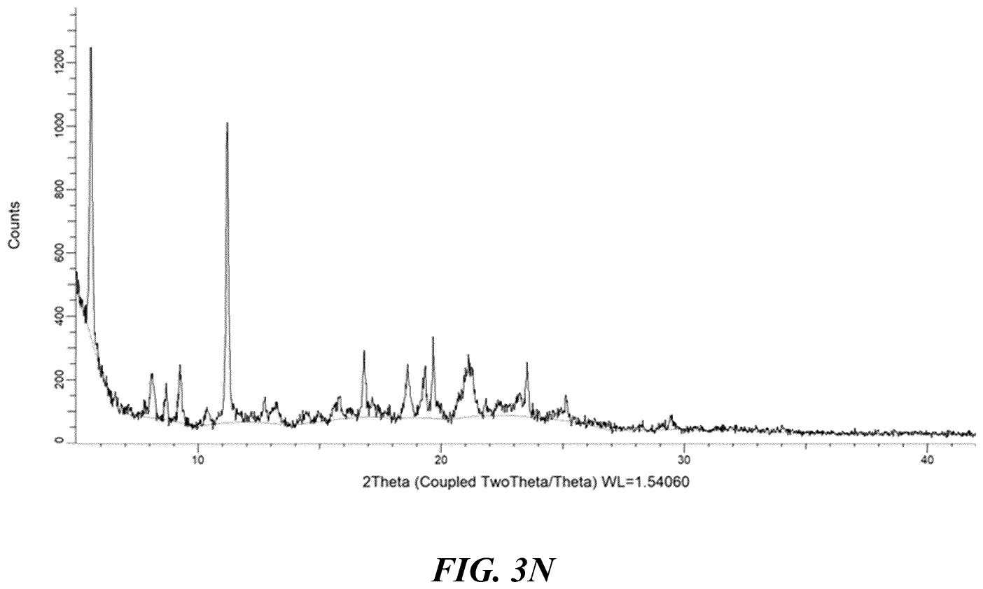

In embodiments, the monofumarate salt of Compound 1 is characterized by an XRPD pattern substantially similar to FIG. 3B.

In embodiments, monofumarate salt of Compound 1 is characterized by an XRPD pattern comprising peaks shown in Table A.

TABLE A
XRPD Peak Positions for the Monofumarate Salt of Compound 1

| Position °2θ | Relative Intensity |
|---|---|
| 5.3 | 11.34% |
| 7.2 | 24.13% |
| 7.7 | 4.88% |
| 8.9 | 15.20% |
| 10.4 | 33.56% |
| 11.3 | 3.44% |
| 12.8 | 45.24% |
| 13.7 | 2.41% |
| 14.4 | 7.27% |
| 14.7 | 10.87% |
| 15.4 | 15.15% |
| 16.5 | 8.66% |
| 16.9 | 19.21% |
| 17.3 | 8.17% |
| 17.9 | 40.66% |
| 18.7 | 100.00% |
| 19.3 | 24.48% |
| 19.8 | 9.10% |
| 20.8 | 9.06% |
| 21.0 | 6.36% |
| 21.4 | 35.02% |
| 21.8 | 15.97% |
| 22.8 | 8.61% |
| 23.2 | 84.94% |
| 24.5 | 6.25% |
| 24.8 | 13.77% |
| 25.7 | 2.20% |
| 26.4 | 12.23% |
| 27.4 | 3.71% |
| 28.6 | 1.57% |
| 29.0 | 3.41% |
| 29.5 | 2.22% |
| 30.2 | 1.93% |
| 30.6 | 2.15% |
| 31.3 | 3.14% |
| 32.3 | 1.15% |
| 32.7 | 4.60% |

In embodiments, monofumarate salt of Compound 1 is characterized by one, two, three, four, five, six, seven, eight, nine, ten, eleven, or more, XRPD peaks listed in Table A.

In embodiments, the monofumarate salt of Compound 1 is characterized by a DSC thermogram comprising an endothermic event between 88±5° C. to 142±5° C., 144±5° C. to 155±5° C., and 156±5° C. to 174±5° C.

In embodiments, the monofumarate salt of Compound 1 is characterized by a DSC comprising an exothermic event between 175±5° C. to 212±5° C.

In embodiments, the monofumarate salt of Compound 1 is characterized by a DSC thermogram substantially similar to FIG. 3C.

In embodiments the monofumarate salt of Compound 1 is characterized by about a 4.8% weight loss from 36±5° C. to 180±5° C. as determined by TGA.

In embodiments, the monofumarate salt of Compound 1 is characterized by a thermogravimetric thermogram substantially similar to FIG. 3D.

In embodiments, the monofumarate salt of Compound 1 of Form I has a chemical purity of greater than about 80%, about 85%, about 90%, about 95%, about 96%, about 97%, about 98%, about 99%, 99.5%, or about 100% by weight as determined by HPLC.

### Form 2:

In embodiments, the monofumarate salt of Compound 1, is characterized by an XRPD pattern having peaks at 12.7±0.2, 18.0±0.2, and 20.7±0.2 °2θ. In embodiments, the variance at any of these peaks is ±0.1 °2θ.

In embodiments, the monofumarate salt of Compound 1 is characterized by an XRPD having peaks at 12.7±0.2, 18.0±0.2, and 20.7±0.2 °2θ and at least one (e.g., 1, 2, 3, 4, or 5) peak in an XRPD pattern selected from 10.4±0.2, 16.7±0.2, 22.0±0.2, 24.0±0.2, and 25.5±0.2 °2θ. In embodiments, the variance at any of these peaks is ±0.1 °2θ.

In embodiments, the monofumarate salt of Compound 1 is characterized by an XRPD having peaks at 10.4±0.2, 12.7±0.2, 16.7±0.2, 18.0±0.2, 20.7±0.2, 22.0±0.2, 24.0±0.2, and 25.5±0.2 °2θ. In embodiments, the variance at any of these peaks is ±0.1 °2θ.

In embodiments, the monofumarate salt of Compound 1 is characterized by an XRPD pattern having one or more peaks at 8.7±0.2, 9.6±0.2, 10.4±0.2, 10.9±0.2, 12.7±0.2, 14.6±0.2, 15.8±0.2, 16.7±0.2, 18.0±0.2, 18.6±0.2, 19.1±0.2, 19.3±0.2, 19.7±0.2, 20.1±0.2, 20.7±0.2, 21.4±0.2, 22.0±0.2, 22.9±0.2, 23.4±0.2, 24.0±0.2, 25.5±0.2, 26.3±0.2, 26.6±0.2, 27.4±0.2, 29.3±0.2, 30.3±0.2, 33.8±0.2, 34.9±0.2, and 37.7±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3H.

In embodiments, monofumarate salt of Compound 1 is characterized by an XRPD pattern comprising peaks shown in Table B.

TABLE B
XRPD Peak Positions for the Monofumarate
Salt of Compound 1 (“Form 2”)

| Peak Position °2θ | Relative Intensity |
|---|---|
| 8.7 | 12.85% |
| 9.6 | 4.13% |
| 10.4 | 13.58% |
| 10.9 | 9.76% |
| 12.7 | 20.97% |
| 14.6 | 11.27% |
| 15.8 | 8.87% |
| 16.7 | 19.62% |
| 18.0 | 21.19% |
| 18.6 | 11.97% |
| 19.1 | 4.43% |
| 19.3 | 7.50% |
| 19.7 | 3.27% |
| 20.1 | 6.86% |
| 20.7 | 100.00% |
| 21.4 | 4.40% |
| 22.0 | 20.49% |
| 22.9 | 9.62% |
| 23.4 | 3.73% |
| 24.0 | 20.02% |
| 25.5 | 15.93% |
| 26.3 | 8.18% |
| 26.6 | 5.83% |
| 27.4 | 6.40% |
| 29.3 | 2.53% |
| 30.3 | 3.87% |
| 33.8 | 2.28% |
| 34.9 | 3.20% |
| 37.7 | 2.30% |

In embodiments, monofumarate salt of Compound 1 is characterized by one, two, three, four, five, six, seven, eight, nine, ten, eleven, or more, XRPD peaks listed in Table B.

In embodiments, the monofumarate salt of Compound 1 of Form 2 has a chemical purity of greater than about 80%, about 85%, about 90%, about 95%, about 96%, about 97%, about 98%, about 99%, 99.5%, or about 100% by weight as determined by HPLC.

### Form 3:

In embodiments, the monofumarate salt of Compound 1, is characterized by an XRPD pattern having peaks at 16.8±0.2, 17.7±0.2, and 19.2±0.2 °2θ. In embodiments, the variance at any of these peaks is ±0.1 °2θ.

In embodiments, the monofumarate salt of Compound 1 is characterized by an XRPD having peaks at 16.8±0.2, 17.7±0.2, and 19.2±0.2 °2θ and at least one (e.g., 1, 2, 3, 4, or 5) peak in an XRPD pattern selected from 19.8±0.2, 21.6±0.2, 23.7±0.2, 24.5±0.2, and 24.9±0.2 °2θ. In embodiments, the variance at any of these peaks is ±0.1 °20.

In embodiments, the monofumarate salt of Compound 1 is characterized by an XRPD having peaks at 16.8±0.2, 17.7±0.2, 19.2±0.2, 19.8±0.2, 21.6±0.2, 23.7±0.2, 24.5±0.2, and 24.9±0.2 °2θ. In embodiments, the variance at any of these peaks is ±0.1 °2θ.

In embodiments, the monofumarate salt of Compound 1 is characterized by an XRPD pattern having one or more peaks at 7.8±0.2, 8.7±0.2, 9.9±0.2, 10.2±0.2, 10.7±0.2, 11.3±0.2, 11.9±0.2, 12.1±0.2, 12.5±0.2, 13.1±0.2, 14.4±0.2, 15.2±0.2, 16.2±0.2, 16.8±0.2, 17.7±0.2, 18.3±0.2, 19.2±0.2, 19.8±0.2, 21.0±0.2, 21.6±0.2, 23.0±0.2, 23.7±0.2, 24.0±0.2, 24.5±0.2, 24.9±0.2, 25.2±0.2, 26.5±0.2, 27.2±0.2, 27.9±0.2, 29.1±0.2, 29.5±0.2, 29.9±0.2, 31.9±0.2, 32.3±0.2, 34.0±0.2, and 35.1±0.2 °20. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3I.

In embodiments, monofumarate salt of Compound 1 is characterized by an XRPD pattern comprising peaks shown in Table C.

TABLE C
XRPD Peak Positions for the Monofumarate
Salt of Compound 1 (“Form 3”)

| Peak Position °2θ | Relative Intensity |
|---|---|
| 7.8 | 12.93% |
| 8.7 | 20.99% |
| 9.9 | 8.79% |
| 10.2 | 11.32% |
| 10.7 | 7.11% |
| 11.3 | 14.18% |
| 11.9 | 7.98% |
| 12.1 | 7.99% |
| 12.5 | 12.44% |
| 13.1 | 5.09% |
| 14.4 | 5.07% |
| 15.2 | 11.39% |
| 16.2 | 5.61% |
| 16.8 | 71.96% |
| 17.7 | 100.00% |
| 18.3 | 9.28% |
| 19.2 | 88.78% |
| 19.8 | 51.48% |
| 21.0 | 20.80% |
| 21.6 | 33.44% |
| 23.0 | 18.27% |
| 23.7 | 58.01% |
| 24.0 | 8.01% |
| 24.5 | 53.85% |
| 24.9 | 38.47% |
| 25.2 | 10.61% |
| 26.5 | 2.46% |
| 27.2 | 5.41% |
| 27.9 | 4.70% |
| 29.1 | 4.37% |
| 29.5 | 4.63% |
| 29.9 | 7.91% |
| 31.9 | 6.67% |
| 32.3 | 3.38% |
| 34.0 | 3.86% |
| 35.1 | 5.43% |

In embodiments, monofumarate salt of Compound 1 is characterized by one, two, three, four, five, six, seven, eight, nine, ten, eleven, or more, XRPD peaks listed in Table C.

In embodiments, the monofumarate salt of Compound 1 of Form 3 has a chemical purity of greater than about 80%, about 85%, about 90%, about 95%, about 96%, about 97%, about 98%, about 99%, 99.5%, or about 100% by weight as determined by HPLC.

### Form 4:

In embodiments, the monofumarate salt of Compound 1, is characterized by an XRPD pattern having peaks at 8.2±0.2, 11.3±0.2, and 21.0±0.2 °2θ. In embodiments, the variance at any of these peaks is ±0.1 °2θ.

In embodiments, the monofumarate salt of Compound 1 is characterized by an XRPD having peaks at 8.2±0.2, 11.3±0.2, and 21.0±0.2 °2θ and at least one (e.g., 1, 2, 3, 4, or 5) peak in an XRPD pattern selected from 5.7±0.2, 9.3±0.2, 16.9±0.2, 18.5±0.2, and 19.7±0.2 °2θ. In embodiments, the variance at any of these peaks is ±0.1 °2θ.

In embodiments, the monofumarate salt of Compound 1 is characterized by an XRPD having peaks at 5.7±0.2, 8.2±0.2, 9.3±0.2, 11.3±0.2, 16.9±0.2, 18.5±0.2, 19.7±0.2, and 21.0±0.2 °2θ. In embodiments, the variance at any of these peaks is ±0.1 °2θ.

In embodiments, the monofumarate salt of Compound 1 is characterized by an XRPD pattern having one or more peaks at 5.7±0.2, 7.2±0.2, 7.6±0.2, 8.2±0.2, 8.8±0.2, 9.3±0.2, 10.2±0.2, 10.5±0.2, 11.3±0.2, 12.3±0.2, 12.9±0.2, 13.2±0.2, 13.6±0.2, 14.5±0.2, 15.2±0.2, 15.8±0.2, 16.2±0.2, 16.4±0.2, 16.9±0.2, 17.3±0.2, 17.6±0.2, 18.5±0.2, 18.9±0.2, 19.4±0.2, 19.7±0.2, 20.4±0.2, 21.0±0.2, 21.4±0.2, 21.9±0.2, 22.6±0.2, 23.1±0.2, 23.7±0.2, 24.8±0.2, 25.3±0.2, 25.4±0.2, 26.3±0.2, 27.0±0.2, 28.2±0.2, 29.1±0.2, 30.4±0.2, 32.0±0.2, and 33.9±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3J.

In embodiments, monofumarate salt of Compound 1 is characterized by an XRPD pattern comprising peaks shown in Table D.

TABLE D
XRPD Peak Positions for the Monofumarate
Salt of Compound 1 (“Form 4”)

| Peak Position °2θ | Relative Intensity |
|---|---|
| 5.7 | 37.45% |
| 7.2 | 2.05% |
| 7.6 | 1.83% |
| 8.2 | 53.95% |
| 8.8 | 3.01% |
| 9.3 | 40.38% |
| 10.2 | 11.58% |
| 10.5 | 12.79% |
| 11.3 | 79.17% |
| 12.3 | 1.75% |
| 12.9 | 10.64% |
| 13.2 | 7.05% |
| 13.6 | 2.79% |
| 14.5 | 3.41% |
| 15.2 | 3.97% |
| 15.8 | 16.37% |
| 16.2 | 12.43% |
| 16.4 | 12.71% |
| 16.9 | 35.13% |
| 17.3 | 18.45% |
| 17.6 | 17.29% |
| 18.5 | 32.84% |
| 18.9 | 20.66% |
| 19.4 | 16.30% |
| 19.7 | 30.11% |
| 20.4 | 5.76% |
| 21.0 | 100.00% |
| 21.4 | 9.44% |
| 21.9 | 5.79% |
| 22.6 | 2.79% |
| 23.1 | 26.82% |
| 23.7 | 9.43% |
| 24.8 | 4.19% |
| 25.3 | 5.42% |
| 25.4 | 5.79% |
| 26.3 | 4.18% |
| 27.0 | 2.23% |
| 28.2 | 1.76% |
| 29.1 | 1.74% |
| 30.4 | 4.86% |
| 32.0 | 3.82% |
| 33.9 | 2.36% |

In embodiments, monofumarate salt of Compound 1 is characterized by one, two, three, four, five, six, seven, eight, nine, ten, eleven, or more, XRPD peaks listed in Table D.

In embodiments, the monofumarate salt of Compound 1 of Form 4 has a chemical purity of greater than about 80%, about 85%, about 90%, about 95%, about 96%, about 97%, about 98%, about 99%, 99.5%, or about 100% by weight as determined by HPLC.

### Form 5:

In embodiments, the monofumarate salt of Compound 1, is characterized by an XRPD pattern having peaks at 8.7±0.2, 19.7±0.2, and 23.5±0.2 °2θ. In embodiments, the variance at any of these peaks is ±0.1 °2θ.

In embodiments, the monofumarate salt of Compound 1 is characterized by an XRPD having peaks at 8.7±0.2, 19.7±0.2, and 23.5±0.2 °2θ and at least one (e.g., 1, 2, 3, 4, or 5) peak in an XRPD pattern selected from 14.4±0.2, 18.7±0.2, 19.2±0.2, 21.8±0.2, and 23.2±0.2 °2θ. In embodiments, the variance at any of these peaks is ±0.1 °2θ.

In embodiments, the monofumarate salt of Compound 1 is characterized by an XRPD having peaks at 8.7±0.2, 14.4±0.2, 18.7±0.2, 19.2±0.2, 19.7±0.2, 21.8±0.2, 23.2±0.2, and 23.5±0.2 °2θ. In embodiments, the variance at any of these peaks is ±0.1 °2θ.

In embodiments, the monofumarate salt of Compound 1 is characterized by an XRPD pattern having one or more peaks at 7.1±0.2, 7.8±0.2, 8.7±0.2, 9.5±0.2, 10.5±0.2, 11.1±0.2, 11.6±0.2, 12.2±0.2, 12.8±0.2, 14.4±0.2, 15.6±0.2, 16.4±0.2, 16.8±0.2, 17.4±0.2, 17.7±0.2, 18.7±0.2, 19.2±0.2, 19.7±0.2, 20.0±0.2, 20.8±0.2, 21.1±0.2, 21.4±0.2, 21.8±0.2, 22.2±0.2, 22.8±0.2, 23.2±0.2, 23.5±0.2, 24.6±0.2, 25.2±0.2, 26.4±0.2, 26.7±0.2, 28.2±0.2, 29.0±0.2, 33.3±0.2, and 34.8±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3K.

In embodiments, monofumarate salt of Compound 1 is characterized by an XRPD pattern comprising peaks shown in Table E.

TABLE E
XRPD Peak Positions for the Monofumarate
Salt of Compound 1 (“Form 5”)

| Peak Position °2θ | Relative Intensity |
|---|---|
| 7.1 | 12.12% |
| 7.8 | 14.56% |
| 8.7 | 100.00% |
| 9.5 | 2.42% |
| 10.5 | 21.83% |
| 11.1 | 7.58% |
| 11.6 | 6.80% |
| 12.2 | 19.22% |
| 12.8 | 17.71% |
| 14.4 | 38.88% |
| 15.6 | 10.92% |
| 16.4 | 4.91% |
| 16.8 | 14.28% |
| 17.4 | 18.84% |
| 17.7 | 8.77% |
| 18.7 | 45.47% |
| 19.2 | 48.87% |
| 19.7 | 56.75% |
| 20.0 | 18.65% |
| 20.8 | 11.74% |
| 21.1 | 13.79% |
| 21.4 | 18.35% |
| 21.8 | 26.15% |
| 22.2 | 21.53% |
| 22.8 | 16.61% |
| 23.2 | 33.61% |
| 23.5 | 54.79% |
| 24.6 | 8.57% |
| 25.2 | 16.09% |
| 26.4 | 11.70% |
| 26.7 | 4.25% |
| 28.2 | 3.69% |
| 29.0 | 20.56% |
| 33.3 | 3.27% |
| 34.8 | 4.14% |

In embodiments, monofumarate salt of Compound 1 is characterized by one, two, three, four, five, six, seven, eight, nine, ten, eleven, or more, XRPD peaks listed in Table E.

In embodiments, the monofumarate salt of Compound 1 of Form 5 has a chemical purity of greater than about 80%, about 85%, about 90%, about 95%, about 96%, about 97%, about 98%, about 99%, 99.5%, or about 100% by weight as determined by HPLC.

### Form 6:

In embodiments, the monofumarate salt of Compound 1, is characterized by an XRPD pattern having peaks at 7.3±0.2, 10.6±0.2, and 19.8±0.2 °2θ. In embodiments, the variance at any of these peaks is ±0.1 °2θ.

In embodiments, the monofumarate salt of Compound 1 is characterized by an XRPD having peaks at 7.3±0.2, 10.6±0.2, and 19.8±0.2 °2θ and at least one (e.g., 1, 2, 3, 4, or 5) peak in an XRPD pattern selected from 13.9±0.2, 18.0±0.2, 20.4±0.2, 23.1±0.2, and 23.6±0.2 °2θ. In embodiments, the variance at any of these peaks is ±0.1 °2θ.

In embodiments, the monofumarate salt of Compound 1 is characterized by an XRPD having peaks at 7.3±0.2, 10.6±0.2, 13.9±0.2, 18.0±0.2, 19.8±0.2, 20.4±0.2, 23.1±0.2, and 23.6±0.2 °2θ. In embodiments, the variance at any of these peaks is ±0.1 °2θ.

In embodiments, the monofumarate salt of Compound 1 is characterized by an XRPD pattern having one or more peaks at 7.3±0.2, 8.0±0.2, 8.4±0.2, 8.9±0.2, 10.1±0.2, 10.6±0.2, 11.6±0.2, 12.1±0.2, 12.4±0.2, 13.7±0.2, 13.9±0.2, 14.6±0.2, 15.1±0.2, 15.5±0.2, 15.8±0.2, 16.1±0.2, 16.3±0.2, 18.0±0.2, 18.3±0.2, 18.8±0.2, 19.2±0.2, 19.8±0.2, 20.4±0.2, 20.9±0.2, 21.8±0.2, 22.2±0.2, 22.5±0.2, 23.1±0.2, 23.6±0.2, 24.0±0.2, 24.6±0.2, 25.1±0.2, 25.7±0.2, 26.1±0.2, 26.9±0.2, 28.3±0.2, 29.2±0.2, 31.0±0.2, 31.6±0.2, 33.2±0.2, 34.9±0.2, and 36.8±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3L.

In embodiments, monofumarate salt of Compound 1 is characterized by an XRPD pattern comprising peaks shown in Table F.

TABLE F
XRPD Peak Positions for the Monofumarate
Salt of Compound 1 (“Form 6”)

| Peak Position °2θ | Relative Intensity |
|---|---|
| 7.3 | 100.00% |
| 8.0 | 6.05% |
| 8.4 | 8.35% |
| 8.9 | 5.97% |
| 10.1 | 2.72% |
| 10.6 | 66.01% |
| 11.6 | 32.67% |
| 12.1 | 11.78% |
| 12.4 | 11.73% |
| 13.7 | 7.61% |
| 13.9 | 28.93% |
| 14.6 | 5.58% |
| 15.1 | 5.35% |
| 15.5 | 11.30% |
| 15.8 | 18.41% |
| 16.1 | 16.74% |
| 16.3 | 5.10% |
| 18.0 | 30.94% |
| 18.3 | 18.86% |
| 18.8 | 20.43% |
| 19.2 | 22.58% |
| 19.8 | 52.24% |
| 20.4 | 43.15% |
| 20.9 | 25.38% |
| 21.8 | 9.21% |
| 22.2 | 20.58% |
| 22.5 | 27.93% |
| 23.1 | 44.36% |
| 23.6 | 42.49% |
| 24.0 | 33.74% |
| 24.6 | 8.01% |
| 25.1 | 11.15% |
| 25.7 | 19.37% |
| 26.1 | 6.26% |
| 26.9 | 5.22% |
| 28.3 | 4.93% |
| 29.2 | 10.71% |
| 31.0 | 5.21% |
| 31.6 | 4.13% |
| 33.2 | 2.32% |
| 34.9 | 3.02% |
| 36.8 | 2.57% |

In embodiments, monofumarate salt of Compound 1 is characterized by one, two, three, four, five, six, seven, eight, nine, ten, eleven, or more, XRPD peaks listed in Table F.

In embodiments, the monofumarate salt of Compound 1 of Form 6 has a chemical purity of greater than about 80%, about 85%, about 90%, about 95%, about 96%, about 97%, about 98%, about 99%, 99.5%, or about 100% by weight as determined by HPLC.

### Form 7:

In embodiments, the monofumarate salt of Compound 1, is characterized by an XRPD pattern having peaks at 18.7±0.2, 23.6±0.2, and 25.2±0.2 °2θ. In embodiments, the variance at any of these peaks is ±0.1 °2θ.

In embodiments, the monofumarate salt of Compound 1 is characterized by an XRPD having peaks at 18.7±0.2, 23.6±0.2, and 25.2±0.2 °2θ and at least one (e.g., 1, 2, 3, 4, or 5) peak in an XRPD pattern selected from 8.7±0.2, 10.5±0.2, 17.2±0.2, 19.7±0.2, and 21.4±0.2 °2θ. In embodiments, the variance at any of these peaks is ±0.1 °20.

In embodiments, the monofumarate salt of Compound 1 is characterized by an XRPD having peaks at 8.7±0.2, 10.5±0.2, 17.2±0.2, 18.7±0.2, 19.7±0.2, 21.4±0.2, 23.6±0.2, and 25.2±0.2 °2θ. In embodiments, the variance at any of these peaks is ±0.1 °2θ.

In embodiments, the monofumarate salt of Compound 1 is characterized by an XRPD pattern having one or more peaks at 5.3±0.2, 7.2±0.2, 8.7±0.2, 8.9±0.2, 10.2±0.2, 10.5±0.2, 11.5±0.2, 11.8±0.2, 12.2±0.2, 12.8±0.2, 14.1±0.2, 15.2±0.2, 16.5±0.2, 17.2±0.2, 17.9±0.2, 18.2±0.2, 18.7±0.2, 19.4±0.2, 19.7±0.2, 20.8±0.2, 21.1±0.2, 21.4±0.2, 21.9±0.2, 23.2±0.2, 23.6±0.2, 24.0±0.2, 24.9±0.2, 25.2±0.2, 26.4±0.2, 27.5±0.2, 29.2±0.2, 30.3±0.2, and 38.1±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3M.

In embodiments, monofumarate salt of Compound 1 is characterized by an XRPD pattern comprising peaks shown in Table G.

TABLE G
XRPD Peak Positions for the Monofumarate
Salt of Compound 1 (“Form 7”)

| Peak Position °2θ | Relative Intensity |
|---|---|
| 5.3 | 22.31% |
| 7.2 | 31.71% |
| 8.7 | 59.38% |
| 8.9 | 24.91% |
| 10.2 | 34.92% |
| 10.5 | 66.98% |
| 11.5 | 5.99% |
| 11.8 | 11.81% |
| 12.2 | 16.23% |
| 12.8 | 22.05% |
| 14.1 | 21.01% |
| 15.2 | 26.42% |
| 16.5 | 15.17% |
| 17.2 | 80.05% |
| 17.9 | 51.20% |
| 18.2 | 54.23% |
| 18.7 | 90.34% |
| 19.4 | 54.33% |
| 19.7 | 76.74% |
| 20.8 | 12.45% |
| 21.1 | 32.13% |
| 21.4 | 75.17% |
| 21.9 | 48.60% |
| 23.2 | 67.15% |
| 23.6 | 100.00% |
| 24.0 | 27.37% |
| 24.9 | 25.35% |
| 25.2 | 86.24% |
| 26.4 | 25.54% |
| 27.5 | 6.78% |
| 29.2 | 15.66% |
| 30.3 | 8.66% |
| 38.1 | 6.21% |

In embodiments, monofumarate salt of Compound 1 is characterized by one, two, three, four, five, six, seven, eight, nine, ten, eleven, or more, XRPD peaks listed in Table G.

In embodiments, the monofumarate salt of Compound 1 of Form 7 has a chemical purity of greater than about 80%, about 85%, about 90%, about 95%, about 96%, about 97%, about 98%, about 99%, 99.5%, or about 100% by weight as determined by HPLC.

### Form 8:

In embodiments, the monofumarate salt of Compound 1, is characterized by an XRPD pattern having peaks at 5.6±0.2, 11.2±0.2, and 19.7±0.2 °2θ. In embodiments, the variance at any of these peaks is ±0.1 °2θ.

In embodiments, the monofumarate salt of Compound 1 is characterized by an XRPD having peaks at 5.6±0.2, 11.2±0.2, and 19.7±0.2 °2θ and at least one (e.g., 1, 2, 3, 4, or 5) peak in an XRPD pattern selected from 9.3±0.2, 16.8±0.2, 18.6±0.2, 21.1±0.2, and 23.5±0.2 °2θ. In embodiments, the variance at any of these peaks is ±0.1 °2θ.

In embodiments, the monofumarate salt of Compound 1 is characterized by an XRPD having peaks at 5.6±0.2, 9.3±0.2, 11.2±0.2, 16.8±0.2, 18.6±0.2, 19.7±0.2, 21.1±0.2, and 23.5±0.2 °2θ. In embodiments, the variance at any of these peaks is ±0.1 °20.

In embodiments, the monofumarate salt of Compound 1 is characterized by an XRPD pattern having one or more peaks at 5.6±0.2, 7.1±0.2, 8.1±0.2, 8.7±0.2, 9.3±0.2, 10.4±0.2, 11.2±0.2, 12.7±0.2, 13.2±0.2, 14.4±0.2, 15.0±0.2, 15.8±0.2, 16.8±0.2, 18.6±0.2, 19.3±0.2, 19.7±0.2, 20.8±0.2, 21.1±0.2, 21.8±0.2, 22.5±0.2, 23.2±0.2, 23.5±0.2, 25.1±0.2, 29.1±0.2, and 29.5±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3N.

In embodiments, monofumarate salt of Compound 1 is characterized by an XRPD pattern comprising peaks shown in Table H.

TABLE H
XRPD Peak Positions for the Monofumarate
Salt of Compound 1 (“Form 8”)

| Peak Position °2θ | Relative Intensity |
|---|---|
| 5.6 | 88.19% |
| 7.1 | 3.37% |
| 8.1 | 14.51% |
| 8.7 | 12.42% |
| 9.3 | 19.45% |
| 10.4 | 4.86% |
| 11.2 | 100.00% |
| 12.7 | 7.25% |
| 13.2 | 6.64% |
| 14.4 | 3.39% |
| 15.0 | 3.65% |
| 15.8 | 9.17% |
| 16.8 | 24.21% |
| 18.6 | 19.48% |
| 19.3 | 18.39% |
| 19.7 | 30.58% |
| 20.8 | 5.96% |
| 21.1 | 20.26% |
| 21.8 | 5.52% |
| 22.5 | 3.58% |
| 23.2 | 8.38% |
| 23.5 | 21.51% |
| 25.1 | 10.17% |
| 29.1 | 2.58% |
| 29.5 | 3.34% |

In embodiments, monofumarate salt of Compound 1 is characterized by one, two, three, four, five, six, seven, eight, nine, ten, eleven, or more, XRPD peaks listed in Table H.

In embodiments, the monofumarate salt of Compound 1 of Form 8 has a chemical purity of greater than about 80%, about 85%, about 90%, about 95%, about 96%, about 97%, about 98%, about 99%, 99.5%, or about 100% by weight as determined by HPLC.

### Form 9:

In embodiments, the monofumarate salt of Compound 1, is characterized by an XRPD pattern having peaks at 10.8±0.2, 16.2±0.2, and 21.6±0.2 °2θ. In embodiments, the variance at any of these peaks is ±0.1 °2θ.

In embodiments, the monofumarate salt of Compound 1 is characterized by an XRPD having peaks at 10.8±0.2, 16.2±0.2, and 21.6±0.2 °2θ and at least one (e.g., 1, 2, 3, 4, or 5) peak in an XRPD pattern selected from 5.5±0.2, 12.1±0.2, 17.7±0.2, 18.2±0.2, and 22.7±0.2 °2θ. In embodiments, the variance at any of these peaks is ±0.1 °2θ.

In embodiments, the monofumarate salt of Compound 1 is characterized by an XRPD having peaks at 5.5±0.2, 10.8±0.2, 12.1±0.2, 16.2±0.2, 17.7±0.2, 18.2±0.2, 21.6±0.2, and 22.7±0.2 °2θ. In embodiments, the variance at any of these peaks is ±0.1 °20.

In embodiments, the monofumarate salt of Compound 1 is characterized by an XRPD pattern having one or more peaks at 5.5±0.2, 6.1±0.2, 8.2±0.2, 8.8±0.2, 9.6±0.2, 10.0±0.2, 10.8±0.2, 11.4±0.2, 12.1±0.2, 12.9±0.2, 13.2±0.2, 14.6±0.2, 16.2±0.2, 17.0±0.2, 17.7±0.2, 18.2±0.2, 19.1±0.2, 19.9±0.2, 20.4±0.2, 20.7±0.2, 21.6±0.2, 22.7±0.2, 23.5±0.2, 24.2±0.2, 25.3±0.2, 27.9±0.2, 28.6±0.2, 29.5±0.2, 32.5±0.2, and 33.3±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3O.

In embodiments, monofumarate salt of Compound 1 is characterized by an XRPD pattern comprising peaks shown in Table I.

TABLE I
XRPD Peak Positions for the Monofumarate
Salt of Compound 1 (“Form 9”)

| Peak Position °20 | Relative Intensity |
|---|---|
| 5.5 | 16.11% |
| 6.1 | 1.13% |
| 8.2 | 3.71% |
| 8.8 | 1.57% |
| 9.6 | 1.45% |
| 10.0 | 3.67% |
| 10.8 | 48.16% |
| 11.4 | 6.55% |
| 12.1 | 10.24% |
| 12.9 | 2.55% |
| 13.2 | 1.42% |
| 14.6 | 1.34% |
| 16.2 | 100.00% |
| 17.0 | 7.88% |
| 17.7 | 11.69% |
| 18.2 | 14.50% |
| 19.1 | 1.64% |
| 19.9 | 8.68% |
| 20.4 | 5.27% |
| 20.7 | 3.44% |
| 21.6 | 36.48% |
| 22.7 | 17.27% |
| 23.5 | 4.20% |
| 24.2 | 4.25% |
| 25.3 | 2.97% |
| 27.9 | 3.10% |
| 28.6 | 1.31% |
| 29.5 | 0.61% |
| 32.5 | 2.75% |
| 33.3 | 1.28% |

In embodiments, monofumarate salt of Compound 1 is characterized by one, two, three, four, five, six, seven, eight, nine, ten, eleven, or more, XRPD peaks listed in Table I.

In embodiments, the monofumarate salt of Compound 1 of Form 9 has a chemical purity of greater than about 80%, about 85%, about 90%, about 95%, about 96%, about 97%, about 98%, about 99%, 99.5%, or about 100% by weight as determined by HPLC.

In embodiments, the liquid formulation of the present disclosure comprises a sesquifumarate salt of Compound 1. In embodiments, the sesquifumarate salt of Compound 1 has the following formula:

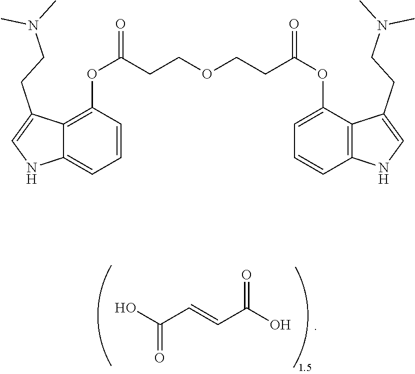

In embodiments, the sesquifumarate salt of Compound 1 is crystalline.

In embodiments, the sesquifumarate salt of Compound 1 is characterized by an XRPD pattern having peaks at 8.7±0.2, 19.7±0.2, and 23.6±0.2 °2θ.

In embodiments, the sesquifumarate salt of Compound 1 is characterized by an XRPD pattern having peaks at 8.7±0.2, 19.7±0.2, and 23.6±0.2 °2θ and at least one (e.g., 1, 2, or 3) peak in an XRPD pattern selected from 14.4±0.2, 21.8±0.2, and 22.2±0.2 °2θ. In embodiments, the variance at any of these peaks is ±0.1 °2θ.

In embodiments, the sesquifumarate salt of Compound 1 is characterized by an XRPD pattern having peaks at 8.7±0.2, 14.4±0.2, 19.7±0.2, 21.8±0.2, 22.2±0.2, 23.6±0.2 °2θ. In embodiments, the variance at any of these peaks is ±0.1 °2θ.

In embodiments, the sesquifumarate salt of Compound 1 is characterized by an XRPD pattern having one or more peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °20. In embodiments, the sesquifumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, or 26 of the aforementioned peaks, or any range therein (e.g., from 1-26, 2-20, 3-15, or 3-10 of the aforementioned peaks).

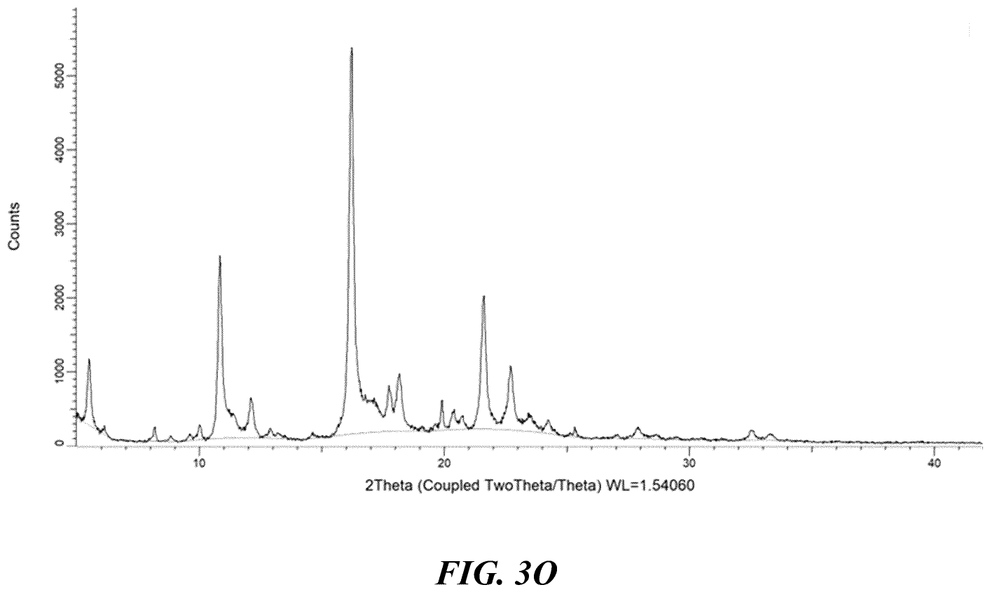

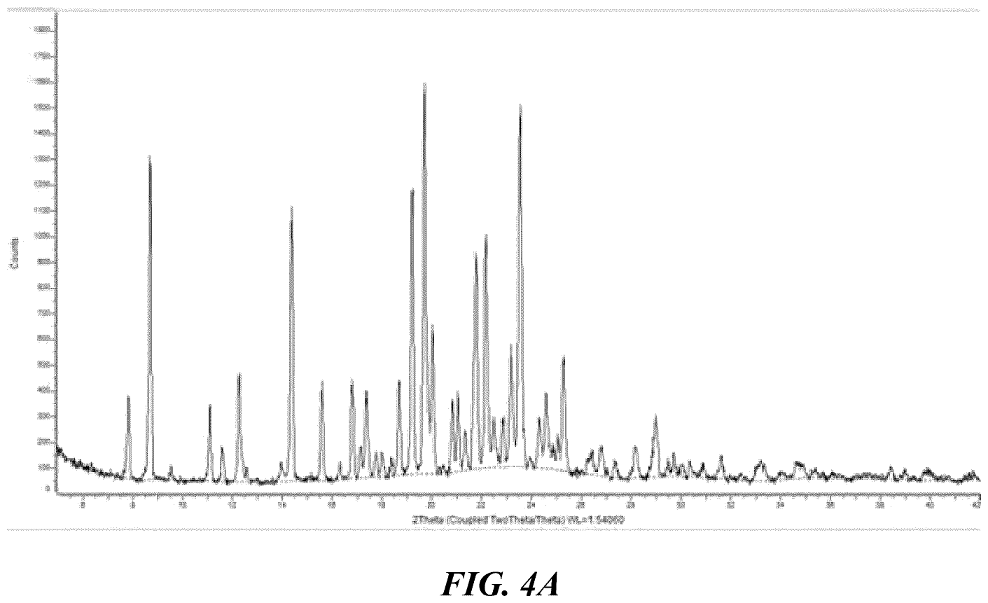

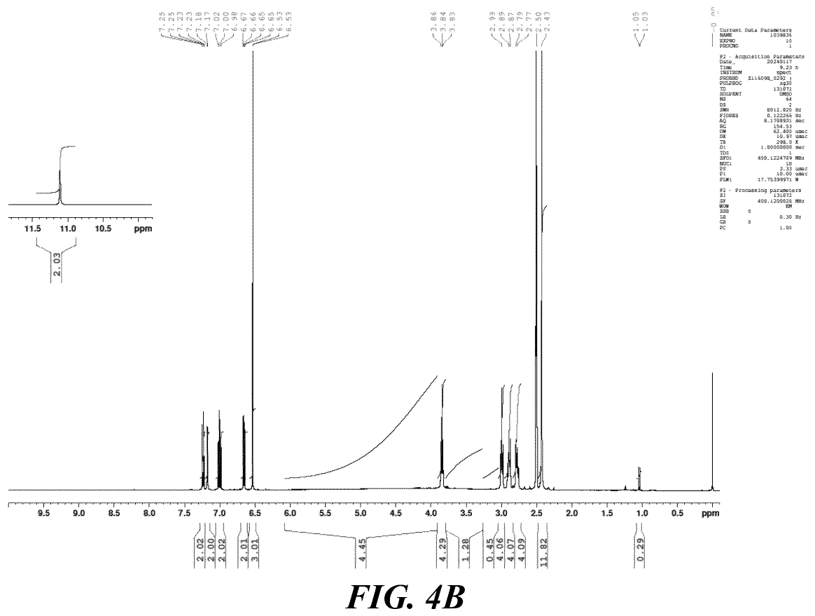

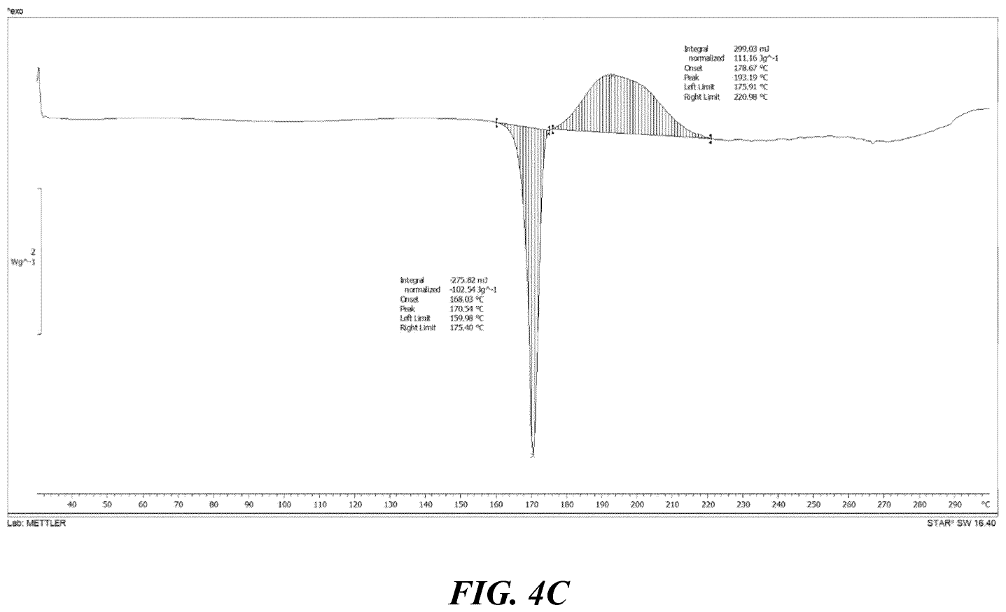

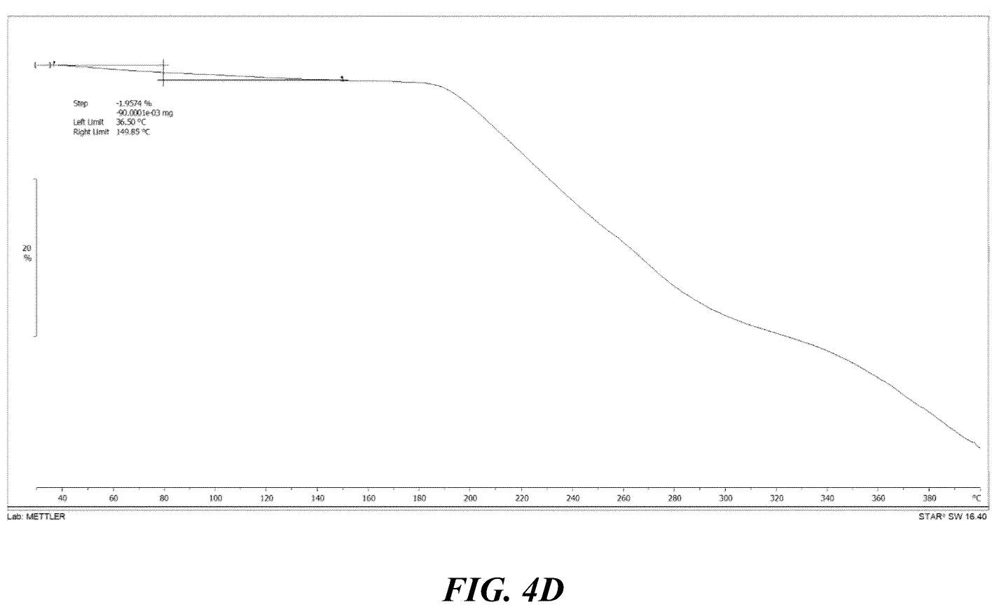

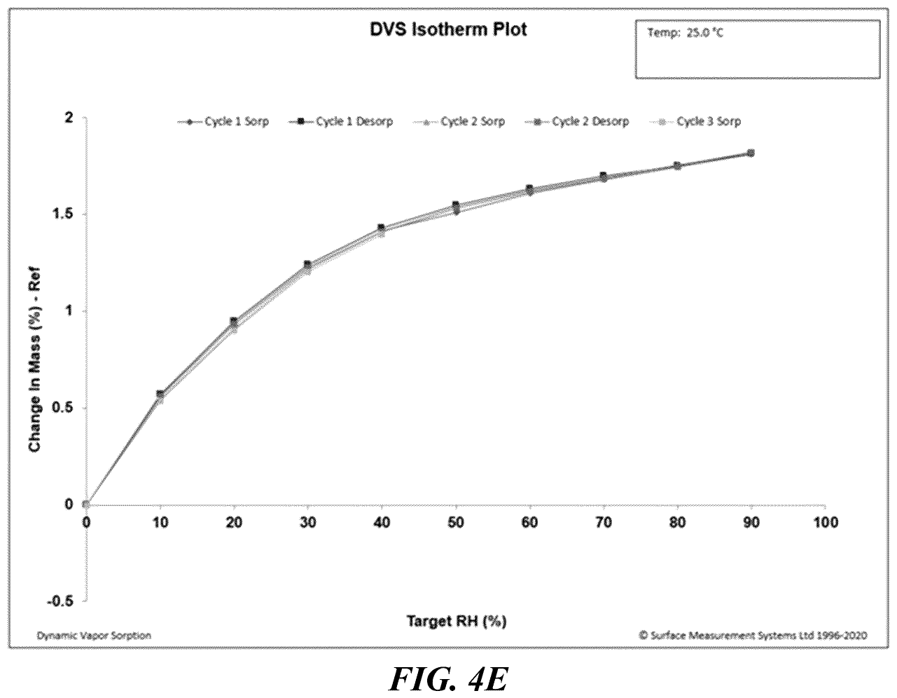

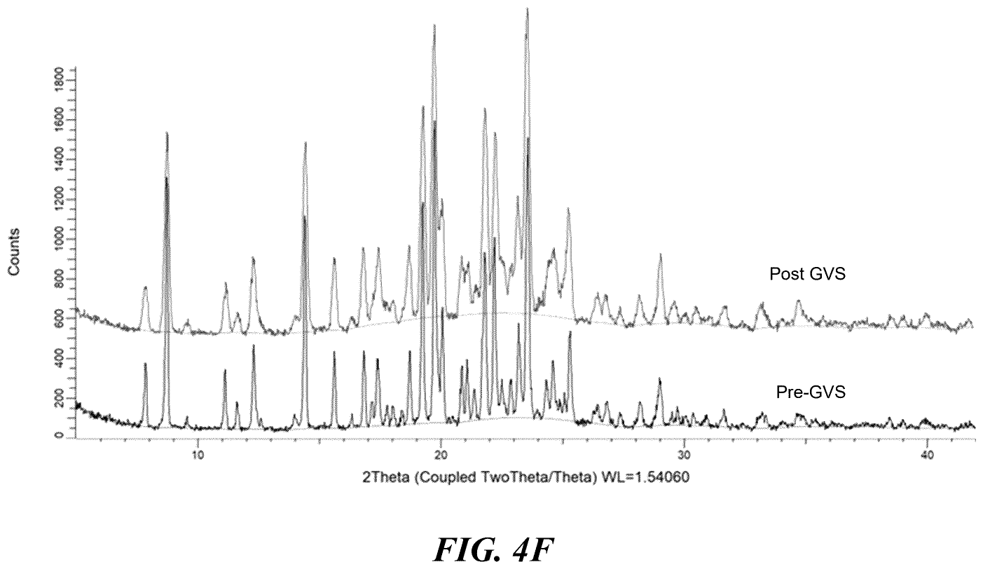

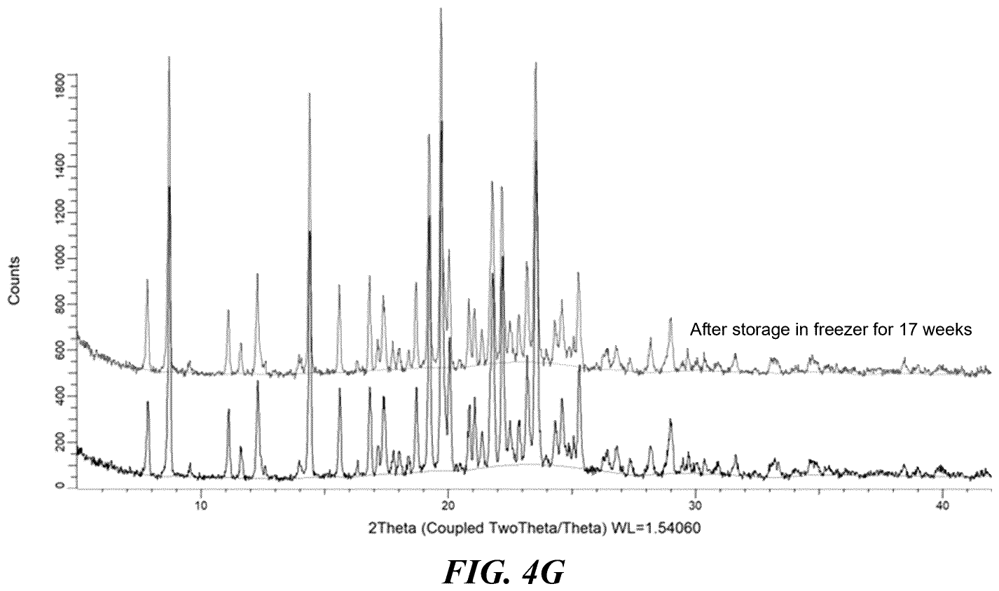

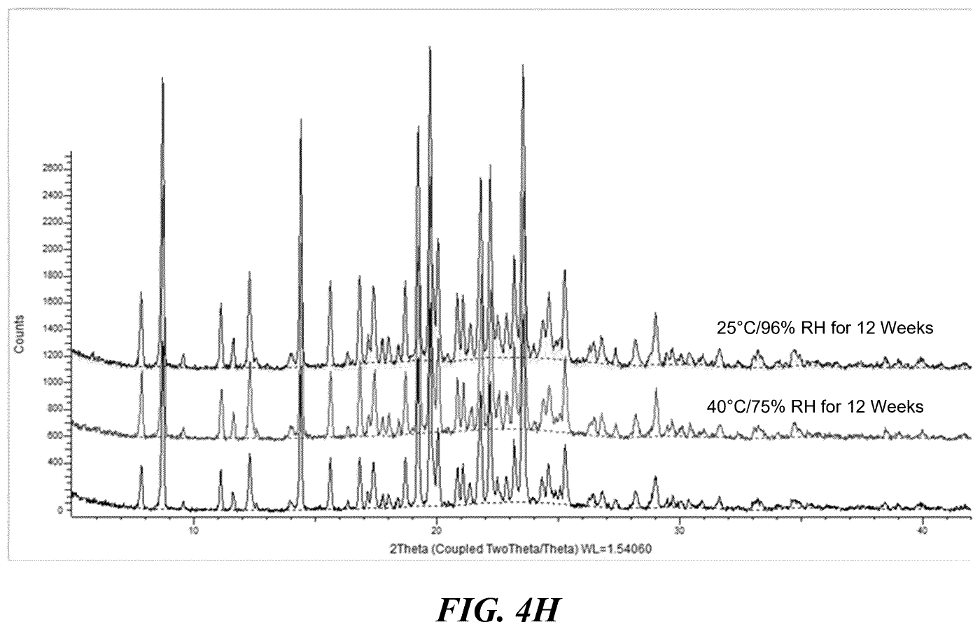

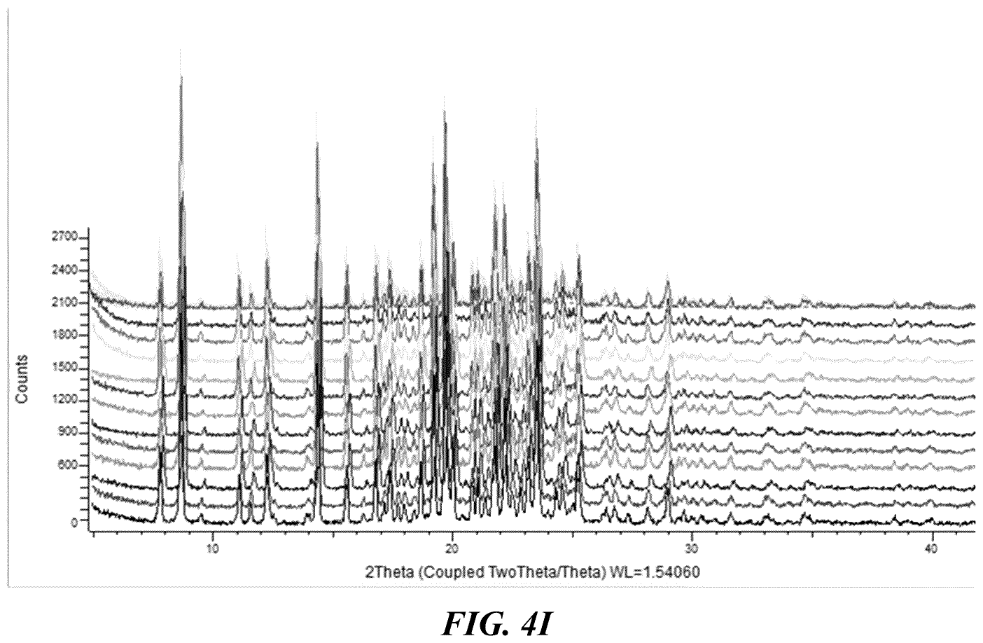

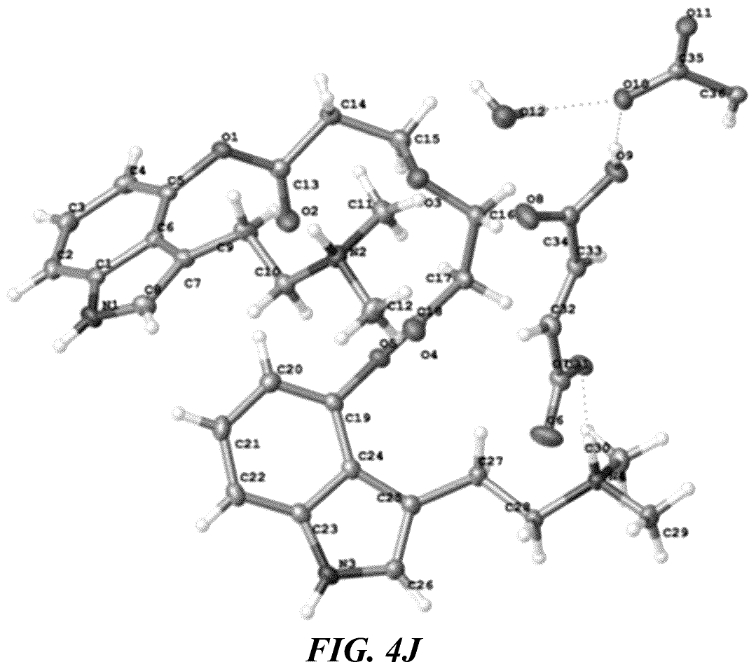

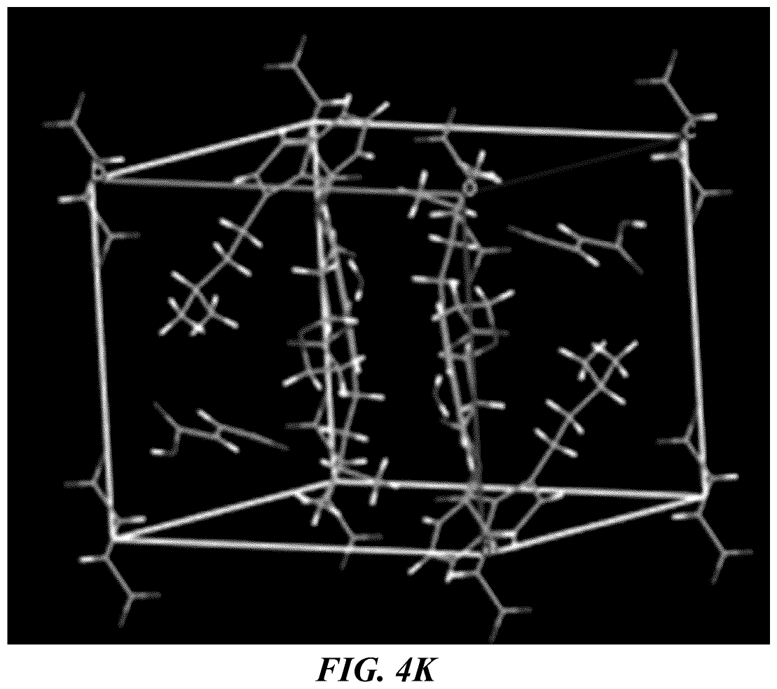

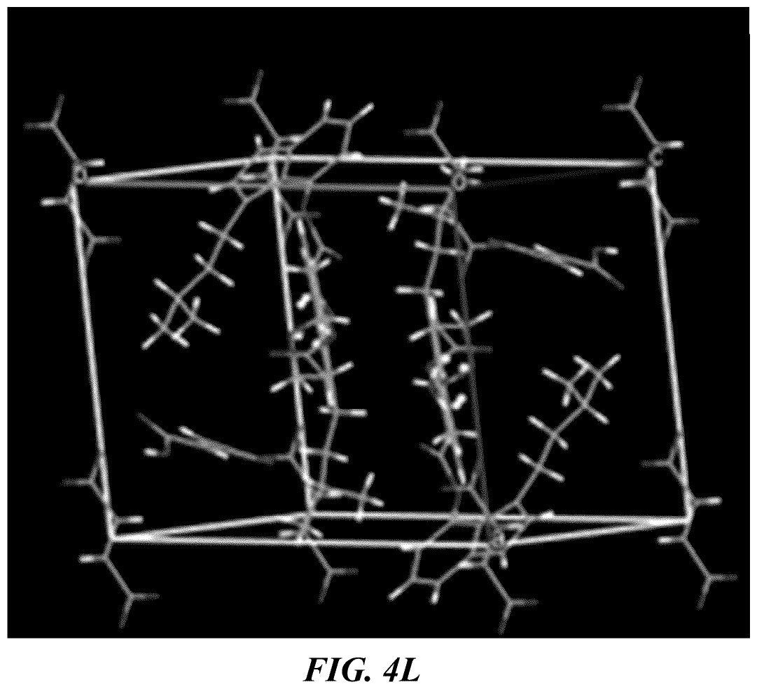

In embodiments, the sesquifumarate salt of Compound 1 is characterized by an XRPD pattern substantially similar to FIG. 4A.

In embodiments, sesquifumarate salt of Compound 1 is characterized by an XRPD pattern comprising peaks shown in Table J.

TABLE J
XRPD Peak Positions for the Sesquifumarate Salt of Compound 1

| Position°2θ | Relative Intensity | Position°2θ | Relative Intensity |
|---|---|---|---|
| 7.9 | 18.1% | 22.9 | 12.6% |
| 8.7 | 76.9% | 23.2 | 30.5% |
| 9.6 | 3.5% | 23.6 | 85.6% |
| 11.1 | 18.4% | 24.3 | 12.3% |
| 11.6 | 7.2% | 24.6 | 20.3% |
| 12.3 | 26.4% | 25.3 | 28.1% |
| 14.0 | 4.8% | 26.4 | 5.9% |
| 14.4 | 66.2% | 26.8 | 7.6% |
| 15.6 | 24.7% | 27.4 | 3.0% |
| 16.3 | 4.6% | 28.2 | 8.2% |
| 16.8 | 24.5% | 29.0 | 14.2% |
| 17.2 | 7.3% | 29.5 | 4.3% |
| 17.4 | 18.1% | 29.7 | 6.8% |
| 17.8 | 6.8% | 30.1 | 3.4% |
| 18.0 | 6.1% | 30.4 | 5.7% |
| 18.4 | 5.4% | 30.9 | 2.1% |
| 18.7 | 24.5% | 31.6 | 7.0% |
| 19.2 | 74.0% | 33.2 | 5.9% |
| 19.7 | 100.0% | 34.0 | 2.5% |
| 20.1 | 38.7% | 34.7 | 2.9% |
| 20.8 | 18.2% | 34.9 | 3.0% |
| 21.1 | 21.9% | 35.4 | 2.3% |
| 21.4 | 10.7% | 38.4 | 3.9% |
| 21.8 | 55.6% | 39.0 | 2.7% |
| 22.2 | 60.2% | 39.9 | 1.5% |
| 22.5 | 13.4% | | |

In embodiments, sesquifumarate salt of Compound 1 is characterized by one, two, three, four, five, six, seven, eight, nine, ten, eleven, or more, XRPD peaks listed in Table J.

In embodiments, the sesquifumarate salt of Compound 1 is characterized by a DSC thermogram comprising an endothermic event at 168±5° C.

In embodiments, the sesquifumarate salt of Compound 1 is characterized by a DSC comprising an exothermic event between 176±5° C. to 221±5° C.

In embodiments, the sesquifumarate salt of Compound 1 is characterized by a DSC thermogram substantially similar to FIG. 4C.

In embodiments, the sesquifumarate salt of Compound 1 is characterized by about a 2% weight loss from 37±5° C. to 150±5° C. as determined by TGA.

In embodiments, the sesquifumarate salt of Compound 1 is characterized by a thermogravimetric thermogram substantially similar to FIG. 4D.

In embodiments, the sesquifumarate salt of Compound 1 has a chemical purity of greater than about 80%, about 85%, about 90%, about 95%, about 96%, about 97%, about 98%, about 99%, 99.5%, or about 100% by weight as determined by HPLC.

In embodiments, the sesquifumarate salt of Compound 1 is a hydrate.

In embodiments, the liquid formulation of the present disclosure comprises a succinate salt of Compound 1. In embodiments, the succinate salt of Compound 1 has the following formula:

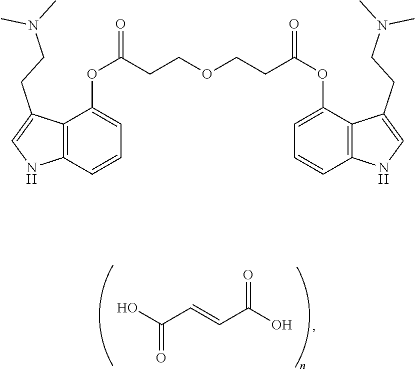

wherein n is from 1 to 3.

In embodiments, n is 1.

In embodiments, n is 1.5.

In embodiments, n is 2.

In embodiments, n is 2.5.

In embodiments, n is 3.

In embodiments, succinate salt of Compound 1 is crystalline.

In embodiments, succinate salt of Compound 1 is characterized by an XRPD pattern having peaks at 19.7±0.2, 22.1±0.2, and 23.1±0.2 °2θ. In embodiments, the variance at any of these peaks is ±0.1 °2θ.

In embodiments, the succinate salt of Compound 1 is characterized by an XRPD pattern having peaks at 19.7±0.2, 22.1±0.2, and 23.1±0.2 °2θ and at least one (e.g., 1, 2, 3, 4, 5, or 6) peak in an XRPD pattern selected from 8.7±0.2, 14.3±0.2, 16.8±0.2, 18.6±0.2, 19.0±0.2, and 20.2±0.2 °2θ. In embodiments, the variance at any of these peaks is ±0.1 °2θ.

In embodiments, the succinate salt of Compound 1 is characterized by an XRPD pattern having peaks at 8.7±0.2, 14.3±0.2, 16.8±0.2, 18.6±0.2, 19.0±0.2, 19.7±0.2, 20.2±0.2 22.1±0.2, and 23.1±0.2 °2θ. In embodiments, the variance at any of these peaks is ±0.1 °2θ.

In embodiments, the succinate salt of Compound 1 is characterized by an XRPD pattern having one or more peaks at 7.8±0.2, 8.7±0.2, 9.7±0.2, 10.8±0.2, 11.6±0.2, 12.0±0.2, 12.5±0.2, 13.9±0.2, 14.3±0.2, 15.6±0.2, 15.9±0.2, 16.5±0.2, 16.8±0.2, 17.4±0.2, 18.0±0.2, 18.6±0.2, 19.0±0.2, 19.7±0.2, 20.2±0.2, 20.7±0.2, 21.2±0.2, 21.7±0.2, 22.1±0.2, 22.3±0.2, 23.1±0.2, 23.6±0.2, 23.9±0.2, 24.6±0.2, 24.9±0.2, 25.3±0.2, 26.2±0.2, 26.9±0.2, 27.6±0.2, 28.1±0.2, 28.3±0.2, 28.9±0.2, 29.3±0.2, 30.2±0.2, 30.4±0.2, 33.2±0.2, 34.6±0.2, 35.1±0.2, 35.7±0.2, 38.0±0.2, 38.7±0.2, and 39.3±0.2 and °2θ. In embodiments, the variance at any of these peaks is ±0.1 °2θ. In embodiments, the succinate salt of Compound 1 may be characterized by 1, 2, 3, 4, 5, 6, 7, 8, 9, 10, 11, 12, 13, 14, 15, 16, 16, 17, 18, 19, 20, 21, 22, or 23 of the aforementioned peaks, or any range therein (e.g., from 1-23, 2-20, 3-15, or 3-10 of the aforementioned peaks).

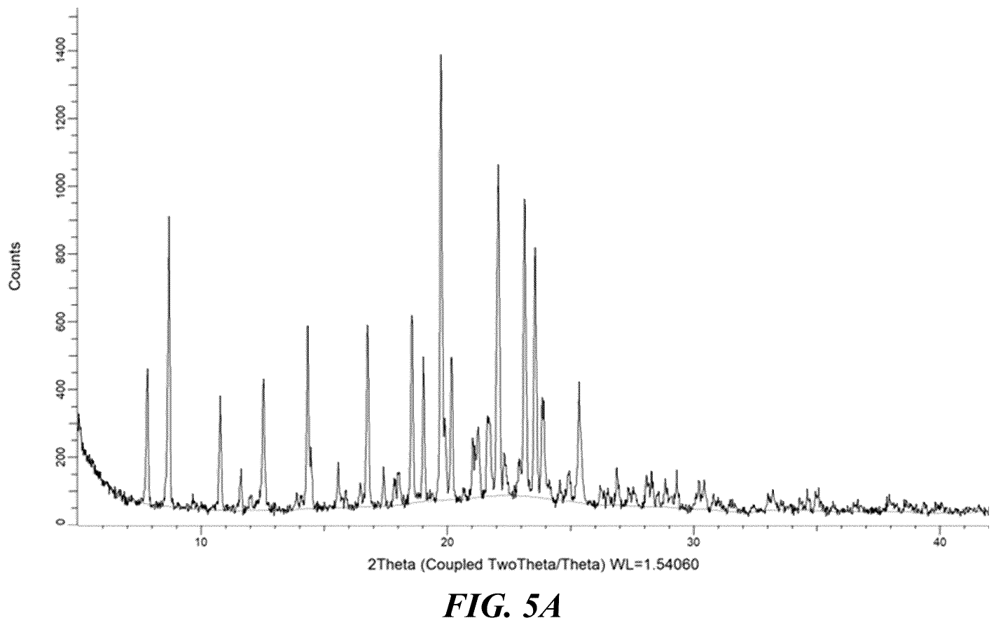

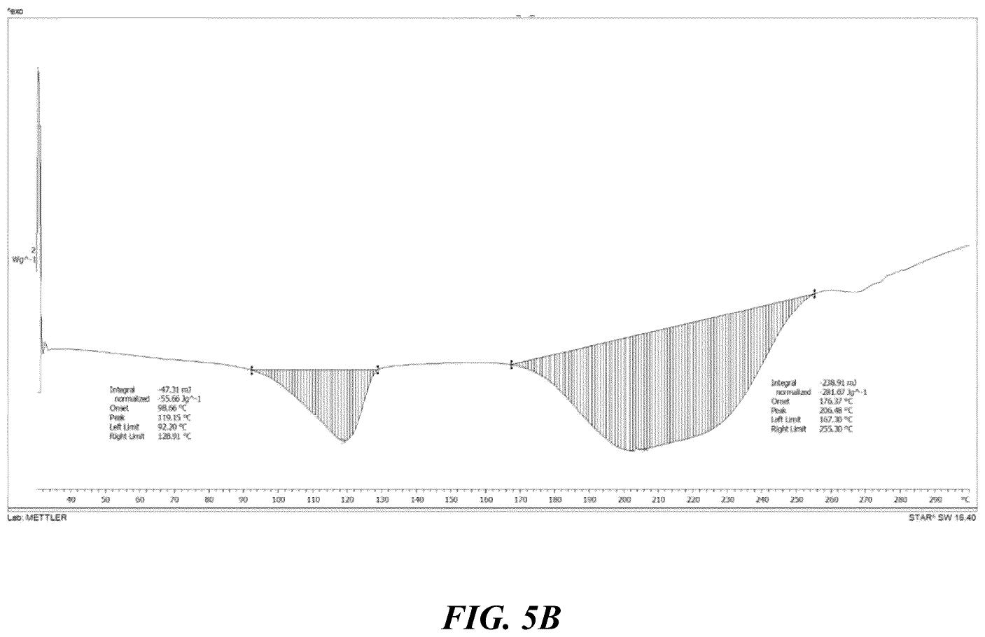

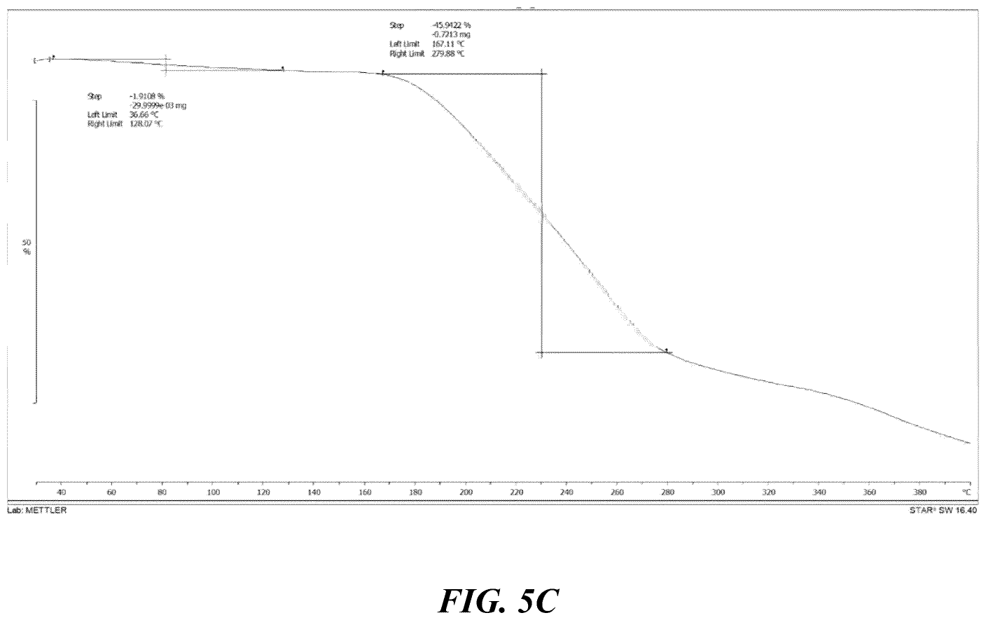

In embodiments, the succinate salt of Compound 1 is characterized by an XRPD pattern substantially similar to FIG. 5A.

In embodiments, succinate salt of Compound 1 is characterized by an XRPD pattern comprising peaks shown in Table K.

TABLE K
XRPD Peak Positions for the Succinate Salt of Compound 1

| | Relative | | |
|---|---|---|---|
| Peak position°2θ | Intensity | Peak position°2θ | Relative Intensity |
| 7.8 | 26.4% | 22.3 | 8.2% |
| 8.7 | 58.2% | 23.1 | 69.8% |
| 9.7 | 1.5% | 23.6 | 54.4% |
| 10.8 | 24.1% | 23.9 | 23.4% |
| 11.6 | 7.9% | 24.6 | 5.1% |
| 12.0 | 2.7% | 24.9 | 5.4% |
| 12.5 | 26.1% | 25.3 | 31.3% |
| 13.9 | 3.9% | 26.2 | 3.9% |
| 14.3 | 39.1% | 26.9 | 9.2% |
| 15.6 | 10.9% | 27.6 | 4.8% |
| 15.9 | 3.3% | 28.1 | 7.9% |
| 16.5 | 5.1% | 28.3 | 9.2% |
| 16.8 | 39.2% | 28.9 | 6.2% |
| 17.4 | 8.9% | 29.3 | 9.6% |
| 18.0 | 6.5% | 30.2 | 7.2% |
| 18.6 | 40.6% | 30.4 | 6.7% |
| 19.0 | 31.9% | 33.2 | 5.2% |
| 19.7 | 100.0% | 34.6 | 4.6% |
| 20.2 | 31.2% | 35.1 | 3.7% |
| 20.7 | 2.1% | 35.7 | 2.1% |
| 21.2 | 15.7% | 38.0 | 3.2% |
| 21.7 | 14.3% | 38.7 | 1.5% |
| 22.1 | 67.9% | 39.3 | 2.5% |

In embodiments, succinate salt of Compound 1 is characterized by one, two, three, four, five, six, seven, eight, nine, ten, eleven, or more, XRPD peaks listed in Table K.

In embodiments, the succinate salt of Compound 1 is characterized by a DSC thermogram comprising an endothermic event between 92±5° C. to 129±5° C.

In embodiments, the succinate salt of Compound 1 is characterized by a Differential Scanning calorimetry (DSC) thermogram comprising an endothermic onset at 99±5° C.

In embodiments, the succinate salt of Compound 1 is characterized by a DSC thermogram substantially similar to FIG. 5B.

In embodiments, the succinate salt of Compound 1 is characterized by about a 2% weight loss from 37±5° C. to 128±5° C. as determined by thermal gravimetric analysis (TGA).

In embodiments, the succinate salt of Compound 1 is characterized by a thermogravimetric thermogram substantially similar to FIG. 5C.

In embodiments, the succinate salt of Compound 1 has a chemical purity of greater than about 80%, about 85%, about 90%, about 95%, about 96%, about 97%, about 98%, about 99%, 99.5%, or about 100% by weight as determined by HPLC.

In embodiments, the liquid formulation of the present disclosure comprises a tartrate salt of Compound 1. In embodiments, the liquid formulation of the present disclosure comprises a L-tartrate salt of Compound 1.

In embodiments, the L-tartrate salt of Compound 1 is crystalline.

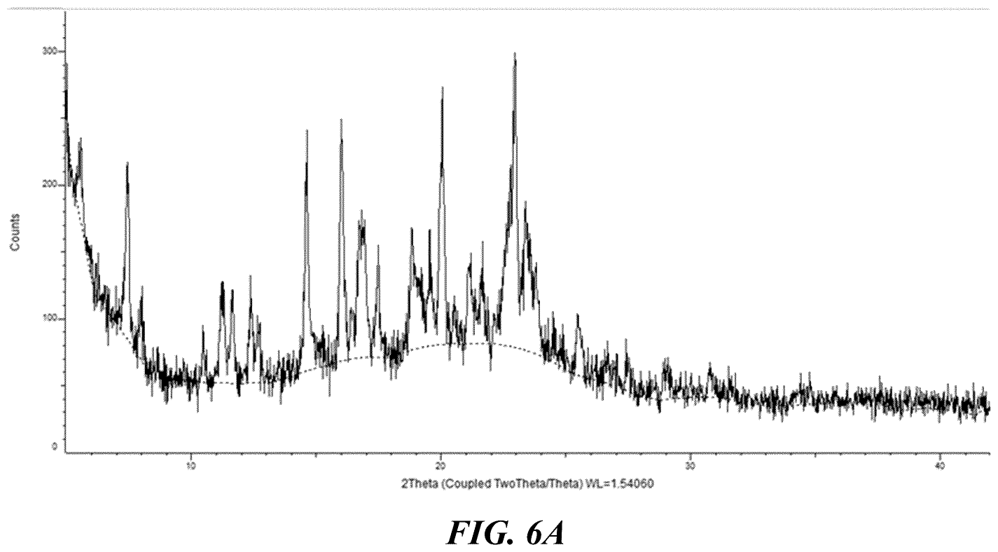

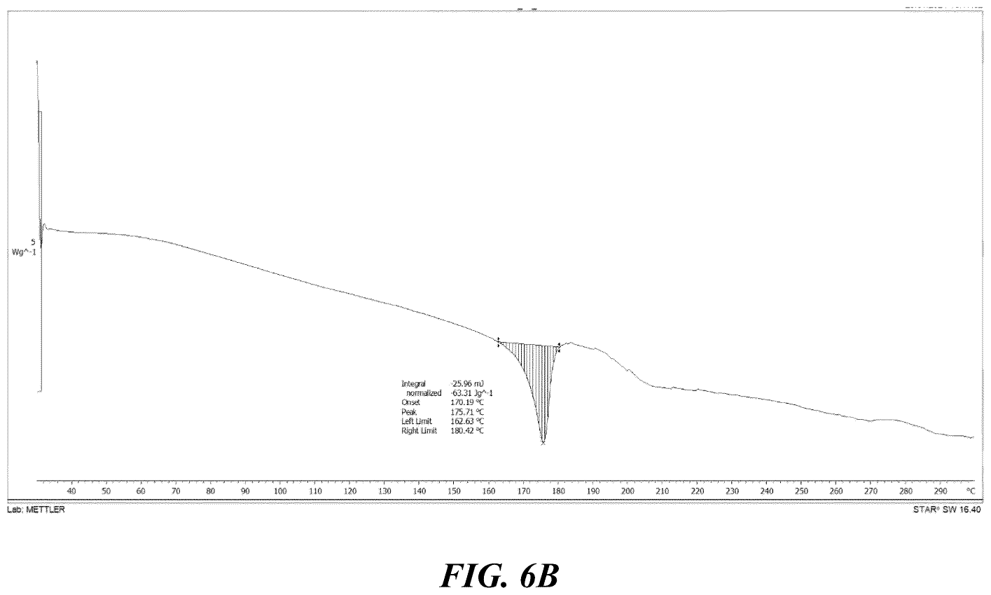

In embodiments, the L-tartrate salt of Compound 1 is characterized by an XRPD pattern substantially similar to FIG. 6A.

In embodiments, the L-tartrate salt of Compound 1 is characterized by a DSC thermogram comprising an endothermic onset at 170±5° C.

In embodiments, the L-tartrate salt of Compound 1 is characterized by a DSC thermogram substantially similar to FIG. 6B.

In embodiments, the L-tartrate salt of Compound 1 has a chemical purity of greater than about 80%, about 85%, about 90%, about 95%, about 96%, about 97%, about 98%, about 99%, 99.5%, or about 100% by weight as determined by HPLC.

In embodiments, the liquid formulation of the present disclosure comprises a fumarate salt of Compound 1. In embodiments, the fumarate salt of Compound 1 has the following chemical formula:

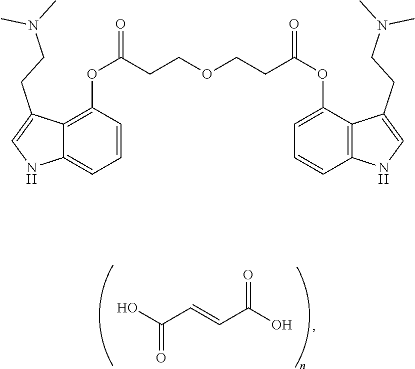

wherein n ranges from 1 to 3.

In embodiments, n is 1.

In embodiments, n is 1.5.

In embodiments, n is 2.

In embodiments, n is 2.5.

In embodiments, n is 3.

In embodiments, the fumarate salt of Compound 1 has the following chemical formula:

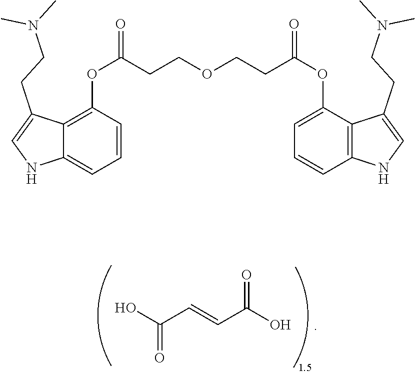

Such a fumarate salt is referred to herein as a sesquifumarate salt.

In embodiments, the sesquifumarate salt is characterized by an XRPD pattern having peaks at 8.7±0.2, 19.7±0.2, and 23.6±0.2 °2θ. In embodiments, the fumarate salt is further characterized by peaks in an XRPD pattern at 14.4±0.2, 21.8±0.2, and 22.2±0.2 °2θ.

In embodiments, the fumarate salt of Compound 1 has the following chemical formula:

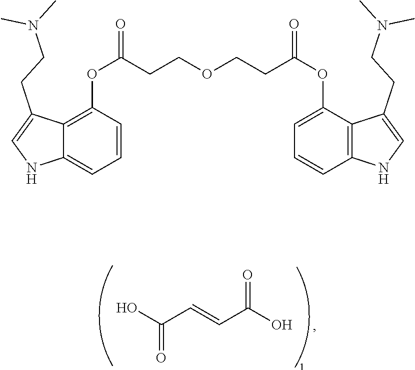

wherein characterized by an XRPD pattern having peaks at 12.8±0.2, 18.7±0.2, and 23.2±0.2 °2θ. In embodiments, the fumarate salt is further characterized by peaks in an XRPD pattern at 7.2±0.2, 10.4±0.2, 17.9±0.2, 19.3±0.2, and 21.4±0.2 °2θ.

In embodiments, the liquid formulation of the present disclosure comprises a bis-formate salt of Compound 1.

In embodiments, the liquid formulation comprises 0.1 mg/ml to about 500 mg/ml of Compound 1, or a pharmaceutically acceptable salt thereof (e.g., the salts disclosed herein), relative to a total volume of the formulation. In embodiments, the liquid formulation comprises 0.1 mg/mL to about 500 mg/mL, 0.5 mg/ml to 250 mg/mL, 1 mg/mL to 100 mg/mL, 2 mg/mL to 50 mg/mL, 2.5 mg/mL to 40 mg/mL, 4 mg/ml to 30 mg/mL, or 5 mg/mL to 25 mg/mL of Compound 1, or a pharmaceutically acceptable salt thereof, relative to a total volume of the formulation.

In embodiments, the liquid formulation comprises about 2 mg/ml to about 40 mg/mL, about 5 mg/mL to about 35 mg/mL, about 10 mg/mL to about 30 mg/mL, or about 15 mg/mL to about 20 mg/ml of Compound 1, or an equivalent amount of a pharmaceutically acceptable salt thereof, relative to a total volume of the formulation. In embodiments, the formulation comprises about 30 mg/mL to about 40 mg/mL, about 32 mg/mL to about 38 mg/mL, or about 34 mg/mL to about 36 mg/mL of Compound 1, or an equivalent amount of a pharmaceutically acceptable salt thereof, relative to a total volume of the formulation.

In embodiments, the pharmaceutically acceptable salt forms of Compound 1 may be present in the formulation in an amount of about 2-40 mg/mL. In embodiments, the pharmaceutically acceptable salt forms of Compound 1 may be present in the formulation in an amount of about 2-30 mg/mL, about 15-30 mg/mL, about 22.5 mg/mL, about 25 mg/mL, about 27.5 mg/mL, or about 30 mg/mL, or about 40 mg/ml. The concentrations of the salt of Compound 1 here are determined based on the weight of an equivalent amount of Compound 1 (as a free base).

In embodiments, the liquid formulation comprises about 30 mg/ml of pharmaceutically acceptable salt of Compound 1 relative to a total volume of the formulation. In embodiments, the liquid formulation comprises about 40 mg/mL of pharmaceutically acceptable salt of Compound 1 relative to a total volume of the formulation. The pharmaceutically acceptable salt form of Compound 1 may be a sesquifumerate salt of Compound 1 and may be present in the formulation in an amount of 30 mg/mL. The concentrations of the salt of Compound 1 here are determined based on the weight of an equivalent amount of Compound 1 (as a free base).

In embodiments, to facilitate efficient dissolution of Compound 1, the liquid formulation comprises less than 40 mg/mL, less than 35 mg/mL, or less than 32 mg/mL Compound 1 (as a free base), relative to a total volume of the formulation.

In embodiments, the pharmaceutically acceptable salt form of Compound 1 has a chemical purity of greater than about 90%, by weight, as determined by HPLC analysis. In embodiments, the pharmaceutically acceptable salt form of Compound 1 has a chemical purity of greater than about 95%, by weight, as determined by HPLC analysis. In embodiments, the pharmaceutically acceptable salt form of Compound 1 has a chemical purity of greater than about 99.5%, by weight, as determined by HPLC analysis. The pharmaceutically acceptable salt form of Compound 1 may be a monofumarate salt, a sesquifumerate salt, a succinate salt, or an L-tartrate salt having a purity of greater than about 90% to greater than about 99.5%.

### Excipients

The liquid formulations also comprise one or more pharmaceutically acceptable excipients, which can facilitate delivery (e.g., parenteral administration, in particular subcutaneous injection) of Compound 1 or the salts of Compound 1 disclosed herein in any of its embodiments to a subject in need thereof. Other purposes of the formulations comprising the pharmaceutically acceptable excipients are to enhance dispersion, solubility, and stability of Compound 1, and to reduce adverse injection site reactions.

The present disclosure provides liquid formulations comprising Compound 1, or a pharmaceutically acceptable salt thereof (e.g., monofumarate salt, a sesquifumarate salt, bis-formate salt, succinate salt, or L-tartrate salt of Compound 1) disclosed herein and one or more pharmaceutically acceptable excipients.

In embodiments, the liquid formulation comprises Compound 1 as a free base and one or more pharmaceutically acceptable excipients.

In embodiments, the liquid formulation comprises a pharmaceutically acceptable salt of Compound 1 and one or more pharmaceutically acceptable excipients.

In embodiments, the liquid formulation comprises a monofumarate salt of Compound 1 and one or more pharmaceutically acceptable excipients.

In embodiments, the liquid formulation comprises a sesquifumarate salt of Compound 1 and one or more pharmaceutically acceptable excipients.

In embodiments, the liquid formulation comprises a bis-formate salt of Compound 1 and one or more pharmaceutically acceptable excipients.

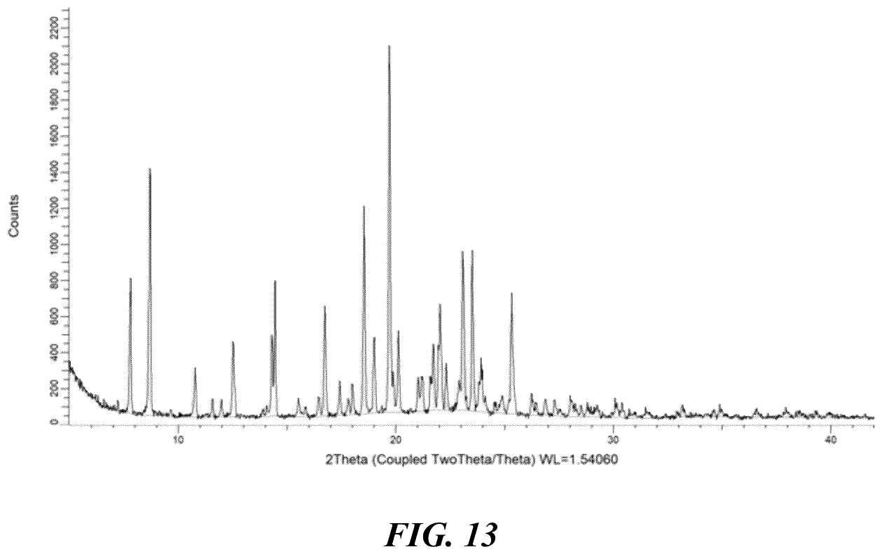

In embodiments, the liquid formulation comprises a succinate salt of Compound 1 and one or more pharmaceutically acceptable excipients. In embodiments, the succinate salt is succinate (1.5 equivalents) salt of Compound 1. In embodiments, succinate (1.5 equivalents) salt of Compound 1 is crystalline (see Example 15, Table 14, FIG. 13).

In embodiments, the liquid formulation comprises a tartrate salt of Compound 1 and one or more pharmaceutically acceptable excipients.

In embodiments, the liquid formulation comprises a L-tartrate salt of Compound 1 and one or more pharmaceutically acceptable excipients. In embodiments, the salt is monotartrate salt of Compound 1.

In embodiments, the liquid formulation comprises a pyruvate salt of Compound 1 and one or more pharmaceutically acceptable excipients.

In embodiments, the liquid formulation comprises a malate salt of Compound 1 and one or more pharmaceutically acceptable excipients.

In embodiments, the liquid formulation comprises a deuterated form of Compound 1 and one or more pharmaceutically acceptable excipients.

The pharmaceutically acceptable excipient of the liquid formulation can include a pH buffered aqueous solution, and optionally a pH modifier and/or a tonicity agent.

In embodiments, the pharmaceutically acceptable excipient of the liquid formulation comprises a pH buffered aqueous solution.

The pharmaceutically acceptable excipient of the liquid formulation comprises water. The water can act as a diluent and include, without limitation, water for injection (WFI), sterile water, bacteriostatic water for injection (BWFI), distilled water, bidistilled water, deionized water, deionized distilled water, and reverse osmosis water.

In embodiments, the formulation includes a buffer suitable for parenteral application, such as a phosphate or histidine buffer. The buffer is present in the formulation in an amount of about less than 100 mM. In embodiments a 50 mM or 10 mM phosphate buffer is used. In embodiments a 20 mM histidine buffer is used. Buffer composition and ionic strength of the buffer impacts osmolarity.

In embodiments, the formulation comprises a pH buffered aqueous solution. In embodiments, the pH buffered aqueous solution is a phosphate buffer solution or a histidine buffer solution. In embodiments, the pH buffered aqueous solution is a phosphate buffer solution. The phosphate buffer can contain monosodium phosphate, monopotassium phosphate, disodium phosphate, sodium tripolyphosphate, and the like. In embodiments, the phosphate buffer comprises disodium phosphate and monopotassium phosphate. In embodiments, the phosphate buffer comprises disodium phosphate, monopotassium phosphate, and one or more additional salts such as sodium chloride, potassium chloride, calcium chloride, and magnesium chloride. In embodiments, the phosphate buffer comprises disodium phosphate (i.e., sodium phosphate dibasic) and monosodium phosphate (i.e., sodium phosphate monobasic). In embodiments, the phosphate buffer comprises monopotassium phosphate and optionally sodium hydroxide.

In embodiments, the pH buffered aqueous solution is a phosphate buffer solution having a phosphate concentration of about 0.1 mM to about 500 mM, about 1 mM to about 400 mM, about 2 mM to about 250 mM, about 4 mM to about 100 mM, about 5 mM to about 50 mM, or about 10 mM to about 25 mM. In embodiments, the pH buffered aqueous solution is a phosphate buffer solution having a pH ranging from about 5 to about 8.5, from about 5.5 to about 8, from about 6 to about 7.5, or from about 6.5 to about 7.

In embodiments, the pH buffered aqueous solution is a phosphate buffer solution having a phosphate concentration of about 1 mM to about 100 mM, about 8 mM to about 40 mM, or about 10 mM to about 15 mM and a pH ranging from about 5.5 to about 6.5. In embodiments, the pH buffered aqueous solution is a phosphate buffer solution having a phosphate concentration of about 10 mM and a pH of about 6.

In embodiments, the pH buffered aqueous solution is a histidine buffer solution having a histidine concentration of about 0.1 mM to about 500 mM, about 1 mM to about 400 mM, about 2 mM to about 250 mM, about 4 mM to about 100 mM, about 5 mM to about 50 mM, or about 10 mM to about 25 mM. In embodiments, the pH buffered aqueous solution is a histidine buffer solution having a pH ranging from about 4 to about 8, from about 4.5 to about 7.5, from about 5 to about 7, or from about 5.5 to about 6.5, or about 6.

In embodiments, the pH buffered aqueous solution is a histidine buffer solution having a histidine concentration of about 1 mM to about 100 mM, about 10 mM to about 50 mM, or about 15 mM to about 25 mM and a pH ranging from about 5.5 to about 6.5. In embodiments, the pH buffered aqueous solution is a histidine buffer solution having a histidine concentration of about 20 mM and a pH of about 6.

In addition to phosphate and/or histidine buffer solutions, the liquid formulation disclosed herein can include other buffering agents. Non-limiting examples of additional buffering agents include phosphoric acid, citrate, succinate, gluconate, histidine, acetic acid, ascorbate, tartaric acid, maleic acid, glycine, lactate, lactic acid, ascorbic acid, imidazole, bicarbonate, carbonic acid, succinic acid, sodium benzoate, benzoic acid, gluconate, edetate, malate, imidazole, sodium acetate, Tris (i.e., tris (hydroxymethyl) aminomethane), and mixtures thereof.

In embodiments, the pharmaceutically acceptable excipient of the liquid formulation further comprises a pH modifier, which can be used to further adjust the pH of the formulation to a desired value. In embodiments, the pH modifier is sodium hydroxide. Non-limiting exemplary pH modifiers include sodium hydroxide, potassium hydroxide, and hydrochloric acid.

In embodiments, the pharmaceutically acceptable excipient of the liquid formulation further comprises a tonicity agent. The formulation may include tonicity agents to provide an osmolarity suitable for parenteral applications. In embodiments, the tonicity agent is sodium chloride, potassium chloride, and/or a sugar alcohol.

In embodiments, the tonicity agent is a sugar alcohol. Non-limiting example sugar alcohols include mannitol, ethylene glycol, glycerol, erythritol, threitol, lactitol, cellulose, maltitol, sorbitol, xylitol, glycerol, dextrose, pentaerythritol, maltotriitol, maltotetraitol, polyether polyols, polyester polyols, acrylic polyols, or any stereoisomers or post translational modifications thereof.

In embodiments, the tonicity agent is sodium chloride, potassium chloride, mannitol, glycerol, or a mixture thereof. In embodiments, the tonicity agent is sodium chloride, and/or mannitol.

In embodiments, the tonicity agent is sodium chloride. In embodiments, sodium chloride is present in the liquid formulation in a concentration of about 0.01 wt % to about 20 wt %, about 0.05 wt % to about 15 wt %, about 0.1 wt % to about 10 wt %, about 0.2 wt % to about 5 wt %, about 0.3 wt % to about 4 wt %, about 0.4 wt % to about 3 wt %, about 0.5 wt % to about 2 wt %, about 0.6 wt % to about 1 wt %, or about 0.7 wt % to about 0.8 wt %.

In embodiments, sodium chloride is present in the liquid formulation in a concentration of about 0.5 wt %. In embodiments, sodium chloride is present in the liquid formulation in a concentration of about 0.65 wt %. In embodiments, sodium chloride is present in the liquid formulation in a concentration of about 0.8 wt %.

Pharmaceutical formulations suitable for subcutaneous injection can have a pH of about 5.8-8.5. In embodiments, the liquid formulation disclosed herein has a pH ranging from about 5 to about 9.5, from about 5.5 to about 9, from about 5.8 to about 8.5, or from about 6.5 to about 7. In embodiments, the liquid formulation has a pH ranging from 6 to 7.6, from 6.2 to 7.4, from 6.4 to 7.2, from 6.6 to 7.0, or about 6.8. In embodiments, the pH of the liquid formulation may be ranging from about 6.0 to about 6.3.

In embodiments, the liquid formulation has a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In embodiments, the liquid formulation has a pH of 6.0±0.2, 6.5±0.2, or 7.4±0.2. In embodiments, the formulation has a pH of 6.0±0.1, 6.5±0.1, or 7.4±0.1.

In embodiments, the liquid formulation disclosed herein is acidic. In embodiments, the liquid formulation has a pH of 6.0±0.2 or 6.5±0.2. In embodiments, the formulation has a pH of 6.0±0.2.

The pH values disclosed herein can be measured using methods known to a person of ordinary skill in the art, e.g., on a Mettler Toledo FiveEasy pH meter calibrated with a 2-point calibration-pH 4.01 and 7.00 prior to analysis.

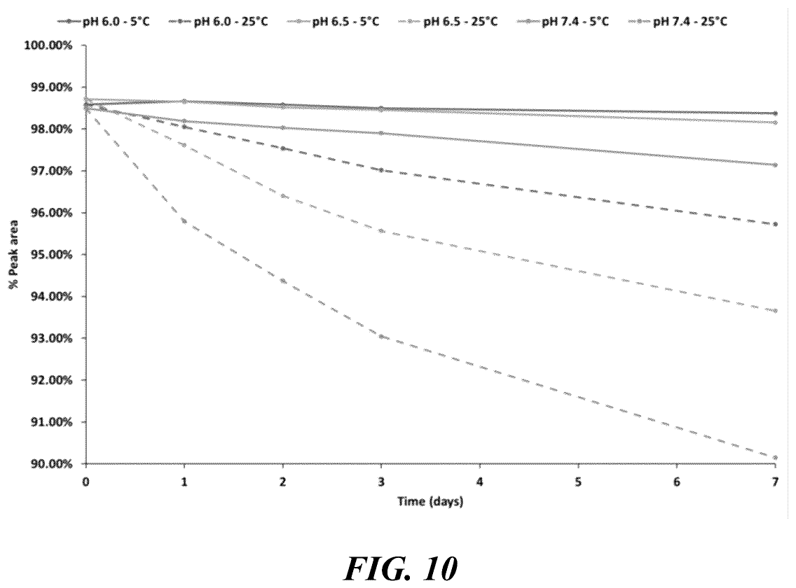

Surprisingly, it has been found that a mildly acidic pH (e.g., pH 6.0) yields a more homogeneous and stable formulation. For example, Compound 1 or its salt forms dissolves poorly at a pH below 5, while complete dissolution of up to 30 mg/ml of Compound 1 is observed when pH is above 5 (see Example 8). Also, as shown in FIG. 10 of Example 11, greater chemical stability of Compound 1 is achieved at mildly acidic conditions (e.g., pH 6.0) than at basic conditions (e.g., pH of 7.4). Specifically, a liquid formulation disclosed herein having a pH of about 7.4 degrades at least 4%, 5%, 6%, 7%, 8%, 10%, 12%, 15%, or 20% more than a substantially similar liquid formulation having a pH of about 6.0 when stored at 25° C. for 1-7 days, 2-6 days, or 3-5 days. As such, the mild acidity provides Compound 1 with enhanced stability and resistance to aggregation and degradation.

In embodiments, the liquid formulation has an osmolarity ranging from 200 mOsm/kg to 600 mOsm/kg. In embodiments, the liquid formulation has an osmolarity ranging from 200 mOsm/kg to 600 mOsm/kg, ranging from 225 mOsm/kg to 550 mOsm/kg, 250 mOsm/kg to 500 mOsm/kg, 275 mOsm/kg to 450 mOsm/kg, 300 mOsm/kg to 400 mOsm/kg, 325 mOsm/kg to 375 mOsm/kg, or about 350 mOsm/kg. In embodiments, the liquid formulation has an osmolarity ranging from about 280 mOsm/kg to about 400 mOsm/kg.

In embodiments, the liquid formulation has an osmolarity of about 250 mOsm/kg, about 260 mOsm/kg, about 270 mOsm/kg, about 280 mOsm/kg, about 290 mOsm/kg, about 300 mOsm/kg, about 310 mOsm/kg, about 320 mOsm/kg, about 330 mOsm/kg, about 340 mOsm/kg, about 350 mOsm/kg, or about 360 mOsm/kg. In embodiments, the formulation has an osmolarity of about 300 mOsm/kg.

The formulation disclosed herein in any of its embodiments can be formulated for parenteral administration. For therapeutic purposes, formulations for parenteral administration can be in the form of aqueous or non-aqueous isotonic sterile injection solutions or suspensions. The term “parenteral”, as used herein, includes subcutaneous, intravenous, intraperitoneal, intramuscular, and intralesional, or infusion techniques

In embodiments, the liquid formulation is formulated for parenteral administration.

In embodiments, the liquid formulation of is formulated for subcutaneous administration.

The active ingredient(s) (e.g., Compound 1 or the pharmaceutically acceptable salt thereof) can be dissolved or suspended in the aforementioned pharmaceutically acceptable excipient. The liquid formulation may comprise one or more additional pharmaceutically acceptable excipients, such as solvents, diluents, fillers, colorants, pigments, stabilizers, adjuvants, and/or antioxidants, as suited to the particular dosage form desired. Nonlimiting examples of an antioxidant that may be used include pyrroloquinoline quinone (PQQ), vitamin C, vitamin E (e.g., a tocopherol, such as a-tocopherol, p-tocopherol, or y-tocopherol, or a mixture thereof), p-carotene, a polyphenol (e.g., a phenolic acid, a stilbene, or a flavonoid), or an inorganic selenium. In some embodiments, the antioxidant is selected from the group consisting of pyrroloquinoline quinone (PQQ), vitamin C, vitamin E, p-carotene, a polyphenol, thioglycerol, sodium bisulfite, carnosine, N-acetylcarnosine, pyruvate, astaxanthin, glutathione, cysteine, cysteine and combinations thereof. The formulation may also include solubilizers and/or surfactants to increase solubility of the pharmaceutically acceptable salt form of Compound 1. Additional aqueous or non-aqueous excipients (e.g., solubilizers and/or surfactants) that may facilitate dissolution of the active ingredient include, but are not limited to, ethanol, benzyl alcohol, DMSO, polyethylene glycol, propylene glycol, corn oil, cottonseed oil, peanut oil, and sesame oil.

The formulations disclosed herein can also comprise a preservative agent. Exemplary preservative agents include, but are not limited to, ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, propyl gallate, phenol, m-cresol, benzyl alcohol, alpha-tocopherol, citric acid, ethylenediamine tetraacetic acid (EDTA), sorbitol, tartaric acid, phosphoric acid, benzalkonium chloride, phenoxyethanol, and methyl paraben.

In embodiments, the formulation disclosed herein is not formulated for oral administration. In embodiments, the formulation disclosed herein is not in the form of tablets, caplets, capsules, or pills.

In embodiments, the liquid formulation comprises: about 30 mg/mL to about 40 mg/ml of Compound 1, or an equivalent amount of a pharmaceutically acceptable salt thereof, relative to a total volume of the formulation, a phosphate buffer solution having a phosphate concentration of about 10 mM and a pH of about 6, sodium hydroxide, and about 0.1 wt % to about 5 wt % sodium chloride, wherein the formulation is formulated for parenteral administration (e.g., subcutaneous administration). In embodiments, the liquid formulation has a pH of 6.0±0.2, 6.5±0.2, or 7.4±0.2.

In embodiments, the liquid formulation comprises: about 30 mg/ml of Compound 1 (as a free base) relative to a total volume of the formulation, a phosphate buffer solution having a phosphate concentration of about 10 mM and a pH of about 6, sodium hydroxide, and about 0.65 wt % sodium chloride, wherein the formulation is formulated for parenteral administration (e.g., subcutaneous administration). In embodiments, the liquid formulation has a pH of 6.0±0.2, 6.5±0.2, or 7.4±0.2.

In embodiments, the liquid formulation comprises: about 40 mg/ml of Compound 1 (as a free base) relative to a total volume of the formulation, a phosphate buffer solution having a phosphate concentration of about 10 mM and a pH of about 6, sodium hydroxide, and about 0.5 wt % sodium chloride, wherein the formulation is formulated for parenteral administration (e.g., subcutaneous administration). In embodiments, the liquid formulation has a pH of 6.0±0.2, 6.5±0.2, or 7.4±0.2.

In embodiments, the liquid formulation comprises: about 40 mg/ml of a sesquifumarate salt of Compound 1 relative to a total volume of the formulation (the concentration here is determined by the weight of the sesquifumarate salt of Compound 1), a phosphate buffer solution having a phosphate concentration of about 10 mM and a pH of about 6, sodium hydroxide, and about 0.65 wt % sodium chloride, wherein the formulation is formulated for parenteral administration (e.g., subcutaneous administration). In embodiments, the liquid formulation has a pH of 6.0±0.2, 6.5±0.2, or 7.4±0.2.

In embodiments, the liquid formulation comprises: about 53 mg/ml of a sesquifumarate salt of Compound 1 relative to a total volume of the formulation (the concentration here is determined by the weight of the sesquifumarate salt of Compound 1), a phosphate buffer solution having a phosphate concentration of about 10 mM and a pH of about 6, sodium hydroxide, and about 0.5 wt % sodium chloride, wherein the formulation is formulated for parenteral administration (e.g., subcutaneous administration). In embodiments, the liquid formulation has a pH of 6.0±0.2, 6.5±0.2, or 7.4±0.2.

In embodiments, the liquid formulation of the present disclosure has a shelf-life of at least about 6 hours, about 12 hours, about 18 hours, about 24 hours, about 30 hours, about 36 hours, about 42 hours, or about 48 hours, at a temperature of about 15° C., about 25° C., or about 40° C. In embodiments, the liquid formulation has a shelf-life of more than about 6 hours, about 12 hours, about 18 hours, about 24 hours, about 30 hours, about 36 hours, about 42 hours, or about 48 hours, at a temperature of about 15° C., about 25° C., or about 40° C.

In embodiments, the liquid formulation has a shelf-life of at least 12 hours, 24 hours, or 48 hours at 25° C.

In embodiments, the liquid formulation has a shelf-life of at least 1 day, 2 days, 3 days, 4 days, 5 days, 6 days, 7 days, 8 days, 9 days, 10 days, 11 days, 12 days, 13 days, 14 days, 21 days, 28 days, 30 days, 2 months, 3 months, 6 months, or about 1 year, at a temperature of about −4° C., about 0° C., about 5° C., or about 10° C.

In embodiments, the formulation has a shelf-life of at least 4 days, 7 days, 14 days, 30 days, 2 months, or 3 months at 5° C.

The liquid formulation disclosed herein is suitable for long-term storage. In embodiments, the liquid formulation can be stored for more than 1 month, more than 3 months, more than 6 months, more than 12 months, or more than 24 months as a liquid at a temperature of about 2° C. to about 8° C., or frozen at a temperature of e.g., −20° C.

### Methods

In embodiments, the present disclosure provides a method of delivering a formulation comprising Compound 1 or a pharmaceutically acceptable salt thereof to a subject. The present disclosure also provides a method of delivering the liquid formulation described herein in any of its embodiments to a subject.

In embodiments, the present disclosure provides methods of treating a neuropsychiatric disease. In embodiments, the neuropsychiatric disorder is selected from anxiety disorder, attention deficit hyperactivity disorder (ADHD), depression (including treatment resistant depression), cluster headache, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, attention deficit syndrome, posttraumatic stress disorder (PTSD), premenstrual dysphoric disorder (PMDD), and premenstrual syndrome (PMS). In embodiments, the neuropsychiatric disorder is major depressive disorder.

In embodiments, the present disclosure provides methods of treating major depressive disorder.

In embodiments, the present disclosure provides methods of treating a serotonin 5-HT2A receptor associated disease/disorder. In embodiments, the 5-HT2A receptor associated disease or disorder is depression. In embodiments, the depression is treatment resistant depression.

In embodiments, the 5-HT2A receptor associated disease or disorder is an eating disorder. In embodiments, the eating disorder is anorexia nervosa.

In embodiments, the 5-HT2A receptor associated disease or disorder is an anxiety disorder.

In embodiments, the 5-HT2A receptor associated disease or disorder is bipolar I disorder.

In embodiments, the 5-HT2A receptor associated disease or disorder is bipolar II disorder.

In embodiments, the 5-HT2A receptor associated disease or disorder is major depressive disorder.

In embodiments, the 5-HT2A receptor associated disease or disorder is posttraumatic stress disorder (PTSD).

The methods disclosed herein in any aspects include administering the liquid formulation disclosed herein to the subject parenterally. In embodiments, the liquid formulation is administered to the subject subcutaneously.

In embodiments, the liquid formulation is administered subcutaneously in a volume ranging from about 0.05 mL to about 10 mL, from about 0.1 mL to about 7.5 mL, from about 0.2 mL to about 5 mL, from about 0.3 mL to about 4 mL, from about 0.4 mL to about 3 mL, from about 0.5 mL to about 2 mL, from about 0.6 mL to about 1.5 mL, from about 0.7 mL to about 1.2 mL, or from about 0.8 mL to about 1 mL.

In embodiments, the liquid formulation is administered subcutaneously in a volume ranging from about 0.1 mL to about 2 mL, from about 0.2 mL to about 1 mL, from about 0.25 mL to about 0.8 mL, or about 0.5 mL.

In embodiments, Compound 1 or a pharmaceutically acceptable salt thereof is administered subcutaneously at least once daily in an amount of about 0.01 mg to about 500 mg, about 0.1 mg to about 250 mg, about 1 mg to about 100 mg, about 2 mg to about 75 mg, about 4 mg to about 50 mg, about 5 mg to about 40 mg, about 10 mg to about 30 mg, or about 15 mg to about 20 mg.

In embodiments, the subject is a mammal. In embodiments, the subject is a human. In embodiments, the subject is an adult.

Unexpectedly, it has been discovered that adverse injection site reactions (e.g., pain) are prevented or reduced by the combination of components as excipients (i.e., pH buffered aqueous solution, pH modifier, and tonicity agent) in the liquid formulation for subcutaneous administration.

In embodiments, the method disclosed herein in any of its embodiments (e.g., subcutaneous administration of the liquid formulation) prevents or substantially eliminates injection site rection. In embodiments, the injection site reaction presents as pain, discoloration, redness, bruising, swelling, and/or scabbing.

In embodiments, the present disclosure provides methods of making a liquid formulation for parenteral administration to a subject. The method comprises: combining an effective amount of a pharmaceutically acceptable salt of Compound 1;

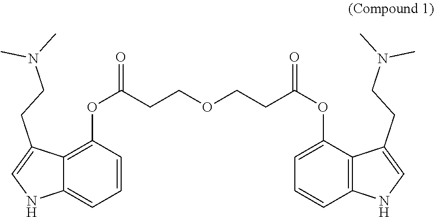

a pH buffered aqueous solution, and optionally a pharmaceutically acceptable excipient, thereby forming the liquid formulation. In embodiments, the method comprises combining an effective amount of the pharmaceutically acceptable salt of Compound 1, the pH buffered aqueous solution, and the pharmaceutically acceptable excipient. In embodiments, the formulation prepared by the method disclosed herein is formulated for subcutaneous administration.

The method may be used to prepare the aforementioned liquid formulation in any of its embodiments (e.g., having the composition, pH, osmolarity, and/or shelf-life as described herein). In embodiments, the salt of Compound 1, the pharmaceutically acceptable excipient, and the pH buffered aqueous solution are mixed simultaneously, each at the aforementioned amounts, to form the liquid formulation. In embodiments, the salt of Compound 1 and the pH buffered aqueous solution are mixed to form a first mixture, which can be subsequently mixed with the pharmaceutically acceptable excipient, if needed, to form the liquid formulation. In embodiments, the salt of Compound 1 and the pharmaceutically acceptable excipient if present are mixed to form a second mixture, which is subsequently mixed with the pH buffered aqueous solution to form the liquid formulation.

The mixing (i.e., combining) may be performed by agitation using e.g., an agitator, a vortexer (vortex mixer), a rotary shaker, a magnetic stirrer, a centrifugal mixer, an overhead stirrer, or a sonicator. In embodiments, the mixing is performed using a vortexer e.g., at a speed of 100-3,000 rpm, 200-2,000 rpm, or 500-1,000 rpm at a temperature of 0-40° C., 4-30° C., or 15-25° C. for 1 min to 2 hours, 5 min to 1 hour, or 15 min to 30 min, or until a homogeneous formulation is obtained.

The salt of Compound 1 used in the method may be the salt(s) disclosed herein in any of its embodiments. In embodiments, the salt of Compound 1 is crystalline. In embodiments, the salt is a sesquifumarate salt of Compound 1. In embodiments, the sesquifumarate salt of Compound 1 is crystalline, and is characterized by an XRPD pattern having peaks at 8.7±0.2, 19.7±0.2, and 23.6±0.2 °2θ. In embodiments, the sesquifumarate salt of Compound 1 is further characterized by peaks in an XRPD pattern at 14.4±0.2, 21.8±0.2, and 22.2±0.2 °2θ. In embodiments, the sesquifumarate salt of Compound 1 is characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °2θ.

The pH buffered aqueous solution used in the method may be the pH buffered aqueous solution(s) disclosed herein in any of its embodiments. In embodiments, the pH buffered aqueous solution used in the method comprises a phosphate buffer solution or a histidine buffer solution. In embodiments, the pH buffered aqueous solution is a phosphate buffer solution having a phosphate concentration of about 1 mM to about 100 mM and a pH ranging from about 5.5 to about 6.5. In embodiments, the pH buffered aqueous solution is a phosphate buffer solution having a phosphate concentration of about 10 mM and a pH of about 6. In embodiments, the phosphate buffer solution is a phosphate-buffered saline (PBS).

The pharmaceutically acceptable excipient used in the method may be the pharmaceutically acceptable excipient(s) disclosed herein in any of its embodiments.

In embodiments, the pharmaceutically acceptable excipient comprises a pH modifier. The pH modifier used in the method may be the pH modifier(s) disclosed herein in any of its embodiments. In embodiments, the pH modifier is sodium hydroxide.

In embodiments, the pharmaceutically acceptable excipient further comprises a tonicity agent. The tonicity agent used in the method may be the pH modifier(s) disclosed herein in any of its embodiments. In embodiments, the tonicity agent is sodium chloride or mannitol.

In embodiments, the liquid formulation prepared by the method disclosed herein has a pH ranging from about 5.8 to about 8.5. In embodiments, the formulation has a pH ranging from about 6 to about 7.5. In embodiments, the formulation has a pH of 6.0±0.2, 6.5±0.2, or 7.4±0.2. In embodiments, the formulation is acidic. In embodiments, the formulation has a pH of 6.0±0.2.

In embodiments, the formulation prepared by the method disclosed herein has an osmolarity ranging from 200 mOsm/kg to 600 mOsm/kg. In embodiments, the formulation has an osmolarity ranging from about 280 mOsm/kg to about 400 mOsm/kg. In embodiments, the formulation has an osmolarity of about 300 mOsm/kg.

### Kits

The components of the liquid formulation disclosed herein may be contained in at least two separate containers or compartments of a kit and mixed prior to administration, for example on the day of administration or immediately prior to administration. This can be beneficial because the salt of Compound 1 generally has better stability (e.g., longer shelf-life) in solid form than in solutions (see Examples).

In embodiments, the present disclosure provides a first container comprising a pharmaceutically acceptable salt of Compound 1

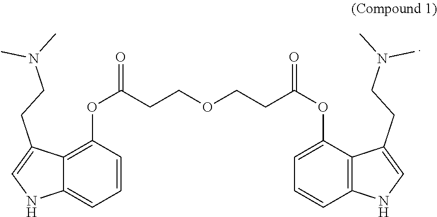

In embodiments, the first container comprises about 0.1 mg to 500 mg, about 0.5 mg to 250 mg, about 1 mg to 100 mg, about 5 mg to 75 mg, or about 10 mg to 50 mg of the salt of Compound 1.

The present disclosure also provides a kit comprising the first container disclosed herein in any embodiments and a second container comprising a pH buffered aqueous solution. In embodiments, the second container further comprises a pharmaceutically acceptable excipient.

The kit can include one or more containers for the liquid formulations as described herein. In some embodiments, the kit contains separate containers, dividers, or compartments for different components of the liquid formulation.

In embodiments, the kit has two or more containers, or divided into two or more portions, formulated to be shelf stable, and packaged separately. These separate portions are then mixed in appropriate amounts, for administration (e.g., subcutaneous injection). In embodiments, the kit includes a first container or portion which contains the solid portion (e.g., the salt of Compound 1), and a second container or portion which contains the liquid portion (e.g., pH buffered aqueous solution, pharmaceutically acceptable excipient). The separate components can be packaged in a re-sealable bottle, vial, syringe, package, cartridge, or capsule, which can be used, closed, and stored for multiple administrations if desired. In embodiments, the components of the kit can be packaged as one time use sealed bottle, vial, syringe, packages, cartridges, or capsules, e.g., in premeasured amounts, that can be mixed and used without measuring, and where the remaining one-time use packaging materials can then be discarded. The containers of the kits can be airtight, waterproof (e.g., impermeable to changes in moisture or evaporation), and/or light-tight.

The salt of Compound 1 present in the first container or the kit may be the salt(s) disclosed herein in any of its embodiments. In embodiments, the salt of Compound 1 present in the first container is in crystalline form. In embodiments, the salt is a sesquifumarate salt of Compound 1. In embodiments, the sesquifumarate salt of Compound 1 is crystalline, and is characterized by an XRPD pattern having peaks at 8.7±0.2, 19.7±0.2, and 23.6±0.2 °2θ. In embodiments, the sesquifumarate salt of Compound 1 is further characterized by peaks in an XRPD pattern at 14.4±0.2, 21.8±0.2, and 22.2±0.2 °2θ. In embodiments, the sesquifumarate salt of Compound 1 is characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °2θ.

The pH buffered aqueous solution of the kit may be the pH buffered aqueous solution(s) disclosed herein in any of its embodiments. In embodiments, the pH buffered aqueous solution present in the second container of the kit is a phosphate buffer solution having a phosphate concentration of about 1 mM to about 100 mM and a pH ranging from about 5.5 to about 6.5. In embodiments, the pH buffered aqueous solution is a phosphate-buffered saline (PBS).

The pharmaceutically acceptable excipient of the kit, if present, may be the pharmaceutically acceptable excipient(s) disclosed herein in any of its embodiments.

In embodiments, the pharmaceutically acceptable excipient present in the second container of the kit comprises a pH modifier. The pH modifier of the kit may be the pH modifier(s) disclosed herein in any of its embodiments. In embodiments, the pH modifier is sodium hydroxide.

In embodiments, the pharmaceutically acceptable excipient further comprises a tonicity agent. The tonicity agent of the kit may be the pH modifier(s) disclosed herein in any of its embodiments. In embodiments, the tonicity agent is sodium chloride or mannitol.

In embodiments, provided herein is a kit for delivering a formulation comprising a pharmaceutically acceptable salt of Compound 1 to a subject.

In embodiments, provided herein is a kit for treating a neuropsychiatric disease (e.g., as described herein).

In embodiments, provided herein is a kit for treating a serotonin 5-HT2A receptor associated disease/disorder (e.g., as described herein).

In embodiments of the kits provided herein, the kit comprises:
(i) one or more first containers each comprising about 1-10 mg of the salt of Compound 1 (e.g., sesquifumarate salt of Compound 1); and
(ii) one or more second containers each comprising a phosphate buffer solution having a phosphate concentration of about 10 mM and a pH of about 6, sodium hydroxide, and about 0.1 wt % to about 5 wt % sodium chloride.

In some embodiments, the kits of the present disclosure comprise directions for administration. For example, the kit can include instructions to administer the liquid formulation in a suitable manner to perform the methods described herein, e.g., in a suitable route of administration, dose, dosing intervals (e.g., as described herein).

From the foregoing, it will be appreciated that specific embodiments of the invention have been described herein for purposes of illustration, but that various modifications may be made without deviating from the scope of the invention. Accordingly, the invention is not limited except as by the appended claims.

### EXAMPLES

The present invention is further illustrated by reference to the following Examples. However, it is noted that that Examples, like the embodiments described above, are illustrative and are not to be construed as restricting the scope of the invention in any way.

### Example 1: Synthesis of Compound 1

The free base of Compound 1 was synthesized according to Scheme 1.

### Scheme 1: Synthesis of the Free Base of Compound 1

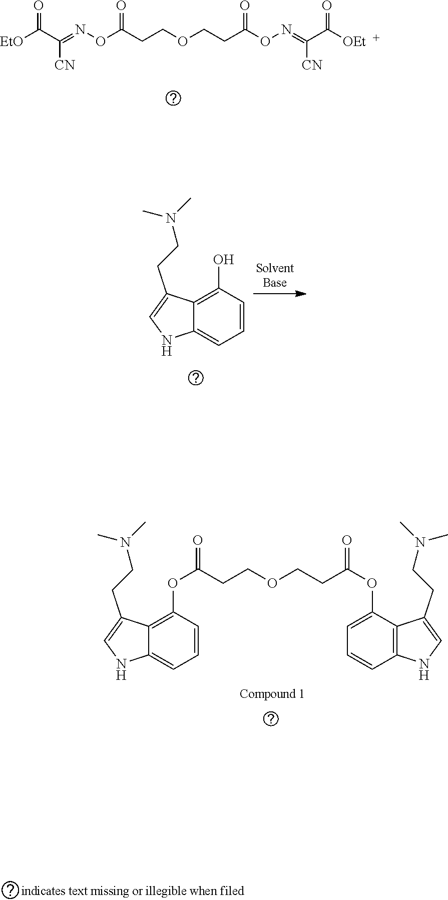

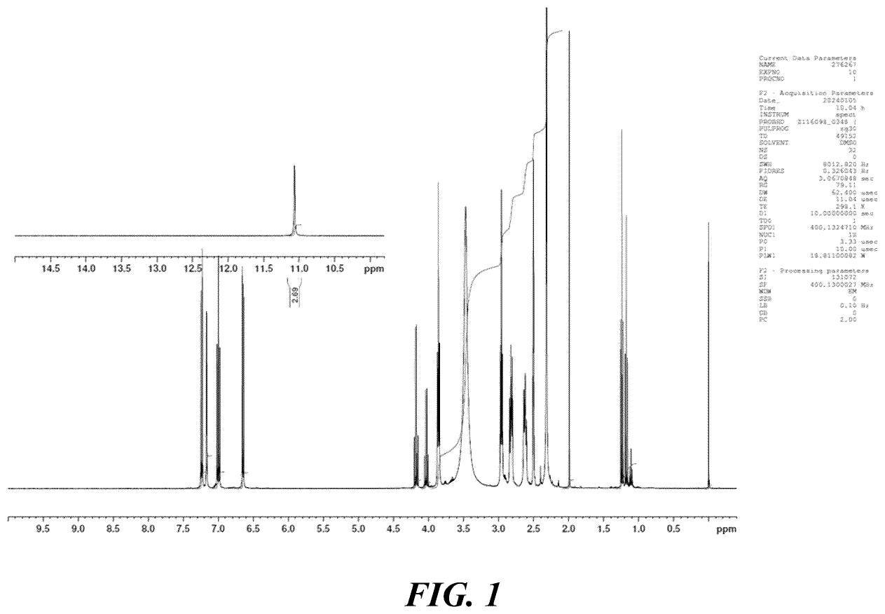

A 400 mL reactor vessel was charged with psilocin (35.51 g, 0.174 mol, circa 1.98 eq) and EtOAc (150 mL) and the resulting suspension stirred at 400 rpm under a nitrogen atmosphere with the reaction mixture held at 22° C. A solution of bis-oxyma linker (73% pure by qNMR analysis, 49.30 g, 87.7 mmol, 1.00 eq) in EtOAc (200 mL) was added in one portion and the resulting mixture stirred at 22° C. for 15 min, giving a red/brown suspension. The stirrer speed was increased to 600 rpm and stirring continued for a further 75 min. A second portion of psilocin (1.06 g, 5.19 mmol, circa 0.06 eq) was added in one portion and the resulting suspension stirred for 30 min. TEA (50 mL, 0.359 mol, 4.09 eq) was then added all at once and the reaction mixture stirred for 30 min until all solids had dissolved. The resulting orange/red solution was transferred to a separating funnel and the reaction vessel rinsed with EtOAc (175 mL). The resulting solution was washed sequentially with water (175 mL, measured to be at pH=9/10) and saturated brine (175 mL), then dried over magnesium sulfate, and filtered through filter paper with the filter cake being rinsed with EtOAc (50 mL). The resulting red/brown filtrate solution was concentrated in vacuo on a rotary evaporator with the water bath set at 31° C. with a max vacuum of 40 mbar to generate the free base of Compound 1 (40.27 g, 86%). 1H NMR is shown in FIG. 1. The free base of Compound 1 was a brown solid.

### Example 2: Synthesis of Bis-Formate Salt of Compound 1

### Characterization of Bis-Formate Salt of Compound 1:

A portion of the free base of Compound 1 was purified by chromatography using an Interchim Puriflash C18 column with a 5-95% acetonitrile in water linear gradient with 0.1 v/v % formic acid as a pH modifier. The relevant fractions were freeze-dried to give an amorphous bis-formate salt of Compound 1.

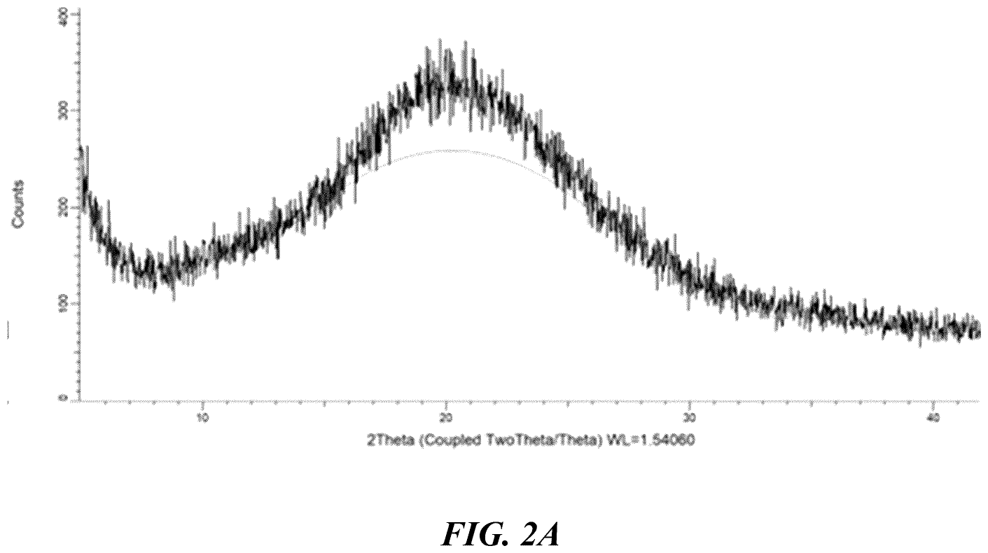

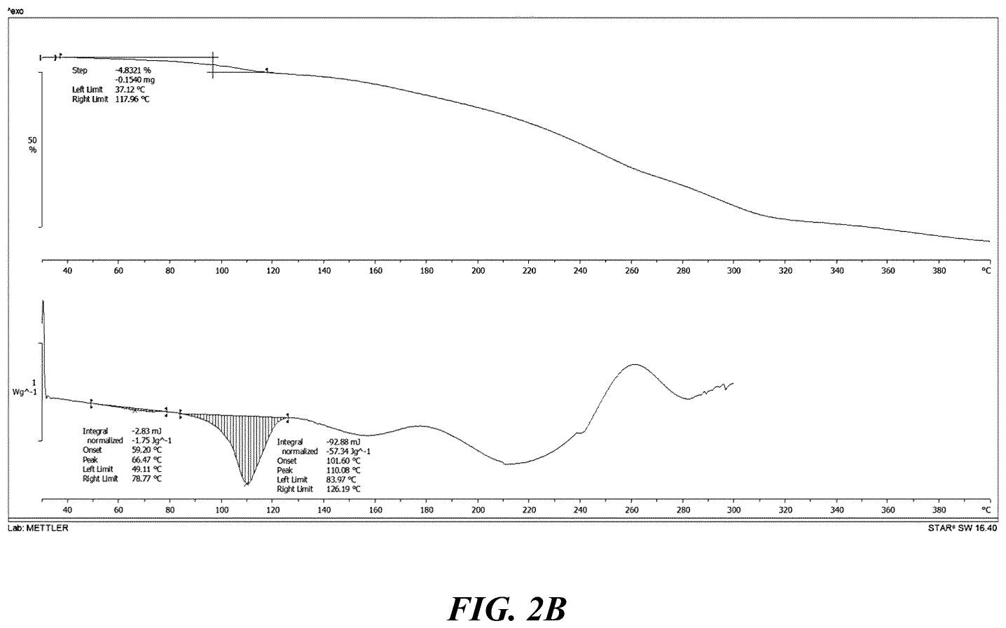

The bis-formate Salt of Compound 1 was obtained as an off white solid. It was characterized by XRPD and found to be amorphous (FIG. 2A). Thermal analysis by differential scanning calorimetry (DSC) showed endothermic events between 49-79° C., 84-126° C. and no evidence of recrystallisation, thermogravimetric Analysis (TGA) showed 4.8% mass loss between 30-118° C., followed by further mass loss (FIG. 2B).

### Solid State Stability of Bis-Formate Salt:

Portions of the bis-formate salt of Compound 1, with an initial UPLC purity of 99.5%, were placed in storage under the following conditions;
Storage at 40° C./75% humidity for 7 days
Storage at 25° C./96% humidity for 7 days
After 7 days both samples were analyzed by UPLC, and showed degradation.

After storage at 40° C./75% relative humidity for 7 days—UPLC purity 39%.

After storage at 25° C./96% relative humidity for 7 days—UPLC purity 80%.

### Solution Stability of Bis-Formate Salt:

An initial investigation into the solution stability of bis-formate salt in PBS at pH 7.4 and 5.8 and in acetonitrile:water (1:1) at 2, 4, and 44 hours was carried out at room temperature. The results are summarized in Table 1.

TABLE 1
Solution Stability of Bis-Formate Salt of Compound 1

Purity, area %

| Media | T = O | T = 2 hr | T = 4 hr | T = 44 hr |
|---|---|---|---|---|
| Acetonitrile:water | 97.6% | 97.0% | 96.9% | 92.8% |
| PBS at pH 7.4 | 98.2% | 98.2% | 97.9% | 91.9% |
| PBS at pH 5.8 | 98.1% | 98.2% | 98.3% | 98.1% |

### Example 3: Synthesis of Fumarate (1.0 Equivalents) Salt of Compound 1

Initial preparation of the fumarate (1.0 equivalents) of Compound 1:

The bis-formate salt of Compound 1 (490 mg) was dissolved in a mixture of water and acetonitrile (1:1, 30 mL). A solution of fumaric acid (90.8 mg, 1 equivalent) in a mixture of water and acetonitrile (1:1, 25 mL) was added, followed by additional water (10 mL). The resultant clear solution was flash frozen in a dry ice/acetone bath and then lyophilized to give the monofumarate salt of Compound 1 (534.5 mg). To remove residual formic acid, the salt was redissolved in a mixture of water and acetonitrile (1:1, 20 mL) and further water (5 mL) was added. The solution was flash frozen in a dry ice/acetone bath and then lyophilized to give the monofumarate salt of Compound 1 (524.8 mg) as an amorphous beige solid. The ¹H NMR was consistent with 1 equivalent of fumaric acid (FIG. 3A). UPLC purity 98.7%.

### Alternative Preparation of Fumarate (1.0 Equivalents) Salt of Compound 1:

200.49 mg fumarate (1 equivalent) amorphous salt of Compound 1 was weighed into a 7 mL vial and treated with acetonitrile (4 mL). The vial was capped, and the lid sealed with Parafilm®. The vial was wrapped in foil. The mixture (solvent+gum) was shaken at room temperature overnight. After shaking at room temperature overnight microscopy showed crystalline material.

A small portion was removed and analyzed by XRPD. The material was broken up slightly to loosen the layer on the bottom of the vial and shaking at room temperature was continued for a further 2 hours. The solid was isolated by filtration and dried briefly by suction and in vacuo (30 minutes) to give an off-white solid (173.77 mg).

### Characterization of the Fumarate (1.0 Equivalents) Salt of Compound 1:

The fumarate (1.0 equivalents) salt of Compound 1 was characterized by 1H NMR, UPLC, XRPD, DSC, TGA and GVS.

Analysis by XRPD shows the material to be crystalline (FIG. 3B, Table 2).

TABLE 2
Diffraction Peaks for the Fumarate (1.0 equivalents)
Salt of Compound 1 (“Form 1”)

| Peak number | Peak Position °2θ | Relative Intensity |
|---|---|---|
| Peak 1 | 5.266 | 11.34% |
| Peak 2 | 7.225 | 24.13% |
| Peak 3 | 7.663 | 4.88% |
| Peak 4 | 8.921 | 15.20% |
| Peak 5 | 10.441 | 33.56% |
| Peak 6 | 11.328 | 3.44% |
| Peak 7 | 12.777 | 45.24% |
| Peak 8 | 13.718 | 2.41% |
| Peak 9 | 14.424 | 7.27% |
| Peak 10 | 14.674 | 10.87% |
| Peak 11 | 15.364 | 15.15% |
| Peak 12 | 16.468 | 8.66% |
| Peak 13 | 16.865 | 19.21% |
| Peak 14 | 17.345 | 8.17% |
| Peak 15 | 17.853 | 40.66% |
| Peak 16 | 18.722 | 100.00% |
| Peak 17 | 19.343 | 24.48% |
| Peak 18 | 19.768 | 9.10% |
| Peak 19 | 20.756 | 9.06% |
| Peak 20 | 21.024 | 6.36% |
| Peak 21 | 21.417 | 35.02% |
| Peak 22 | 21.825 | 15.97% |
| Peak 23 | 22.791 | 8.61% |
| Peak 24 | 23.213 | 84.94% |
| Peak 25 | 24.474 | 6.25% |
| Peak 26 | 24.832 | 13.77% |
| Peak 27 | 25.719 | 2.20% |
| Peak 28 | 26.383 | 12.23% |
| Peak 29 | 27.434 | 3.71% |
| Peak 30 | 28.59 | 1.57% |
| Peak 31 | 29.034 | 3.41% |
| Peak 32 | 29.478 | 2.22% |
| Peak 33 | 30.154 | 1.93% |
| Peak 34 | 30.618 | 2.15% |
| Peak 35 | 31.336 | 3.14% |
| Peak 36 | 32.314 | 1.15% |
| Peak 37 | 32.688 | 4.60% |

¹H NMR of the fumarate (1.0 equivalents) salt of Compound 1 is consistent with structure and shows the presence of ca. 1.0 equivalent of fumarate, 0.04 equivalents of formate, and acetonitrile (0.34 equivalents). UPLC purity 99.5%. Thermal analysis by DSC shows a complex thermal profile with unresolved endothermic events between 88-142° C., endothermic events between 144-155° C. and 156-174° C. and an exothermic event between 175-212° C. (likely decomposition) (FIG. 3C). TGA analysis shows a 4.8% mass loss between 36-180° C., followed by decomposition above ca. 180° C. (FIG. 3D).

A GVS experiment to study the behavior of the fumarate (1.0 equivalents) salt of Compound 1 under conditions of varying relative humidity was carried out and shows the sample to be hygroscopic with a 22% increase in mass over 0-90% relative humidity range over the second sorption cycle. A portion was removed and XRPD analysis of the material post GVS shows poorly crystalline material with a change in XRPD pattern (FIGS. 3E-3F). UPLC analysis shows a reduction in purity (to 81.7%) post GVS.

### Solid State Stability of the Fumarate (1.0 Equivalents) Salt of Compound 1:

Portions of the fumarate (1.0 equivalents) salt of Compound 1, with an initial UPLC purity of 99.3%, were placed on storage under the following conditions:
Storage at 40° C./75% relative humidity for 7 days
Storage at 25° C./96% relative humidity for 7 days
After 7 days both samples were analyzed by UPLC and both showed degradation, especially at elevated temperature.

After storage at 40° C./75% relative humidity for 7 days—UPLC purity 57%. Shows a change in XRPD pattern.

After storage at 25° C./96% relative humidity for 7 days—UPLC purity 86.1%

### Example 4: Synthesis of Sesquifumarate Salt (1.5 Equivalents of Fumarate) of Compound 1

The free base of Compound 1 (11.43 g, 21.4 mmol, 1.00 eq) was dissolved in a mixture of 2-propanol (60 mL) and water (15 mL) to give a dark orange solution. Fumaric acid (4.97 g, 42.8 mmol, 2.00 eq) was added and the resulting suspension stirred at room temperature under nitrogen for 18 h to give a red/brown suspension. The suspended solid was collected by filtration on a P3 sintered-glass funnel, and the filter cake rinsed with 2:1 2-propanol/water (20 mL) then suction dried under a stream of nitrogen for 1 h to give an off-white solid that was further dried in a vacuum oven at 35° C./6 mbar for 20 h then at 50° C./6 mbar for 3 days to give sesquifumarate salt of Compound 1 (11.33 g, 75%) as a crystalline off-white solid.

The sesquifumarate (1.5 equivalents) salt of Compound 1 was characterized by ¹H NMR, UPLC, XRPD, DSC, TGA and GVS.

Analysis by XRPD shows the material to be crystalline (FIG. 4A, Table 3).

TABLE 3
Diffraction Peaks for the Fumarate (1.5
equivalents) Salt of Compound 1

| Peak | Peak Position | Relative |
|---|---|---|
| number | °2θ | Intensity |
| Peak 1 | 7.854 | 18.1% |
| Peak 2 | 8.725 | 76.9% |
| Peak 3 | 9.559 | 3.5% |
| Peak 4 | 11.117 | 18.4% |
| Peak 5 | 11.623 | 7.2% |
| Peak 6 | 12.294 | 26.4% |
| Peak 7 | 13.975 | 4.8% |
| Peak 8 | 14.401 | 66.2% |
| Peak 9 | 15.612 | 24.7% |
| Peak 10 | 16.33 | 4.6% |
| Peak 11 | 16.828 | 24.5% |
| Peak 12 | 17.16 | 7.3% |
| Peak 13 | 17.399 | 18.1% |
| Peak 14 | 17.784 | 6.8% |
| Peak 15 | 18.024 | 6.1% |
| Peak 16 | 18.388 | 5.4% |
| Peak 17 | 18.711 | 24.5% |
| Peak 18 | 19.237 | 74.0% |
| Peak 19 | 19.728 | 100.0% |
| Peak 20 | 20.052 | 38.7% |
| Peak 21 | 20.84 | 18.2% |
| Peak 22 | 21.067 | 21.9% |
| Peak 23 | 21.366 | 10.7% |
| Peak 24 | 21.799 | 55.6% |
| Peak 25 | 22.188 | 60.2% |
| Peak 26 | 22.499 | 13.4% |
| Peak 27 | 22.852 | 12.6% |
| Peak 28 | 23.192 | 30.5% |
| Peak 29 | 23.558 | 85.6% |
| Peak 30 | 24.332 | 12.3% |
| Peak 31 | 24.603 | 20.3% |
| Peak 32 | 25.292 | 28.1% |
| Peak 33 | 26.387 | 5.9% |
| Peak 34 | 26.81 | 7.6% |
| Peak 35 | 27.374 | 3.0% |
| Peak 36 | 28.188 | 8.2% |
| Peak 37 | 29.002 | 14.2% |
| Peak 38 | 29.485 | 4.3% |
| Peak 39 | 29.718 | 6.8% |
| Peak 40 | 30.051 | 3.4% |
| Peak 41 | 30.361 | 5.7% |
| Peak 42 | 30.89 | 2.1% |
| Peak 43 | 31.63 | 7.0% |
| Peak 44 | 33.212 | 5.9% |
| Peak 45 | 34.035 | 2.5% |
| Peak 46 | 34.705 | 2.9% |
| Peak 47 | 34.872 | 3.0% |
| Peak 48 | 35.38 | 2.3% |
| Peak 49 | 38.446 | 3.9% |
| Peak 50 | 39.004 | 2.7% |
| Peak 51 | 39.891 | 1.5% |

¹H NMR of the fumarate (1.5 equivalents) salt of Compound 1 is consistent with structure and shows the presence of ca. 1.5 equivalents of fumarate (FIG. 4B). UPLC purity 99.5%. Thermal analysis by DSC shows an endothermic event of onset 168° C. (likely melt), and a broad exothermic event between 176-221° C. (likely decomposition) (FIG. 4C). TGA analysis shows a 2% mass loss between 37-150° C., followed by decomposition above ca. 180° C. (FIG. 4D).

A GVS experiment to study the behavior of the fumarate (1.5 equivalents) salt of Compound 1 under conditions of varying relative humidity was carried out and shows a reversible profile with a 1.8% increase in mass over 0-90% relative humidity range over the second sorption cycle (FIG. 4E). XRPD analysis of the material post GVS shows material with no change in XRPD pattern. UPLC analysis shows no loss in purity post GVS (FIG. 4F).

### Stability Studies of the Fumarate (1.5 Equivalents) Salt of Compound 1

Storage at −20° C.: The batch of the fumarate (1.5 equivalents) salt of Compound 1 was stored in the freezer at −20° C. Samples were removed and analyzed by XRPD and UPLC after 3, 13 and 17 weeks. No change was observed by XRPD after 17 weeks (FIG. 4G). UPLC results are shown in Table 4.

TABLE 4
UPLC Purity After Storage in Freezer (−20° C.)

| Time point | UPLC Purity Storage in freezer (−20° C.) |
|---|---|
| Input | 99.5% |
| 3 weeks | 99.5% |
| 13 weeks | 99.5% |
| 17 weeks | 99.3% |

Storage at 40° C./75% relative humidity and 25° C./96% relative humidity: Portions of the fumarate (1.5 equivalents) salt of Compound 1 were stored under stress conditions of 40° C./75% relative humidity and 25° C./96% relative humidity. Samples were removed after 1 week, 4 weeks, 8 weeks and 12 weeks and analyzed by XRPD and UPLC. No change was observed by XRPD after 12 weeks (FIG. 4H). UPLC results are shown in Table 5.

TABLE 5
UPLC Purity After Storage Under Stress Conditions

| | UPLC Purity | UPLC Purity |
|---|---|---|
| Time point | Storage at 40° C./75% RH | Storage at 25° C./96% RH |
| Input | 99.5% | 99.5% |
| 7 days | 99.4% | 99.4% |
| 4 weeks | 99.2% | 99.2% |
| 8 weeks | 99.2% | 99.4% |
| 12 weeks | 99.3% | 99.3% |

### Example 5: Synthesis of Succinate Salt of Compound 1

The free base of Compound 1 was dissolved in acetone (3 mL) and treated with succinic acid in acetone. The reaction was shaken at room temperature overnight. For the experiment with 2 equivalents of succinic acid, the supernatant was decanted, and the residual material was divided into two portions. One portion was titrated with TBME. The second portion was dissolved in water (25 mL) and then IPA (100 mL) was added, and an oil formed. This was treated with a small quantity of the sesquifumarate salt of Compound 1 with the aim of inducing crystallization. After standing at room temperature with occasional shaking for 3.5 hours the sample crystallized as viewed by microscopy. The solids were characterized by ¹H NMR, UPLC, XRPD, DSC,

¹H NMR shows succinate (1.68 equivs), trace IPA (0.037 equivs), UPLC purity 99.2%. XRPD analysis of shows the material to be crystalline (FIG. 5A, Table 6). DSC shows broad endothermic events between 92-129° C. (onset 99° C.) and above 167° C. (likely decomposition) (FIG. 5B). TGA shows 1.9% mass loss between 37-128° C. and decomposition above ca. 167° C. (FIG. 5C).

Similar results were obtained in THF, IPA, and IPA/water mixture.

TABLE 6
Diffraction Peaks for the Succinate Salt of Compound 1

| Peak | Peak position | Relative |
|---|---|---|
| number | °2θ | Intensity |
| Peak 1 | 7.822 | 26.4% |
| Peak 2 | 8.695 | 58.2% |
| Peak 3 | 9.715 | 1.5% |
| Peak 4 | 10.778 | 24.1% |
| Peak s | 11.616 | 7.9% |
| Peak 6 | 12.004 | 2.7% |
| Peak 7 | 12.531 | 26.1% |
| Peak 8 | 13.877 | 3.9% |
| Peak 9 | 14.332 | 39.1% |
| Peak 10 | 15.57 | 10.9% |
| Peak 11 | 15.869 | 3.3% |
| Peak 12 | 16.461 | 5.1% |
| Peak 13 | 16.763 | 39.2% |
| Peak 14 | 17.411 | 8.9% |
| Peak 15 | 17.965 | 6.5% |
| Peak 16 | 18.562 | 40.6% |
| Peak 17 | 19.033 | 31.9% |
| Peak 18 | 19.744 | 100.0% |
| Peak 19 | 20.172 | 31.2% |
| Peak 20 | 20.698 | 2.1% |
| Peak 21 | 21.245 | 15.7% |
| Peak 22 | 21.688 | 14.3% |
| Peak 23 | 22.07 | 67.9% |
| Peak 24 | 22.323 | 8.2% |
| Peak 25 | 23.135 | 69.8% |
| Peak 26 | 23.563 | 54.4% |
| Peak 27 | 23.855 | 23.4% |
| Peak 28 | 24.568 | 5.1% |
| Peak 29 | 24.929 | 5.4% |
| Peak 30 | 25.349 | 31.3% |
| Peak 31 | 26.23 | 3.9% |
| Peak 32 | 26.89 | 9.2% |
| Peak 33 | 27.555 | 4.8% |
| Peak 34 | 28.088 | 7.9% |
| Peak 35 | 28.293 | 9.2% |
| Peak 36 | 28.859 | 6.2% |
| Peak 37 | 29.312 | 9.6% |
| Peak 38 | 30.207 | 7.2% |
| Peak 39 | 30.427 | 6.7% |
| Peak 40 | 33.214 | 5.2% |
| Peak 41 | 34.615 | 4.6% |
| Peak 42 | 35.058 | 3.7% |
| Peak 43 | 35.65 | 2.1% |
| Peak 44 | 37.95 | 3.2% |
| Peak 45 | 38.653 | 1.5% |
| Peak 46 | 39.345 | 2.5% |

### Example 6: Synthesis of L-Tartrate Salt of Compound 1

The free base of Compound 1 was weighed into a vial, treated with IPA (125 μL), and briefly shaken to give solutions. L-tartaric acid as a solution in THF was then carefully added to the free base solution.

L-tartaric acid salt (1 equivalent) showed an amorphous solid by XRPD. 1H NMR consistent was consistent with the structure (shows tartrate ca. 1.0 equivalent, THF 0.067 equivalents, IPA 0.02 equivalents and small aromatic impurities). A portion of this material (2.0 mg) was treated with IPA (50 μL) and shaken at room temperature. After 1 week some crystalline material was visible by optical microscopy. XRPD analysis showed some weak diffraction peaks (FIG. 6A). Thermal analysis by DSC showed and endothermic event of onset 170° C. (FIG. 6B).

L-tartaric acid salt (2 equivalents) showed an amorphous solid by XRPD. 1H NMR was consistent with the structure (shows tartrate ca. 1.0 equivalent, THF 0.21 equivalents, IPA 0.57 equivalents and small aromatic impurities). A portion of this material (2.52 mg) was treated with IPA (50 μL) and shaken at room temperature. After 3 weeks some crystalline material was visible by optical microscopy. XRPD analysis (2 equivalents of L-tartaric acid) showed some weak diffraction peaks of the same pattern as (1 equivalents of L-tartaric acid).

### Example 7: Stability Analysis of Compound 1 (Free Base) at Different pH Values

One of the key attributes of the target product profile was chemical stability (minimum ≥12 hours), which would ensure there is a window after preparation, so any delays in dosing would not require repreparation. Previous characterization of Compound 1 identified rapid chemical degradation at pH 7.4 under ambient conditions, with both the monomer and psilocin identified as key degradants.

Therefore, the pH stability of Compound 1 (free base) was assessed in 50 mM phosphate buffer at pH 6.0, 6.6 and 7.0 under ambient conditions, protected from light, at a concentration of 15 mg/mL². Stability was assessed using UPLC-UV detection, with monitoring of the peak areas of Compound 1, the known degradants and any additional peaks detected. There was no evidence of precipitation during the study and pH was stable for the duration of the assessment in each sample. However, discoloration was observed at the 48-hour timepoint in the pH 6.6 and 7 samples, more significantly at PH 7.

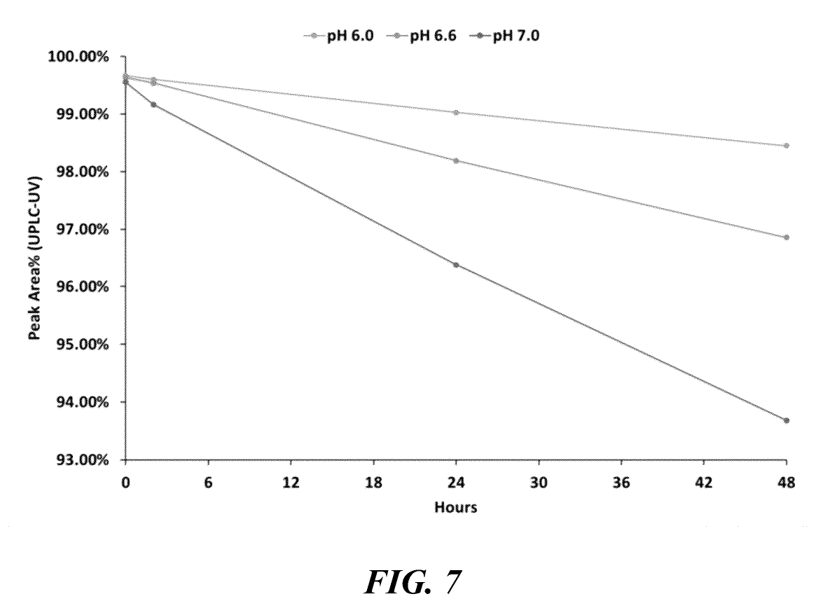

FIG. 7 (and Table 7) shows that chemical degradation occurred more rapidly at higher pH, and that in pH 6.0 there was ≤1% drop in chemical purity by peak area after 24 hours and <2% drop after 48 hours. Therefore, to ensure ≥24 hours chemical stability, the formulation was formulated at pH 6.0.

TABLE 7
Complete chromatography analysis of chemical degradation at pH 6.0, 6.6, and 7.0, respectively.

| Comp. | | | | 0 h | | | 2 h | | | 20 h | |
|---|---|---|---|---|---|---|---|---|---|---|---|
| 1 | m/z | RT | RRT | RT | area | % | RT | area | % | RT | area |
| pH 6.0 | 535 | 3.72 | 1.00 | 3.716 | 3053054 | 99.67% | 3.722 | 2554616 | 99.60% | 3.736 | 3005882 |
| pH 6.6 | 535 | 3.72 | 1.00 | 3.727 | 2278912 | 99.63% | 3.724 | 2245951 | 99.54% | 3.732 | 2620009 |
| pH 7.0 | 535 | 3.71 | 1.00 | 3.707 | 3565600 | 99.55% | 3.721 | 2362761 | 99.17% | 3.728 | 2947468 |

| Comp. | 20 h | 24 h | | | 42 h | | | 48 h | | |
|---|---|---|---|---|---|---|---|---|---|---|
| 1 | % | RT | area | % | RT | area | % | RT | area | % |
| pH 6.0 | 99.22% | 3.677 | 5790381 | 99.03% | 3.692 | 4753628 | 98.62% | 3.677 | 5436022 | 98.45% |
| pH 6.6 | 98.50% | 3.682 | 5418655 | 98.19% | 3.683 | 5621329 | 97.14% | 3.672 | 5493027 | 96.86% |
| pH 7.0 | 97.13% | 3.691 | 5253080 | 96.38% | 3.687 | 4781535 | 94.24% | 3.692 | 3988739 | 93.68% |

### Example 8: Solubility of Compound 1 Sesquifumarate in Buffer

The minimum target concentration of 15 mg/mL Compound 1 (free base) was easily attained during the stability assessment and there was scope to increase the concentration in the formulation to enable a reduced dose volume for preclinical studies, which was preferred.

Phosphate and histidine buffers both have buffering capacity across the subcutaneous pH range and have historical usage in parenteral application (Laursen, T., Hansen, B., Fisker, S., (2006) Pain perception after subcutaneous injections of media containing different buffers, Basic & Clinical Pharmacology & Toxicology, 98 (2), 218-221). Solubility was assessed in 50 mM phosphate buffer and 20 mM histidine buffer at pH 6.0, and visual solubility of 15 mg/mL was obtained with rapid dissolution in both buffer systems. As phosphate buffer is more widely used (Broadhead, J., Gibson, M., (2009) Parenteral dosage forms. In: Pharmaceutical preformulation and formulation. New York: Informa healthcare; 325-347) it was progressed for further evaluation.

On addition of additional Compound 1 sesquifumarate to increase concentration, incomplete dissolution was observed and a drop in pH was recorded (pH<5). It is known that fumaric acid has pH dependent solubility, with poor dissolution at pH<5 (Engel, C. A. R, ter Horst, J. H., Pieterse, M., van der Wielen, L. A. M., Straathof, A. J. J., (2013), Solubility of fumaric acid and its monosodium salt, Ind. Eng. Chem. Res., 52, 9454-9460); therefore, pH adjustment to pH>5 with NaOH was required to ensure dissolution of the fumaric acid counterion. This shows that the solubility of the pharmaceutically acceptable salt form of Compound 1 may also be increased by the addition of a pH modifier (e.g., NaOH), which increases the pH to >5.

Solubility of sesquifumarate salt of Compound 1 was assessed visually at 30 mg/mL Compound 1 (concentration determined as the amount of Compound 1 as free base), and on pH adjustment to >pH 5 rapid and complete dissolution was observed. To ensure stability was not concentration dependent, stability was assessed at ambient conditions, protected from light, from 22.5-30.0 mg/mL Compound 1 (free base) over 3 days.

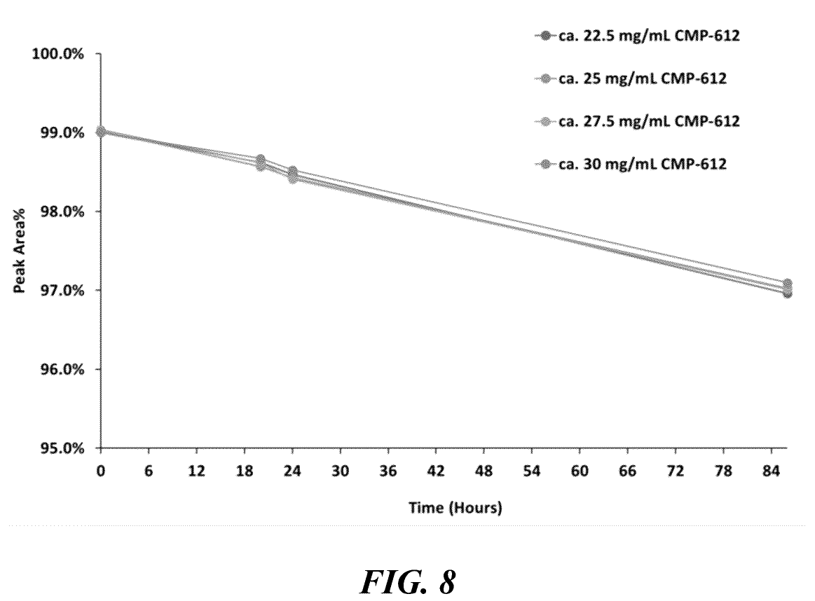

The pH remained stable for the study duration and there was no evidence of precipitation, however, discoloration was observed after 3 days in all samples. FIG. 8 shows at each concentration there was ca. 2% drop in purity by peak area after 3 days; this indicates that the chemical degradation was not concentration dependent. Increasing the formulation dose concentration to 30 mg/mL Compound 1 (free base) would allow a two-fold reduction in dose volume for preclinical studies.

The sesquifumarate salt of Compound 1 exhibited unexpectedly high solubility in aqueous solutions. Notably, the solubility of the bis-formate salt of Compound 1 in PBS at pH 7.4 was 3 mg/mL. By comparison, the solubility of the sesquifumarate salt of Compound 1 under the same conditions was >10 mg/mL.

### Example 9: pH Adjustment

Following the identification of a suitable buffer system and pH, the formulation required optimization for subcutaneous dosing with consideration to buffer concentration and tonicity to mitigate risk of pain at injection site and improve local site tolerance.

It is reported that a 50 mM phosphate buffer at pH 6.0 causes pain at the injection site. (Usach, I., Martinez, R., Festini, T., Peris, J. E., (2019) Subcutaneous injection of drugs: literature review of factors influencing pain sensation at the injection site, Adv. Ther., 36 (11), 2986-2996, and Fransson, J., Espander-Jansson, A., (1996) Local tolerance of subcutaneous injections, J. Pharm. Pharmacol., 48 (10), 1012-1015). Therefore, the buffer concentration was reduced to 10 mM phosphate in attempt to mitigate injection site pain. A visual assessment in the 10 mM phosphate buffer confirmed that the solubility was not impacted by buffer concentration.

Following this assessment an increased solution concentration of 40 mg/mL Compound 1 (free base) was assessed and determined feasible. However, it was noted that at this concentration dissolution was slower, requiring 15 minutes mixing on small scale (2 mL); although this meets the minimum criteria of 30 minutes, it is anticipated that this could extend beyond 30 minutes with increased volume. The rapid dissolution which was attained at 30 mg/mL was not obtained at 40 mg/mL Compound 1 (free base). Also, increased duration of contact between Compound 1 and aqueous media before dissolution appeared to increase the risk of chemical degradation by hydrolysis. Therefore, the solubility was not assessed beyond 40 mg/mL Compound 1 (free base), and it was selected as the highest concentration for formulation development.

Tonicity adjustment was assessed with NaCl and mannitol, two common tonicity agents used in parenteral applications, both successfully adjusted the tonicity to bring the osmolality to ca. 300 mOsm. It has been reported that mannitol can impact the blood-brain barrier (Chu, C., Jablonska, A., Gao, Y., Lan, X., Lesniak, W. G., Liang, Y., Liu, G., Li, S., Magnus, T., Pearl, M., Janowski, M., Walczak, P., (2022) Hyperosmolar blood-brain barrier opening using intra-arterial injection of hyperosmotic mannitol in mice under real-time MRI guidance, Nat. Protoc., 17 (1), 76-94), which may impact the therapeutic effect of Compound 1, therefore NaCl was preferred for tonicity adjustment, and the amount was optimized for both 30 and 40 mg/mL Compound 1 (free base) (Table 8).

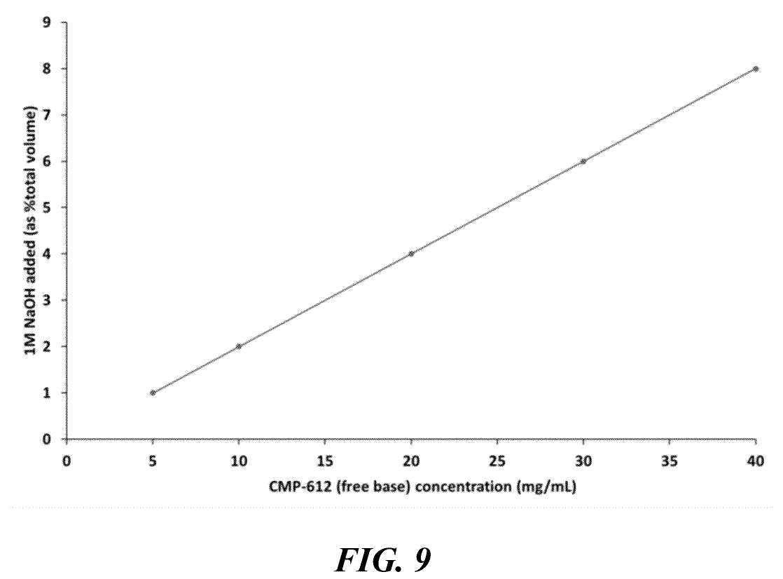

Also, for ease of formulation preparation the pH adjustment was simplified into a single step, with the identification of a linear relationship between Compound 1 (free base) concentration and required NaOH (1 M) addition (FIG. 9). In particular, FIG. 9 shows the addition of 1M of NaOH to adjust Compound 1 sesquifumarate formulation to pH 6.0 as a function of % total volume. In Table 8 below exemplary formulations are listed.

TABLE 8
Formulation details of Compound 1 sesquifumarate
at 30 and 40 mg/mL Compound 1 (free base)

| | | Amount | |
|---|---|---|---|
| | | 30 mg/mL | 40 mg/mL |
| Component | Function | Compound 1 | Compound 1 |
| Compound 1 (1.5 eq. | API | 39.9 mg/mL | 53.2 mg/mL |
| Fumarate salt)₃ | | (≡ 30 mg/mL | (≡ 40 mg/mL |
| | | Compound 1) | Compound 1) |
| 10 mM phosphate | Buffer | q.s. 100% | q.s. 100% |
| buffer (pH 6.0) | | | |
| NaCl | Tonicity | 0.65% wt | 0.50% wt |
| | agent | | |
| 1M NaOH | pH modifier | 6% total | 8% total |
| | | volume | volume |

To facilitate the planned preclinical studies, which have the potential of requiring different dose concentrations, the concentration of NaCl (tonicity agent) was modified to ensure the osmolality remained within a suitable dosing range across a likely dosing concentration range (2-30 mg/mL Compound 1 (free base)). A suitable concentration of NaCl was experimentally determined to be 0.8% wt as this enabled the osmolality across the concentration range to be 290-380 mOsm.

### Example 10: Filter Binding

Subcutaneous dosing requires sterilization prior to dosing, and the common method of sterilization in the preclinical setting is filtration through a 0.22 μm pore, however there is a risk of the drug compound binding to the filter membrane. Therefore, filter binding (n=1) was assessed using three common membranes (PTFE, PES and PVDF) and the results in Table 9 show there was no significant risk of filter binding as the peak areas post filtration were consistent with the unfiltered formulation.

TABLE 9
Filter binding assessment reported as
% peak area of unfiltered formulation

| Filter membrane (0.22 μm) | Peak Area | % unfiltered peak area |
|---|---|---|
| Unfiltered | 17873266 | 100.0% |
| PTFE | 17767626 | 99.4% |
| PES | 17819544 | 99.7% |
| PVDF | 17837824 | 99.8% |

A salt correction factor of 1.33 was used for the formulation development.

### Example 11: Stability Analysis

Formulation stability was assessed at the highest concentration (40 mg/ml COMPOUND 1 (free base) as per Table 8) across pH's 6.0, 6.5 and 7.4 with storage at 5 and 25° C. for a week. Stability was assessed for: Visual appearance for any precipitation or discoloration, UPLC for purity assessment by peak area %, pH.

There was no evidence of precipitation under any of the conditions tested, and FIG. 10 shows comparable results to the pH stability assessment previously carried out in Example 7, the chemical stability was better at lower pH. Additionally, the study showed that chemical stability was significantly improved at 5° C. with no drop in purity and no discoloration observed, whilst at 25° C. all samples showed discoloration after 2 days.

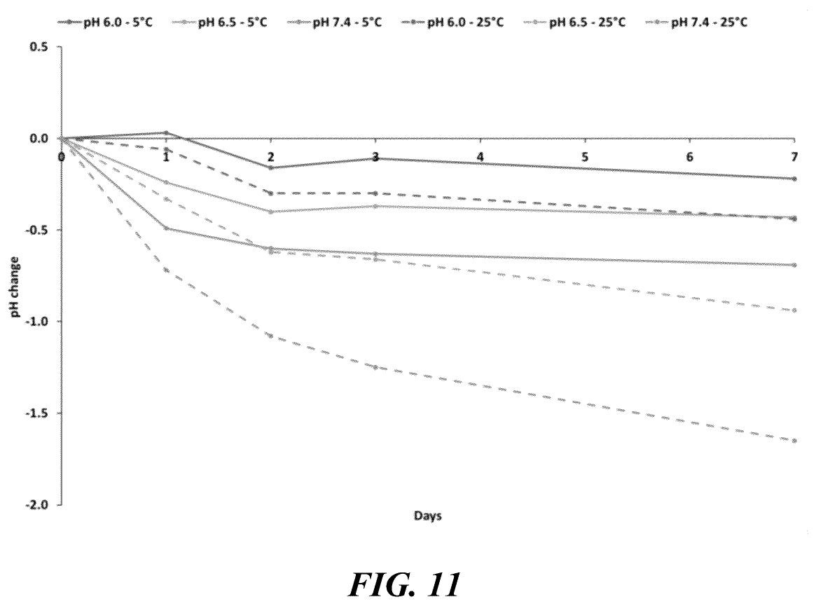

However, it was noted that due to the lower buffer capacity of 10 mM phosphate—compared to the previously tested 50 mM phosphate—pH changes were observed on storage (FIG. 11). The pH change became more significant at higher pH levels and at higher temperature (e.g., 25° C.).

The stability study therefore corroborates the earlier stability data of improved stability at pH 6.0 and shows that refrigerated storage can be used to further extend formulation shelf-life. The complete chromatography peak analyses demonstrating the formulation stability at pH 6.0, 6.5 and 7.4 at 5° C. and 25° C. over a 7-day period are shown in Tables 10-12.

TABLE 10
Summary Table Formulation Stability (pH 6.0) at 5° C. and 25° C. storage

| | | | 0 d | | | 1 d | | | 2 d |
|---|---|---|---|---|---|---|---|---|---|
| Comp. 1 | m/z | RRT | RT | area | % | RT | area | % | RT |
| pH 6.0 - 5° C. | 535 | 3.69 | 3.67 | 5803163 | 98.58% | 3.68 | 5346804 | 98.67% | 3.70 |
| pH 6.0 - 25° C. | 535 | 3.67 | 3.67 | 5803163 | 98.58% | 3.68 | 6058526 | 98.05% | 3.665 |

| | 2 d | | 3 d | | | 7 d | | |
|---|---|---|---|---|---|---|---|---|
| Comp. 1 | area | % | RT | area | % | RT | area | % |
| pH 6.0 - 5° C. | 3510523 | 98.58% | 3.689 | 4016808 | 98.50% | 3.702 | 5152071 | 98.37% |
| pH 6.0 - 25° C. | 6111488 | 97.54% | 3.659 | 5826362 | 97.02% | 3.706 | 4041209 | 95.72% |

TABLE 11
Summary Table Formulation Stability (pH 6.5) at 5° C. and 25° C. storage

| | | | 0 d | | | 1 d | | | 2 d |
|---|---|---|---|---|---|---|---|---|---|
| Comp. 1 | m/z | RRT | RT | area | % | RT | area | % | RT |
| pH 6.5 - 5° C. | 535 | 3.68 | 3.66 | 6000206 | 98.71% | 3.67 | 6364072 | 98.65% | 3.68 |
| pH 6.5 - 25° C. | 535 | 3.67 | 3.66 | 6000206 | 98.71% | 3.67 | 6221818 | 97.61% | 3.67 |

| | 2 d | | 3 d | | | 7 d | | |
|---|---|---|---|---|---|---|---|---|
| Comp. 1 | area | % | RT | area | % | RT | area | % |
| pH 6.5 - 5° C. | 5305404 | 98.52% | 3.69 | 3709762 | 98.45% | 3.71 | 4405813 | 98.15% |
| pH 6.5 - 25° C. | 6100230 | 96.41% | 3.66 | 5821558 | 95.57% | 3.70 | 4571646 | 93.66% |

TABLE 12
Summary Table Formulation Stability (pH 7.4) at 5° C. and 25° C. storage

| | | | 0 d | | | 1 d | | | 2 d |
|---|---|---|---|---|---|---|---|---|---|
| Comp. 1 | m/z | RRT | RT | area | % | RT | area | % | RT |
| pH 7.4 - 5° C. | 535 | 3.68 | 3.64 | 7658515 | 98.50% | 3.70 | 4882526 | 98.19% | 3.68 |
| pH 7.4 - 25° C. | 535 | 3.68 | 3.64 | 7658515 | 98.50% | 3.68 | 5942824 | 95.79% | 3.69 |

| | 2 d | | 3 d | | | 7 d | | |
|---|---|---|---|---|---|---|---|---|
| Comp. 1 | area | % | RT | area | % | RT | area | % |
| pH 7.4 - 5° C. | 4977671 | 98.03% | 3.69 | 4085888 | 97.90% | 3.69 | 5137777 | 97.14% |
| pH 7.4 - 25° C. | 4226459 | 94.37% | 3.68 | 4359959 | 93.04% | 3.71 | 3991121 | 90.15% |

### Example 12: pH Adjustment, Single Addition, and Incremental Addition

It was noted in Example 7 that the initial (t=0) samples had a lower purity (peak area %) than reported for the Compound 1 fumarate salt post-manufacture. This was potentially due to: degradation on storage of the material, method of pH adjustment, increased concentration of Compound 1 (free base).

Stability on storage at −20° C. was established for 3 months, therefore the drop observed was not due to compound degradation on storage.

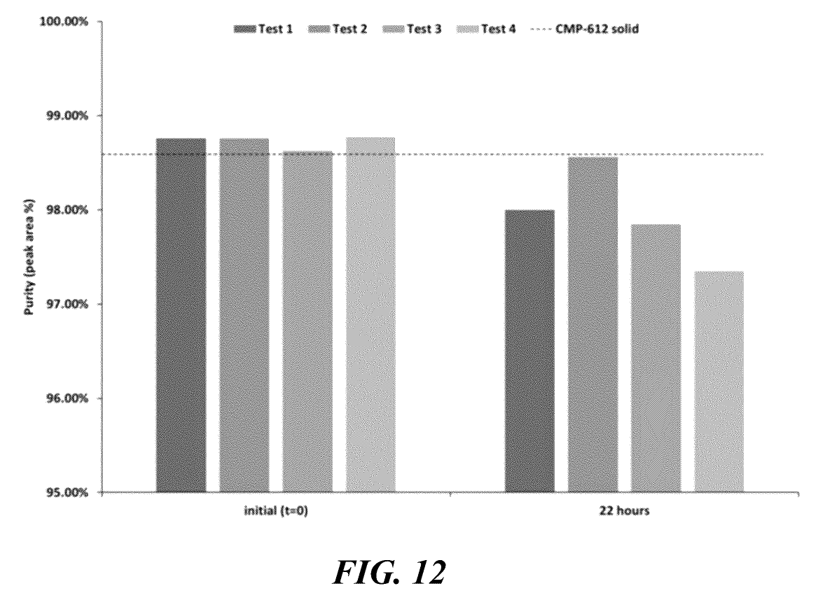

An evaluation was carried out varying pH adjustment to assess if this was leading to chemical degradation; and to also evaluate if the observations were concentration dependent the assessment was carried out at 30 mg/mL Compound 1 (free base) to replicate prior stability assessments. The pH addition was assessed as a single addition of NaOH and in incremental addition (3 equal increments) using both 1 M and 0.2 M NaOH. The tests were performed as follows and results are summarized in FIG. 12:
Test 1: single step addition of 1 M NaOH
Test 2: incremental addition of 1 M NaOH
Test 3: single step addition of 0.2 M NaOH
Test 4: incremental addition of 0.2 M NaOH

It was noted that the samples pH adjusted with 0.2 M NaOH had a slower dissolution and required additional mixing to obtain a solution. FIG. 12 shows that no drop in purity (% peak area) compared to compound purity was observed in any of the experiments at t=0, therefore, pH adjustment with NaOH did not impact initial stability.

On re-evaluation after 22 hours Test 2 showed a comparable degradation profile to previous evaluations; however, the 0.2 M NaOH Tests showed a higher degree of chemical degradation than both 1 M NaOH Tests.

Both the 0.2 M NaOH and 40 mg/ml Compound 1 (free base) formulations had a slower dissolution and showed more significant chemical degradation; this concludes that dissolution time (time for a solution to be obtained) impacts chemical degradation, and anything but rapid dissolution runs the risk of chemical degradation. To ensure minimal risk of chemical degradation the addition of NaOH (1 M) incrementally was recommended.

### Example 13: Formulation Profile

In some embodiments the pharmaceutical formulation has a profile as summarized in Table 13 below.

TABLE 13
Exemplary formulation profiles

| Target Product | | | | | | |
|---|---|---|---|---|---|---|
| Profile | | Target | | Ideal | | Formulation |
| Overall | Irritation at | No severe | | None | | |
| | administration | irritation | | | | |
| | site | | | | | |
| | Dosing volume | 1 | mL | 0.25-0.80 | mL | |
| Compound 1 | Solution | 12 | hours | 48 | hours | 24 hours |
| | stability | | | | | (RT) |
| | at RT | | | | | 7 days |
| | | | | | | (5° C.) |
| | Solubility | 15 | mg/mL | 40 | mg/mL | >30 mg/mL |
| | Dissolution | <20 | minutes | Immediate | | Immediate on |
| | rate | | | | | pH adjustment |
| | | | | | | (S 30 mg/mL) |
| Formulation | Osmolarity | 200-600 | | 300 | | 280-400 |
| | (mOsm/kg) | | | | | |
| | Buffering | <100 | mM | — | | 10 mM |
| | capacity | | | | | phosphate |
| | pH | 5.8-8.5 | | 7.4 | | 6.0-6.3 |
| | Excipients, | | | None | | NaCl |
| | preservatives | | | | | |
| | etc. | | | | | |

### Example 14: Method of Preparing Liquid Formulations

In embodiments, a formulation of sesquifumarate salt of Compound 1 at 30 mg/mL in pH 6.0 phosphate buffer is prepared for subcutaneous delivery to a subject as follows.

Initially, the buffer is prepared by weighing about 13.61 g of monobasic potassium phosphate (KH₂PO₄) into a 500 mL volumetric flask and make to volume with DI water—200 mM KH₂PO₄. Next, 50 mL of 200 mM KH₂PO₄ is added into a 200 mL volumetric flask and 5.6 mL 0.2 M NaOH is added. The pH is checked and adjusted if necessary, using either NaOH or HCl. Then volume is made with DI water—50 mM phosphate buffer (pH 6.0). Next, 20 mL of 50 mM phosphate buffer are added into a 100 mL volumetric flask and make to volume with DI water—10 mM phosphate buffer (pH 6.0).

Separately, 30 mg/mL of sesquifumarate salt of Compound 1 is prepared. About 39.9 mg of fumarate (1.5 eq. fumarate) salt of Compound 1 and 6.5 mg NaCl are weighed into a 1 mL volumetric flask. About ca. 0.9 mL of 10 mM phosphate buffer (pH 6.0) is added and vortex mixed. About 60 μL of 1M NaOH is added, and vortex mixed until fully dissolved. The pH is checked and adjusted, if necessary, with NaOH or HCl and volume is made with buffer. Finally, the 30 mg/ml of sesquifumarate salt of Compound 1 is added to the pH 6.0 10 mM phosphate buffer.

### Example 15: Succinate (1.5 Equivalents) Salt of Compound 1

Succinate (1.5 equivalents) salt of Compound 1 was prepared and analyzed. Analysis by XRPD shows the material to be crystalline (FIG. 13, Table 14).

TABLE 14
Diffraction Peaks for the Succinate
(1.5 equivalents) Salt of Compound 1

| Peak number | Peak Position °2θ | Relative Intensity |
|---|---|---|
| Peak 1 | 7.804 | 31.94% |
| Peak 2 | 8.701 | 62.43% |
| Peak 3 | 9.657 | 1.68% |
| Peak 4 | 10.778 | 12.36% |
| Peak 5 | 11.579 | 4.26% |
| Peak 6 | 11.988 | 4.50% |
| Peak 7 | 12.524 | 18.64% |
| Peak 8 | 13.887 | 1.78% |
| Peak 9 | 14.306 | 19.84% |
| Peak 10 | 14.451 | 35.90% |
| Peak 11 | 15.532 | 4.79% |
| Peak 12 | 15.853 | 1.86% |
| Peak 13 | 16.458 | 4.13% |
| Peak 14 | 16.742 | 26.61% |
| Peak 15 | 17.423 | 8.85% |
| Peak 16 | 17.812 | 4.39% |
| Peak 17 | 18.004 | 7.85% |
| Peak 18 | 18.543 | 55.84% |
| Peak 19 | 19.007 | 18.65% |
| Peak 20 | 19.712 | 100.00% |
| Peak 21 | 20.121 | 22.82% |
| Peak 22 | 21.209 | 7.32% |
| Peak 23 | 21.718 | 19.15% |
| Peak 24 | 22.038 | 29.03% |
| Peak 25 | 22.319 | 13.12% |
| Peak 26 | 23.083 | 44.13% |
| Peak 27 | 23.514 | 48.25% |
| Peak 28 | 23.918 | 15.83% |
| Peak 29 | 24.543 | 3.84% |
| Peak 30 | 24.889 | 4.35% |
| Peak 31 | 25.327 | 35.08% |
| Peak 32 | 26.876 | 4.41% |
| Peak 33 | 27.305 | 4.85% |
| Peak 34 | 28.108 | 2.68% |
| Peak 35 | 28.521 | 3.39% |
| Peak 36 | 28.858 | 1.16% |
| Peak 37 | 29.266 | 3.16% |
| Peak 38 | 30.117 | 2.58% |
| Peak 39 | 30.403 | 4.58% |
| Peak 40 | 30.745 | 2.14% |
| Peak 41 | 31.632 | 1.91% |
| Peak 42 | 33.166 | 3.65% |
| Peak 43 | 34.614 | 1.93% |
| Peak 44 | 34.898 | 3.33% |
| Peak 45 | 35.648 | 0.75% |
| Peak 46 | 36.586 | 2.77% |
| Peak 47 | 37.931 | 2.37% |
| Peak 48 | 38.573 | 1.88% |
| Peak 49 | 39.297 | 1.89% |

1. A liquid formulation comprising an effective amount of Compound 1 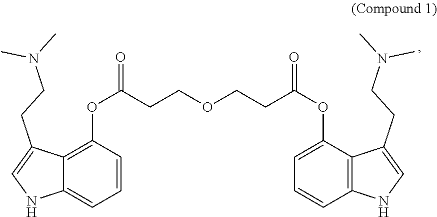
or an equivalent amount of a pharmaceutically acceptable salt thereof; and
a pharmaceutically acceptable excipient.

2. The liquid formulation of claim 1, wherein the salt is a fumarate salt of Compound 1, a succinate salt of Compound 1, or a tartrate salt of Compound 1.

3. The liquid formulation of claim 2, wherein the salt is a monofumarate salt of Compound 1.

4. The liquid formulation of claim 2, wherein the salt is a sesquifumarate salt of Compound 1.

5. The liquid formulation of claim 2, wherein the salt is a succinate salt of Compound 1.

6. The liquid formulation of claim 2, wherein the salt is a L-tartrate salt of Compound 1.

7. The liquid formulation of claim 2, wherein the salt is a phosphate salt of Compound 1.

8. The liquid formulation of claim 1, wherein the formulation comprises about 2 mg/mL to about 40 mg/mL of Compound 1, or an equivalent amount of a pharmaceutically acceptable salt thereof, relative to a total volume of the formulation.

9. The liquid formulation of claim 1, wherein the formulation comprises about 30 mg/mL to about 40 mg/mL of Compound 1, or an equivalent amount of a pharmaceutically acceptable salt thereof, relative to a total volume of the formulation.

10. The liquid formulation of claim 1, wherein the liquid formulation comprises a pH buffered aqueous solution.

11. The liquid formulation of claim 10, wherein the pH buffered aqueous solution comprises a phosphate buffer solution or a histidine buffer solution.

12. The liquid formulation of claim 11, wherein the pH buffered aqueous solution is a phosphate buffer solution having a phosphate concentration of about 1 mM to about 100 mM and a pH ranging from about 5.5 to about 6.5.

13. The liquid formulation of claim 11, wherein the pH buffered aqueous solution is a phosphate buffer solution having a phosphate concentration of about 10 mM and a pH of about 6.

14. The liquid formulation of claim 11, wherein the phosphate buffer solution is a phosphate-buffered saline (PBS).

15. The liquid formulation of claim 1, wherein the pharmaceutically acceptable excipient comprises a pH modifier.

16. The liquid formulation of claim 15, wherein the pH modifier is sodium hydroxide.

17. The liquid formulation of claim 1, wherein the formulation has a pH ranging from about 5.8 to about 8.5.

18. The liquid formulation of claim 17, wherein the formulation has a pH ranging from about 6 to about 7.5.

19. The liquid formulation of claim 18, wherein the formulation has a pH of 6.0±0.2, 6.5±0.2, or 7.4±0.2.

20. The liquid formulation of claim 1, wherein the formulation is acidic.

21. The liquid formulation of claim 1, wherein the formulation has a pH of 6.0±0.2.

22. The liquid formulation of claim 1, wherein the pharmaceutically acceptable excipient further comprises a tonicity agent.

23. The liquid formulation of claim 22, wherein the tonicity agent is sodium chloride or mannitol.

24. The liquid formulation of claim 23, wherein the tonicity agent is sodium chloride, and sodium chloride is present in the formulation in a concentration of about 0.1 wt % to about 5 wt %.

25. The liquid formulation of claim 1, wherein the formulation has an osmolarity ranging from 200 mOsm/kg to 600 mOsm/kg.

26. The liquid formulation of claim 25, wherein the formulation has an osmolarity ranging from about 280 mOsm/kg to about 400 mOsm/kg.

27. The liquid formulation of claim 26, wherein the formulation has an osmolarity of about 300 mOsm/kg.

28. The liquid formulation of claim 1, which is formulated for parenteral administration.

29. The liquid formulation of claim 28, which is formulated for subcutaneous administration.

30. The liquid formulation of claim 1, comprising:
about 30 mg/mL to about 40 mg/mL of Compound 1, or an equivalent amount of a pharmaceutically acceptable salt thereof, relative to a total volume of the formulation,
a phosphate buffer solution having a phosphate concentration of about 10 mM and a pH of about 6,
sodium hydroxide, and
about 0.1 wt % to about 5 wt % sodium chloride,
wherein the formulation is formulated for subcutaneous administration.

31. The liquid formulation of claim 1, comprising:
about 30 mg/mL to about 40 mg/ml of Compound 1 relative to a total volume of the formulation,
a phosphate buffer solution having a phosphate concentration of about 10 mM and a pH of about 6,
sodium hydroxide, and
about 0.1 wt % to about 5 wt % sodium chloride,
wherein the formulation is formulated for subcutaneous administration.

32. The liquid formulation of claim 1, comprising:
about 30 mg/mL to about 40 mg/ml of sesquifumarate salt of Compound 1 relative to a total volume of the formulation, wherein the concentration is determined based on the weight of Compound 1 as a free base,
a phosphate buffer solution having a phosphate concentration of about 10 mM and a pH of about 6,
sodium hydroxide, and
about 0.1 wt % to about 5 wt % sodium chloride,
wherein the formulation is formulated for subcutaneous administration.

33. The liquid formulation of claim 1, wherein the formulation has a shelf-life of at least 12 hours, 24 hours, or 48 hours at 25° C.

34. The liquid formulation of claim 1, wherein the formulation has a shelf-life of at least 4 days, 7 days, 14 days, 30 days, 2 months, or 3 months at 5° C.

35. A method of delivering Compound 1 or a pharmaceutically acceptable salt thereof to a subject, the method comprising administering the liquid formulation of claim 1 to the subject parenterally.

36. The method of claim 35, wherein the liquid formulation is administered to the subject subcutaneously.

37. The method of claim 35, wherein the liquid formulation is administered subcutaneously in a volume ranging from about 0.1 mL to about 2 mL.

38. The method of claim 35, wherein the subject is a human.

39. A method of treating a neuropsychiatric disease, the method comprising parenterally administering the liquid formulation of claim 1 to a subject in need thereof.

40. The method of claim 39, wherein the liquid formulation is administered to the subject subcutaneously.

41. The method of claim 39, wherein the liquid formulation is administered subcutaneously in a volume ranging from about 0.1 mL to about 2 mL.

42. The method of claim 39, wherein the subject is a human.

43. The method of claim 39, wherein the neuropsychiatric disorder is selected from anxiety disorder, attention deficit hyperactivity disorder (ADHD), depression (including treatment resistant depression), cluster headache, migraine, Parkinson's disease, schizophrenia, an eating disorder (including anorexia nervosa), psychotic disorder, schizophrenia, schizophreniform disorder, schizoaffective disorder, bipolar | disorder, bipolar II disorder, major depressive disorder, psychotic depression, delusional disorders, shared psychotic disorder, shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, attention deficit syndrome, posttraumatic stress disorder (PTSD), premenstrual dysphoric disorder (PMDD), and premenstrual syndrome (PMS).

44. A method of making a liquid formulation for parenteral administration to a subject, the method comprising:
combining an effective amount of a pharmaceutically acceptable salt of Compound 1; 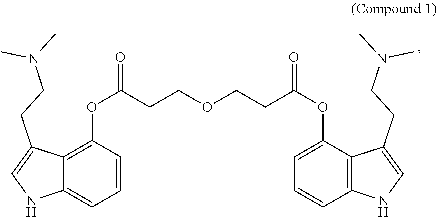
and
a pH buffered aqueous solution; and
optionally a pharmaceutically acceptable excipient thereby forming the liquid formulation.

45. The method of claim 44, wherein the method comprises combining an effective amount of the pharmaceutically acceptable salt of Compound 1, the pH buffered aqueous solution, and the pharmaceutically acceptable excipient.

46. The method of claim 44, wherein the salt of Compound 1 is crystalline.

47. The method of claim 46, wherein the salt is a sesquifumarate salt of Compound 1.

48. The method of claim 47, wherein the sesquifumarate salt of Compound 1 is crystalline, and is characterized by an XRPD pattern having peaks at 8.7±0.2, 19.7±0.2, and 23.6±0.2 °2θ.

49. The method of claim 48, wherein the sesquifumarate salt of Compound 1 is further characterized by peaks in an XRPD pattern at 14.4±0.2, 21.8±0.2, and 22.2±0.2 °2θ.

50. The method of claim 47, wherein the sesquifumarate salt of Compound 1 is characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °20.

51. The method of claim 44, wherein the pH buffered aqueous solution comprises a phosphate buffer solution or a histidine buffer solution.

52. The method of claim 51, wherein the pH buffered aqueous solution is a phosphate buffer solution having a phosphate concentration of about 1 mM to about 100 mM and a pH ranging from about 5.5 to about 6.5.

53. The method of claim 51, wherein the pH buffered aqueous solution is a phosphate buffer solution having a phosphate concentration of about 10 mM and a pH of about 6.

54. The method of claim 51, wherein the pH buffered aqueous solution is a phosphate buffer solution, and the phosphate buffer solution is a phosphate-buffered saline (PBS).

55. The method of claim 45, wherein the pharmaceutically acceptable excipient comprises a pH modifier.

56. The method of claim 55, wherein the pH modifier is sodium hydroxide.

57. The method of claim 45, wherein the pharmaceutically acceptable excipient further comprises a tonicity agent.

58. The method of claim 57, wherein the tonicity agent is sodium chloride or mannitol.

59. The method of claim 44, wherein the formulation has a pH ranging from about 5.8 to about 8.5.

60. The method of claim 59, wherein the formulation has a pH ranging from about 6 to about 7.5.

61. The method of claim 60, wherein the formulation has a pH of 6.0±0.2, 6.5±0.2, or 7.4±0.2.

62. The method of claim 44, wherein the formulation is acidic.

63. The method of claim 44, wherein the formulation has a pH of 6.0±0.2.

64. The method of claim 44, wherein the formulation has an osmolarity ranging from 200 mOsm/kg to 600 mOsm/kg.

65. The method of claim 64, wherein the formulation has an osmolarity ranging from about 280 mOsm/kg to about 400 mOsm/kg.

66. The method of claim 65, wherein the formulation has an osmolarity of about 300 mOsm/kg.

67. The method of claim 44, wherein the formulation is formulated for subcutaneous administration.

68. A first container, comprising about 1 mg to 100 mg of a pharmaceutically acceptable salt of Compound 1

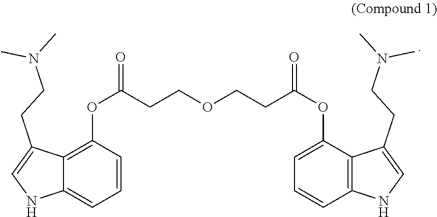

69. The first container of claim 68, wherein the salt of Compound 1 is in crystalline form.

70. The first container of claim 68, wherein the salt is a sesquifumarate salt of Compound 1.

71. The first container of claim 70, wherein the sesquifumarate salt of Compound 1 is crystalline, and is characterized by an XRPD pattern having peaks at 8.7±0.2, 19.7±0.2, and 23.6±0.2 °2θ.

72. The first container of claim 71, wherein the sesquifumarate salt of Compound 1 is further characterized by peaks in an XRPD pattern at 14.4±0.2, 21.8±0.2, and 22.2±0.2 °2θ.

73. The first container of claim 70, wherein the sesquifumarate salt of Compound 1 is characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °2θ.

74. A kit, comprising:
the first container of claim 68; and
a second container comprising a pH buffered aqueous solution.

75. The kit of claim 74, wherein the second container further comprises a pharmaceutically acceptable excipient.

76. The kit of claim 74, wherein the pH buffered aqueous solution is a phosphate buffer solution having a phosphate concentration of about 1 mM to about 100 mM and a pH ranging from about 5.5 to about 6.5.

77. The kit of claim 74, wherein the pH buffered aqueous solution is a phosphate-buffered saline (PBS).

78. The kit of claim 75, wherein the pharmaceutically acceptable excipient comprises a pH modifier.

79. The kit of claim 78, wherein the pH modifier is sodium hydroxide.

80. The kit of claim 78, wherein the pharmaceutically acceptable excipient further comprises a tonicity agent.

81. The kit of claim 80, wherein the tonicity agent is sodium chloride or mannitol.
